(19) United States
(12) Patent Application Publication
(10) Pub. No.: US 2023/0227567 A1
(43) Pub. Date: Jul. 20, 2023

(54) AGONIST ANTI-CD40 ANTIBODIES

(71) Applicant: Selvax Pty Ltd, West Perth (AU)

(72) Inventors: Delia Jane NELSON, West Perth (AU); Stephen PROKSCH, West Perth (AU); Jonny T. STINE, West Perth (AU)

(21) Appl. No.: 18/000,533

(22) PCT Filed: Jun. 4, 2021
(86) PCT No.: PCT/AU2021/050563

§ 371 Date: Dec. 2, 2022

(30) Foreign Application Priority Data
Jun. 4, 2020 (AU) 2020901845

(51) Int. Cl. C07K 16/28; A61K 45/06; C12N 15/63; A61K 38/20; A61P 35/00

(52) U.S. Cl. CPC C07K 16/2878; A61K 45/06; C12N 15/63; A61K 38/2013; A61P 35/00; A61K 2039/505

(57) ABSTRACT

The present application generally relates to the identification of certain agonist anti-CD40 antibodies. Based thereon, the invention provides novel agonist antibodies, and the use thereof in therapy.

### TECHNICAL FIELD

The present application generally relates to the identification of certain agonist anti-CD40 antibodies. Based thereon, the invention provides novel agonist antibodies, and the use thereof in therapy.

### BACKGROUND ART

Tumors are recognized by the host immune system at some point during their evolution. Despite evidence of recognition, some tumors thrive, possibly because they may lack danger signals and hence induce only weak responses, and possibly because under increasing immune pressure they develop immune evasion strategies.

Tumors treated in the early stages of their development are likely to demonstrate better responses to mono-immunotherapies. However, advanced cancer is more refractory to all forms of therapy, including immunotherapy. Their lack of danger signals plus their acquisition of increasingly complex immune escape mechanisms presents a significant handicap to the host adaptive immune response, which relies on antigen presenting cells (APCs) such as dendritic cells (DCs) to sample, process, and present tumor-derived antigen in the correct context with the appropriate co-stimulatory markers to provoke the relevant T-cell response. Tumor cells often do not present their own antigenic stimulation on account of their low expression levels of essential co-stimulatory molecules such as the B7 family members.

As tumor antigen cannot be directly presented, cross-presentation may be the only mode of natural antigen presentation for tumor immunity. During this process, exogenous antigen derived from the tumor cell (soluble antigen, apoptotic bodies, or live cancer cells) is taken up by DCs and, rather than just following the classical pathway of processing and presentation in the context of MHC class II molecules to elicit CD4+ T-cell help, the exogenous antigen is also internalized and displayed in the context of MHC class I molecules for presentation to CD8+ T cells. However, unless the presenting DCs are also appropriately activated and presentation occurs in the context of the appropriate co-stimulation, this process will result in weak responses or tolerance. DCs within the tumor environment are often in an immature state and can encourage the expansion of regulatory T cells in lymph nodes draining the tumor. Their function can also be suppressed by infiltrating suppressive cells such as myeloid-derived suppressor cells (MDSCs) and tumor-associated macrophages (TAMs) as well as by cytokines within the tumor and draining lymph node. Thus, effective immunotherapy that “helps” DCs to prime antigen-specific T-cell responses must overcome these roadblocks and aid the cross-presentation process. Effective cross-priming requires “licensed” DCs and high levels of antigen. “Licensing” or “conditioning” of DCs is carried out by antigen specific CD4+T helper cells, through cross-linking of CD40. This ligation alters DC phenotype and function, inhibiting their tolerogenic potential through autocrine signaling with cytokines such as IL-6 and IL-12, hence enabling them to activate potent cytotoxic T lymphocyte (CTL) responses. In contrast, CTLs activated by unlicensed DCs have been termed “helpless,” and T-cell anergy or deletion and regulatory T cells can also be induced as a result. The cell-surface molecule CD40, a member of the tumor necrosis factor receptor superfamily therefore broadly regulates immune activation and mediates tumor apoptosis.

CD40 is a transmembrane protein that is a member of the TNF receptor superfamily. CD40 is expressed by APCs and engagement of its natural ligand (CD154 or CD40L) on T helper cells and on platelets activates APCs including DCs, macrophages and B cells.

CD40 is found on a large portion of melanomas and carcinomas of the lung, breast, colon, prostate, pancreas, kidney, ovary, and head and neck as well as B cell malignancies.

Agonist anti-CD40 antibodies have been shown to substitute for T cell help provided by CD4+ lymphocytes in murine models of T cell-mediated immunity. In tumor-bearing hosts, CD40 agonists trigger effective immune responses against tumor-associated antigens. For example, DCs can be “preconditioned” using agonist anti-CD40 antibody such that they upregulate their co-stimulatory markers, enabling them to activate CD8+ T cells when they encounter them. Thus, agonist anti-CD40 antibody can substitute for CD4+ T-cell help by signaling CD40 on antigen-loaded DCs, causing upregulation of B7 co-stimulatory molecules and release of IL-12, thereby empowering them to stimulate a specific CTL response.

Thus, ligation of CD40 on the surface of APCs enhances the expression of MHC and costimulatory molecules such as CD86, CD80, CD83, PD-L1, HLA-A, B, C, or HLA-DR, stimulates the production of pro-inflammatory cytokines such as IL-10, IL-6, IL-10, IL-12p40, IL-12p70, IL-23 and IFN-γ and induces T cell activation, all of which are essential to cell-mediated immune responses.

Patients with germline mutations in either CD40 or CD40L are markedly immunosuppressed, susceptible to opportunistic infections, and have deficient T cell-dependent immune reactions including IgG production, germinal center formation, and memory B cell induction.

In murine models of T cell-mediated immunity, agonist CD40 antibodies have been shown to mimic the signal of CD40L and substitute for the function of CD4+ lymphocytes. Agonist CD40 antibodies can also overcome T cell tolerance in tumor-bearing mice, evoke effective cytotoxic T cell responses, and enhance the efficacy of anti-tumor vaccines.

These findings are in counter-distinction to ligation of CD40 on the surface of tumor cells, which in many cases mediates a direct cytotoxic effect resulting in tumor regression through apoptosis and necrosis. Although the exact function of CD40 on tumor cells is unclear, engagement of CD40 in vitro inhibits the growth of solid tumor cells and high-grade B cell lymphoma lines. In addition, CD40-mediated tumor inhibition has also been observed in vivo, including inhibition of breast carcinoma or B cell lymphoma xenografts in immunocompromised mice.

These diverse roles for CD40 provide an opportunity where activation of CD40 in a tumor-bearing animal has the potential of (i) a direct cytotoxic effect on the tumor, and (ii) provision of tumor antigens to APCs simultaneously activated by CD40.

Agonist monoclonal antibodies (mAb) to CD40 have already shown therapeutic activity in a range of preclinical models. These findings, along with the dual functionality of CD40, have made CD40 an attractive target for cancer therapy and form the rationale for the clinical development of agonist anti-CD40 antibodies.

There remains however a need for additional agonist monoclonal antibodies to human CD40. It is against this background that the present invention has been developed.

### SUMMARY OF INVENTION

The inventors have developed new agonist anti-CD40 antibodies suitable for use as an immunogenic agent capable of treating malignancies in a patient.

The agonist anti-CD40 antibodies of the invention are suitable for use in a variety of activities including without limitation, binding to: (a) CD40, tumor blood vessels such that they permit T cell traffic; (b) CD40, B cells in tumors, spleen, and lymph nodes such that they secrete increased levels of autoantibodies directed against antigen expressed on tumor cells; and (c) CD40, DCs and macrophages such that they upregulate costimulatory markers and release IL-12 to activate CD8, T cells and stimulate a specific CTL response to cross-presented tumor antigens. Further, when the antibodies of the invention are combined with other immune enhancing agents such as local or systemic IL-2, TLR-7 agonists, and/or cytotoxic chemotherapy, the generation of a potent antitumor CD8+ cytotoxic T lymphocyte response can be seen. Thus, the antibodies present and provide a new principal of general application in the field of neoplasm therapy.

In one form, the invention comprises an isolated agonist anti-CD40 antibody or fragment thereof which comprises (i) a V_{H} chain comprising three CDRs and (ii) a V_{L} chain comprising three CDRs, wherein one or more heavy chain complementary determining regions (CDRHs) is selected from the group consisting of:
a) a CDRH1 sequence comprising SEQ ID NO:1;
b) a CDRH2 sequence comprising SEQ ID NO:2;
c) a CDRH3 sequence comprising SEQ ID NO:3; or
d) any one of SEQ ID NO:1 to 3 that contains one or two amino acid substitutions, deletions or insertions.

In an embodiment of the first form of the invention, the invention comprises an isolated agonist anti-CD40 antibody or fragment thereof which comprises (i) a V_{H} chain comprising 3 CDRs and (ii) a V_{L} chain comprising three CDRs, wherein one or more heavy chain complementary determining regions (CDRHs) is selected from the group consisting of:
(a) a CDRH1 sequence comprising SEQ ID NO:1;
(b) a CDRH2 sequence comprising SEQ ID NO:2; or
(c) a CDRH2 sequence comprising SEQ ID NO:2 that contains one or two amino acid substitutions, deletions or insertions.

In an embodiment of the first form of the invention the isolated agonist anti-CD40 antibody comprises a heavy chain variable region of SEQ ID NO: 7.

In another embodiment of the first form of the invention, the isolated agonist anti-CD40 antibody additionally comprises one or more light chain complementary determining regions (CDRLs) selected from the group consisting of:
a) a CDRL1 sequence comprising SEQ ID NO:4;
b) a CDRL2 sequence comprising SEQ ID NO:5; or
c) a CDRL3 sequence comprising SEQ ID NO:6

In a second form, the invention comprises an isolated agonist anti-CD40 antibody or fragment thereof which comprises (i) a V_{H} chain comprising three CDRs and (ii) a V_{L} chain comprising three CDRs, wherein one or more CDRLs selected from the group consisting of:
a) a CDRL1 sequence comprising SEQ ID NO:4;
b) a CDRL2 sequence comprising SEQ ID NO:5; or
c) a CDRL3 sequence comprising SEQ ID NO:6
d) any one of SEQ ID NO:4 to 6 that contains one or two amino acid substitutions, deletions or insertions.

In an embodiment of the second form of the invention the isolated agonist anti-CD40 antibody comprises a light chain variable region of SEQ ID NO: 8.

In another embodiment of the second form of the invention, the isolated agonist anti-CD40 antibody additionally comprises one or more CDRHs selected from the group consisting of:
a) a CDRH1 sequence comprising SEQ ID NO:1;
b) a CDRH2 sequence comprising SEQ ID NO:2; or
c) a CDRH3 sequence comprising SEQ ID NO:3.

In a third form, the invention is an isolated agonist anti-CD40 antibody comprising:
a) a CDRH1 sequence comprising SEQ ID NO:1;
b) a CDRH2 sequence comprising SEQ ID NO:2;
c) a CDRH3 sequence comprising SEQ ID NO:3;
d) a CDRL1 sequence comprising SEQ ID NO:4;
e) a CDRL2 sequence comprising SEQ ID NO:5; and
f) a CDRL3 sequence comprising SEQ ID NO:6.

In an embodiment of the third form of the invention, the isolated agonist anti-CD40 antibody comprises a heavy chain variable region of SEQ ID NO: 7 and a light chain variable region of SEQ ID NO: 8.

In an embodiment of the above forms of the invention the agonist anti-CD40 antibodies can be a murine antibody, a monoclonal antibody, a polyclonal antibody, a recombinant antibody, a human antibody, a humanized antibody, a chimeric antibody, a multispecific antibody, or an antibody fragment thereof.

In an embodiment, the isolated agonist anti-CD40 antibody is a Fab fragment, a Fab′ fragment, a F(ab′)2 fragment, a Fv fragment, a diabody, or a single chain antibody molecule.

In an embodiment, the isolated agonist anti-CD40 antibody is a humanized antibody.

In an embodiment, the isolated agonist anti-CD40 antibody is a monoclonal antibody.

In an embodiment, the isolated agonist anti-CD40 antibody is of the IgG1-, IgG2a- IgG2b- IgG3- or IgG4-type. Preferably, the isolated agonist anti-CD40 antibody is of the IgG1 type.

In an embodiment, the isolated agonist anti-CD40 antibody is coupled to a labeling group.

In an embodiment, the isolated agonist anti-CD40 antibody enhances CD40 activity.

In an embodiment, the invention comprises a nucleic acid molecule encoding an isolated agonist anti-CD40 antibody as described herein.

In an embodiment, the invention comprises a vector comprising a nucleic acid molecule as described herein.

In an embodiment, the invention comprises a host cell comprising a nucleic acid molecule as described herein.

In an embodiment, the invention comprises an isolated agonist anti-CD40 antibody that is at least suitable for achieving at least one or more of the following functions:
a. promoting the secretion of autoantibodies directed against antigen expressed on tumour cells by B cells;
b. of upregulating costimulatory markers and releasing IL-12 to activate CD8+ T cells and stimulating a specific cytotoxic T-cell response to cross-presented tumour antigens;
c. increasing antigen presentation by APCs (including macrophages, DCs, and B cells);
d. enhancing the expression of MHC and immune costimulatory molecules (such as CD86, CD80, CD83, PD-L1, HLA-A, B, C, or HLA-DR);
e. stimulating the production of pro-inflammatory cytokines (such as IL-1(3, IL-6, IL-10, IL-12p40, IL-12p70, IL-23 and IFN-γ); or
f. inducing T cell activation;
g. mimicking the signal of CD40L and substituting for the function of CD4+ lymphocytes;
h. overcoming T cell tolerance in tumor-bearing subjects;
i. evoking effective cytotoxic T cell responses;
j. enhancing the efficacy of anti-tumor vaccines; or
k. rendering tumor vasculature more permissive to immune infiltrates.

In an embodiment, the invention is a pharmaceutical composition comprising at least one isolated agonist anti-CD40 antibody as described herein. Preferably the pharmaceutical composition includes a pharmaceutically acceptable excipient.

In an embodiment, the pharmaceutical composition can further comprise an active agent such as a radioisotope, radionuclide, a toxin, a therapeutic or a chemotherapeutic group.

In another form, the invention resides in a method of making an agonist anti-CD40 antibody as described herein, comprising the step of: preparing said agonist anti-CD40 antibody from a host cell that secretes said agonist anti-CD40 antibody.

In yet another form, the invention resides in a method for treating or preventing a condition associated with malignancy in a patient, comprising the step of: administering a therapeutically effective amount of at least one isolated agonist anti-CD40 antibody disclosed herein to a patient, in need of said antibody.

In an embodiment, the invention comprises a method for increasing antigen presentation by APCs (including macrophages, DCs, and B cells), in a subject comprising administering an effective amount of at least one isolated agonist anti-CD40 antibody disclosed herein.

In an embodiment, the invention comprises a method of activating antigen presenting cells in a subject comprising administering an effective amount of at least one isolated agonist anti-CD40 antibody disclosed herein.

In an embodiment, the invention comprises a method of enhancing the expression of MHC and/or immune costimulatory molecules in a subject comprising administering an effective amount of at least one isolated agonist anti-CD40 antibody disclosed herein. Preferably, the MHC and/or immune costimulatory molecules are selected from CD80, CD86, PD-L1, HLA-A, B, C, HLA-DR, and CD83.

In an embodiment, the invention comprises a method of stimulating the production of pro-inflammatory cytokines, in a subject comprising administering an effective amount of at least one agonist anti-CD40 antibody disclosed herein. Preferably the pro-inflammatory cytokine is selected from the list of IL-113, IL-6, IL-10, IL-12p40, IL-12p70, IL-23 and IFN-γ.

In an embodiment, the invention comprises a method of inducing T cell activation, in a subject comprising administering an effective amount of at least one agonist anti-CD40 antibody disclosed herein.

In an embodiment, the invention comprises a method to mimic the signal of CD40L and substitute for the function of CD4+ lymphocytes, in a subject comprising administering an effective amount of at least one agonist anti-CD40 antibody disclosed herein.

In an embodiment, the invention comprises a method for overcoming T cell tolerance in tumor-bearing animals or evoking effective cytotoxic T cell responses or enhance the efficacy of anti-tumor vaccines, in a subject comprising administering an effective amount of at least one agonist anti-CD40 antibody disclosed herein.

In an embodiment, the invention comprises a method of promoting the secretion of autoantibodies directed against antigen expressed on tumour cells by B cells, in a subject comprising administering an effective amount of an agonist anti-CD40 antibody disclosed herein.

In an embodiment, the invention comprises a method of upregulating costimulatory markers and releasing IL-12 to activate CD8+ T cells and stimulating a specific cytotoxic T-cell response to cross-presented tumour antigens in a subject comprising administering an effective amount of an agonist anti-CD40 antibody disclosed herein.

The complex orchestration of events required for tumor eradication suggests that a combined therapeutic approach can also be beneficial in some circumstances. Agonist anti-CD40 antibodies can be used with additional arms for releasing antigen, promoting cytokine release, increasing immunosurveillance, and reducing the suppressive network to boost this effect.

Accordingly, in one embodiment the invention comprises a pharmaceutical formulation comprising: an effective amount of at least one agonist anti-CD40 antibody disclosed herein together with one or more additional immune enhancing agents. Such immune enhancing agents include, without limitation, IL-2, TLR-7 agonists, or systemic cytotoxic chemotherapeutic agents.

In another embodiment, the invention comprises a method for treating or preventing a condition associated with malignancy in a patient, comprising administering to a patient in need thereof an effective amount of at least one isolated agonist anti-CD40 antibody disclosed herein and an effective amount of at least a second immune enhancing agent.

One therapeutic option is to alter the tumor microenvironment itself, encouraging the tumor to act as its own source of antigenic stimulation. This can be achieved by introducing IL-2 with anti-CD40 antibody into the tumor site. When directly co-injected the co-administration can avoid the toxicity associated with systemic administration and successfully cause regression of larger tumors, as well as distal tumors, while preserving long-term protective memory. Co-administration of IL-2 and a CD40 agonist can lead to increased macrophage activity and B cell activation. A combination of IL-2 and a CD40 agonist can therefore display remarkable benefit against various cancers with regression linked to a neutrophil dominant inflammatory response.

Accordingly, in an embodiment, the invention comprises a pharmaceutical composition comprising at least one agonist anti-CD40 antibody described herein and IL-2.

In an embodiment, the invention comprises a method for treating or preventing a condition associated with malignancy in a patient, comprising administering to a patient in need thereof an effective amount of each of, at least one agonist anti-CD40 antibody described herein and IL-2.

In an embodiment, the invention comprises a method for increasing antigen presentation by APCs (including macrophages, DCs, and B cells), in a subject comprising administering an effective amount of each of, at least one agonist anti-CD40 antibody described herein and IL-2.

In an embodiment, the invention comprises a method of activating antigen presenting cells in a subject comprising administering an effective amount of at least one isolated agonist anti-CD40 antibody disclosed herein and IL-2.

In an embodiment, the invention comprises a method of enhancing the expression of MHC and/or immune costimulatory molecules in a subject comprising administering an effective amount of each of, at least one agonist anti-CD40 antibody described herein and IL-2. Preferably, the MHC and/or immune costimulatory molecules are selected from CD80, CD86, PD-L1, HLA-A, B, C, HLA-DR, and CD83.

In an embodiment, the invention comprises a method of stimulating the production of pro-inflammatory cytokines, in a subject comprising administering an effective amount of each of, at least one agonist anti-CD40 antibody described herein and IL-2. Preferably the pro-inflammatory cytokine is selected from a list of IL-113, IL-6, IL-10, IL-12p40, IL-12p70, IL-23 and IFN-γ.

In an embodiment, the invention comprises a method of inducing T cell activation, in a subject comprising administering an effective amount of each of, at least one agonist anti-CD40 antibody described herein and IL-2.

In an embodiment, the invention comprises a method to mimic the signal of CD40L and substitute for the function of CD4+ lymphocytes, in a subject comprising administering an effective amount of each of, at least one agonist anti-CD40 antibody described herein and IL-2.

In an embodiment, the invention comprises a method for overcoming T cell tolerance in tumor-bearing animals or evoking effective cytotoxic T cell responses or enhancing the efficacy of anti-tumor vaccines, in a subject comprising administering an effective amount of each of, at least one agonist anti-CD40 antibody described herein and IL-2.

In an embodiment, the invention comprises a method of promoting the secretion of autoantibodies directed against antigen expressed on tumour cells by B cells, in a subject comprising administering an effective amount of an agonist anti-CD40 antibody disclosed herein and IL-2.

In an embodiment, the invention comprises a method of upregulating costimulatory markers and releasing IL-12 to activate CD8+ T cells and stimulating a specific cytotoxic T-cell response to cross-presented tumour antigens in a subject comprising administering an effective amount of an agonist anti-CD40 antibody disclosed herein and IL-2.

The most effective method for treating or preventing a condition associated with malignancy in a patient may require a combined therapeutic approach in which therapeutic interventions are carried out in sequence over time.

Accordingly, in an embodiment the invention comprises a method for treating or preventing a condition associated with malignancy in a patient, comprising administering to a patient in need thereof an effective amount of at least one isolated agonist anti-CD40 antibody disclosed herein in sequence over time with an additional therapeutic intervention. In some embodiments, said additional therapeutic intervention is selected from the group consisting of surgery, radiotherapy, chemotherapy, thermotherapy and immunotherapy.

One therapeutic option is to alter cells isolated from a patient and then transfer these cells back into the patient. This can be achieved by treating cells isolated from a patient with anti CD40 antibody. Cells treated with anti CD40 antibody may be treated with additional agents. In some embodiments, said agents are selected from the group consisting of tumor-specific peptides, tumor cell lysates, cytokines, agonists and mitogens.

Accordingly, in an embodiment the invention comprises a method for treating or preventing a condition associated with malignancy in a patient, comprising administering to a patient in need thereof cells treated with an effective amount of at least one isolated agonist anti-CD40 antibody disclosed herein. In some embodiments, said cells have been isolated from the patient and are selected from the group consisting of DCs, macrophages, B cells, myeloid cells, lymphoid cells and haematopoietic stem cells.

Additional objectives, advantages and novel features will be set forth in the description which follows or will become apparent to those skilled in the art upon examination of the drawings and the ensuing detailed description of several non-limiting embodiments which follows.

### BRIEF DESCRIPTION OF THE DRAWINGS

The disclosure will provide details in the following description of preferred embodiments with reference to the following figures wherein:

FIG. 1 presents the nucleotide sequence for the heavy chain of a humanised agonist anti-human CD40 antibody.

FIG. 2 presents the nucleotide sequence nucleotide sequence for the light chain of a humanised agonist anti-human CD40 antibody.

FIG. 3 presents the nucleotide and amino acid sequences of the heavy chain variable region (VH) of antibody SVX-3001.

FIG. 4 presents the nucleotide and amino acid sequences of the light chain variable region (VL) of antibody SVX-3001.

FIG. 5 presents the nucleotide and amino acid sequences of the synthesised gene sequence encoding the heavy chain of antibody SVX-3001 that was cloned into plasmid pcDNA3.1(+) to produce plasmid pcDNA3.1(+)_Selvax01HC.

FIG. 6 present the nucleotide and amino acid sequences of the synthesised gene sequence encoding the light chain of antibody SVX-3001 that was cloned into plasmid pcDNA3.1(+) to produce plasmid pcDNA3.1(+)_Selvax01LC.

FIG. 7 shows the plasmid map of pcDNA3.1(+)_Selvax01HC

FIG. 8 shows the plasmid map of pcDNA3.1(+) Selvax01LC

FIG. 9 shows the results of an ELISA assay showing the produced antibody was a CD40-specific IgG.

FIG. 10 shows the results of a FACS analysis detecting the binding of SVX-3001 on cells expressing CD40.

FIG. 11 shows the results of a CFSE assay to detect cell division in human PBMC in response to stimulation with SVX-3001.

FIG. 12 presents the results of a LEGENDplex assay to detect cytokine production from human PBMC in response to stimulation with SVX-3001 with and without IL-2.

FIG. 13 presents the results of a FACS assay determining the ability of SVX-3001 to block the binding of antibodies B-B20 and LOB7/6 to CD40.

FIG. 14 presents Biacore T200 sensograms for epitope mapping of SVX-3001 in relation to a number of different CD40 antibodies.

FIG. 15 presents the results of a FACS assay determining the activation of monocyte derived dendritic cells (moDC) with SVX-3001.

FIG. 16 presents the results of a FACS assay determining the dose response of monocyte derived dendritic cells (moDC) to SVX-3001

FIG. 17 presents the nucleotide sequence of the synthesised gene sequence encoding the heavy chain of antibody SVX-3001 that was cloned into plasmid pcDNA3.4-TOPO to produce plasmid 20ACGJQC_Selvax01HC-pcDNA3.4-TOPO.

FIG. 18 present the nucleotide and amino acid sequences of the synthesised gene sequence encoding the light chain of antibody SVX-3001 that was cloned into plasmid pcDNA3.4-TOPO to produce plasmid 20ACGJRC_Selvax01LC-pcDNA3.4-TOPO.

FIG. 19 shows the plasmid map of 20ACGJQC_Selvax01HC-pcDNA3.4-TOPO.

FIG. 20 shows the plasmid map of 20ACGJRC_Selvax01LC-pcDNA3.4-TOPO.

### BRIEF DESCRIPTION OF THE SEQUENCE LISTINGS

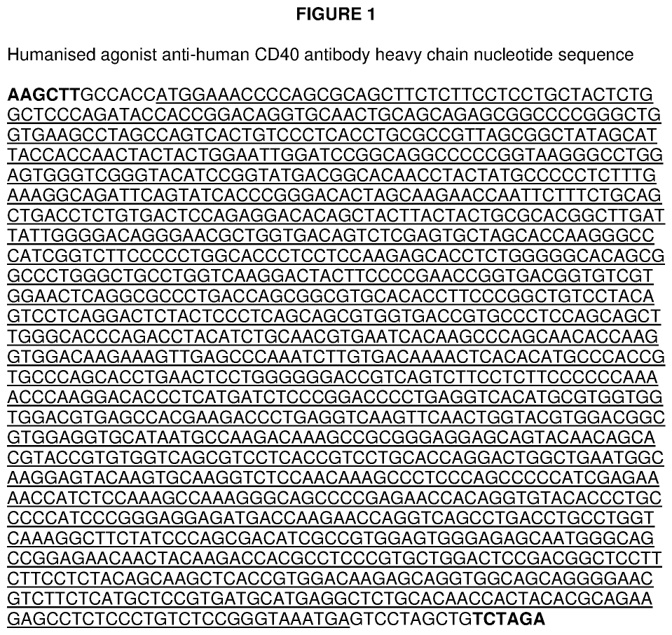

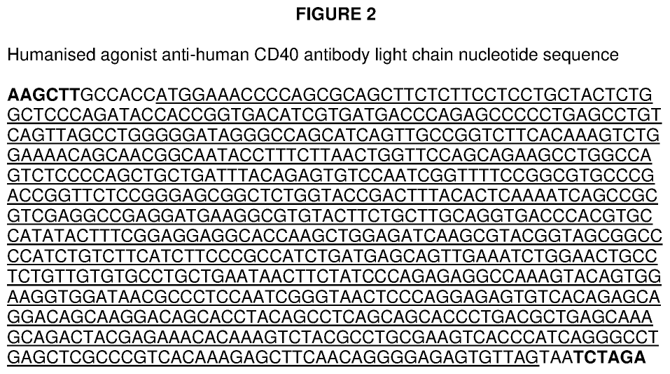

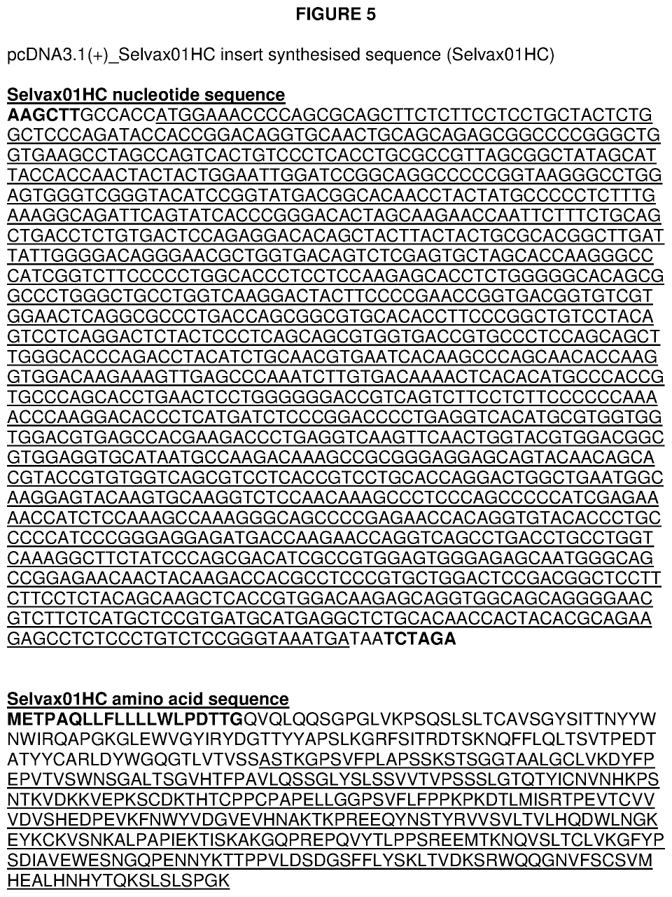

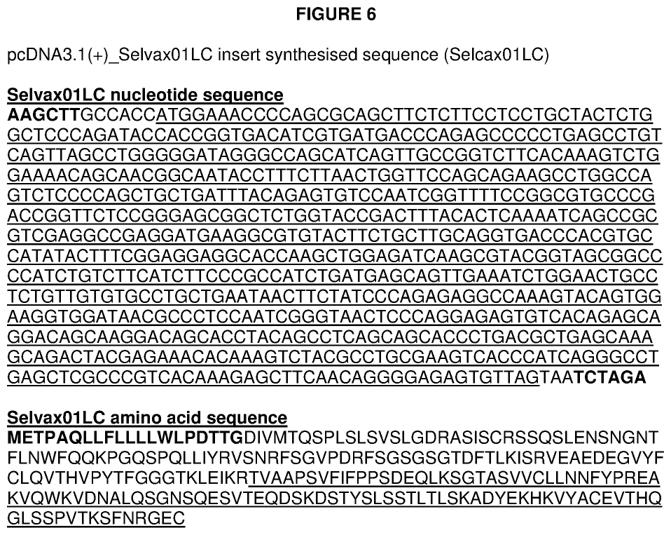

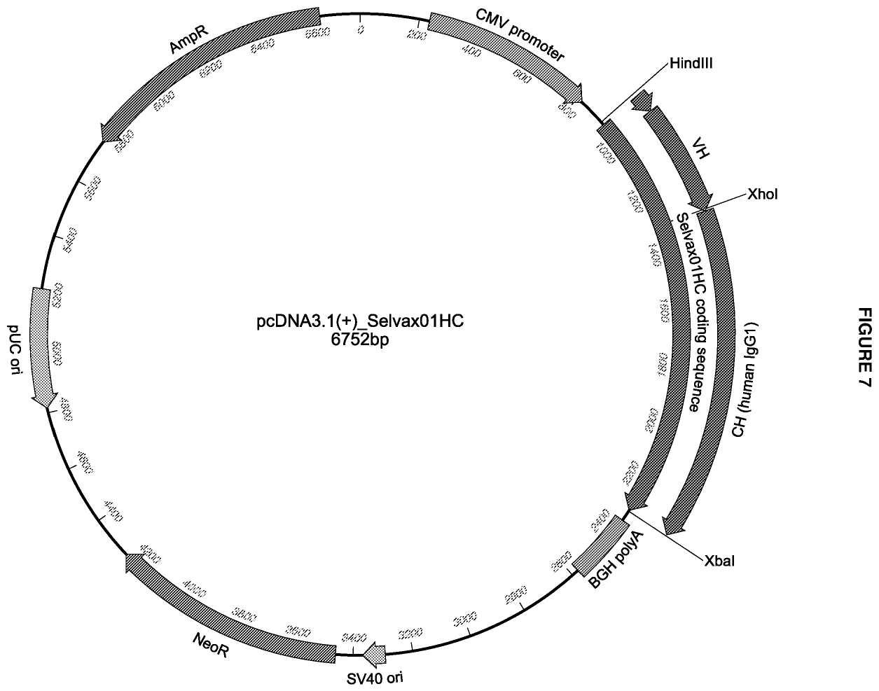

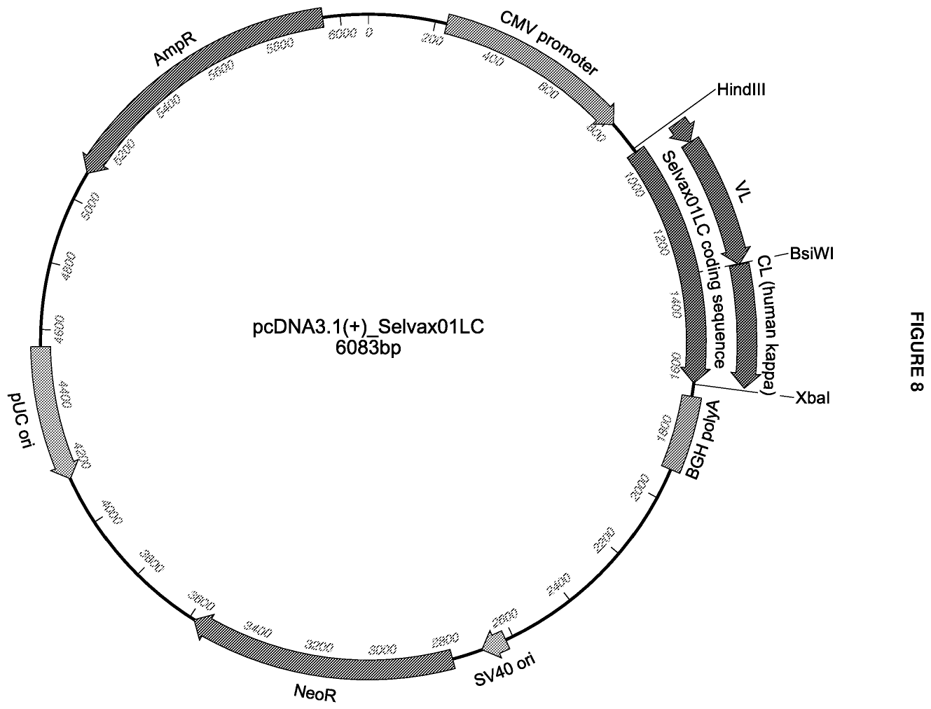

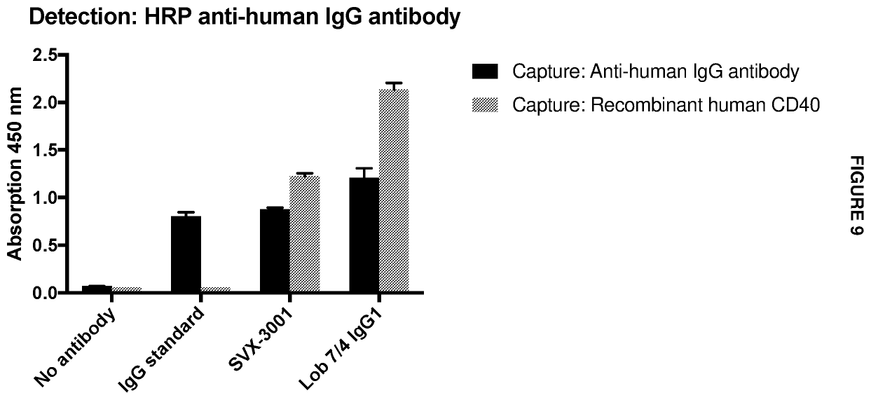

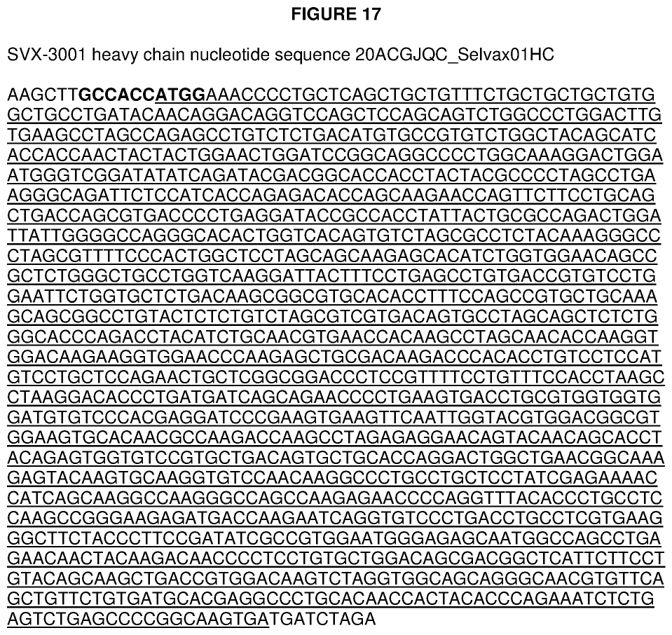

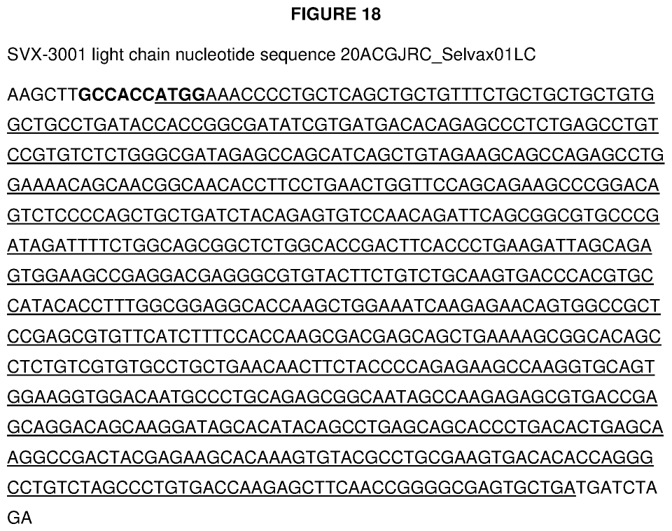

TABLE 1
List of Sequences

| SEQ ID NO | Descriptor | SEQUENCE |
|---|---|---|
| SEQ ID NO 1 | Humanized anti-human CD- | GYSITTNYYWN |
| | 40 antibody SVX-3001 | |
| | Heavy-chain variable region | |
| | (VH) CDR1 | |
| SEQ ID NO 2 | Humanized anti-human CD- | YIRYDGTTYYAPSLKG |
| | 40 antibody SVX-3001 | |
| | Heavy-chain variable region | |
| | (VH) CDR2 | |
| SEQ ID NO 3 | Humanized anti-human CD- | LDY |
| | 40 antibody SVX-3001 | |
| | Heavy-chain variable region | |
| | (VH) CDR3 | |
| SEQ ID NO 4 | Humanized anti-human CD- | RSSQSLENSNGNTFLN |
| | 40 antibody SVX-3001 | |
| | Light-chain variable region | |
| | (VL) CDR1 | |
| SEQ ID NO 5 | Humanized anti-human CD- | RVSNRFS |
| | 40 antibody SVX-3001 | |
| | Light-chain variable region | |
| | (VL) CDR2 | |
| SEQ ID NO 6 | Humanized anti-human CD- | LQVTHVPYT |
| | 40 antibody SVX-3001 | |
| | Light-chain variable region | |
| | (VL) CDR3 | |
| SEQ ID NO 7 | Humanized anti-human CD- | QVQLQQSGPGLVKPSQSLSLTCAVS |
| | 40 antibody SVX-3001 | GYSITTNYYWNWIRQAPGKGLEWVG |
| | Heavy-chain variable region | YIRYDGTTYYAPSLKGRFSITRDTSKN |
| | (VH) | QFFLQLTSVTPEDTATYYCARLDYWG |
| | | QGTLVTVSS |
| SEQ ID NO 8 | Humanized anti-human CD- | DIVMTQSPLSLSVSLGDRASISCRSSQ |
| | 40 antibody SVX-3001 | SLENSNGNTFLNWFQQKPGQSPQLLI |
| | Light-chain variable region | YRVSNRFSGVPDRFSGSGSGTDFTL |
| | (VL) | KISRVEAEDEGVYFCLQVTHVPYTFG |
| | | GGTKLEIKR |
| SEQ ID NO 9 | Humanized anti-human CD- | CAGGTGCAACTGCAGCAGAGCGGC |
| | 40 antibody SVX-3001 | CCCGGGCTGGTGAAGCCTAGCCAG |
| | Heavy-chain variable region | TCACTGTCCCTCACCTGCGCCGTTA |
| | (VH) Nucleotide Sequence | GCGGCTATAGCATTACCACCAACTAC |
| | | TACTGGAATTGGATCCGGCAGGCCC |
| | | CCGGTAAGGGCCTGGAGTGGGTCG |
| | | GGTACATCCGGTATGACGGCACAAC |
| | | CTACTATGCCCCCTCTTTGAAAGGCA |
| | | GATTCAGTATCACCCGGGACACTAG |
| | | CAAGAACCAATTCTTTCTGCAGCTGA |
| | | CCTCTGTGACTCCAGAGGACACAGC |
| | | TACTTACTACTGCGCACGGCTTGATT |
| | | ATTGGGGACAGGGAACGCTGGTGA |
| | | CAGTCTCGAGT |
| SEQ ID NO 10 | Humanized anti-human CD- | GACATCGTGATGACCCAGAGCCCCC |
| | 40 antibody SVX-3001 | TGAGCCTGTCAGTTAGCCTGGGGGA |
| | Light-chain variable region | TAGGGCCAGCATCAGTTGCCGGTCT |
| | (VL) Nucleotide Sequence | TCACAAAGTCTGGAAAACAGCAACG |
| | | GCAATACCTTTCTTAACTGGTTCCAG |
| | | CAGAAGCCTGGCCAGTCTCCCCAG |
| | | CTGCTGATTTACAGAGTGTCCAATCG |
| | | GTTTTCCGGCGTGCCCGACCGGTTC |
| | | TCCGGGAGCGGCTCTGGTACCGAC |
| | | TTTACACTCAAAATCAGCCGCGTCG |
| | | AGGCCGAGGATGAAGGCGTGTACTT |
| | | CTGCTTGCAGGTGACCCACGTGCCA |
| | | TATACTTTCGGAGGAGGCACCAAGC |
| | | TGGAGATCAAGCGT |
| SEQ ID NO 11 | Human full length CD40 | MVRLPLQCVLWGCLLTAVHPEPPTAC |
| | | REKQYLINSQCCSLCQPGQKLVSDCT |
| | | EFTETECLPCGESEFLDTWNRETHCH |
| | | QHKYCDPNLGLRVQQKGTSETDTICT |
| | | CEEGWHCTSEACESCVLHRSCSPGF |
| | | GVKQIATGVSDTICEPCPVGFFSNVSS |
| | | AFEKCHPWTSCETKDLVVQQAGTNK |
| | | TDVVCGPQDRLRALVVIPIIFGILFAILL |
| | | VLVFIKKVAKKPTNKAPHPKQEPQEIN |
| | | FPDDLPGSNTAAPVQETLHGCQPVTQ |
| | | EDGKESRISVQERQ |
| SEQ ID NO 12 | polyhistidine tag | HHHHHH |
| SEQ ID NO 13 | Humanized anti-human CD- | See FIG. 1 |
| | 40 antibody SVX-3001 | |
| | Heavy-chainNucleotide | |
| | Sequence | |
| SEQ ID NO 14 | Humanized anti-human CD- | See FIG. 2 |
| | 40 antibody SVX-3001 | |
| | Light-chainNucleotide | |
| | Sequence | |
| SEQ ID NO 15 | Selvax01HC nucleotide | See FIG. 5 |
| | sequence | |
| SEQ ID NO 16 | Selvax01HC amino acid | See FIG. 5 |
| | sequence | |
| SEQ ID NO 17 | Selvax01LC nucleotide | See FIG. 6 |
| | sequence | |
| SEQ ID NO 18 | Selvax01LC amino acid | See FIG. 6 |
| | sequence | |
| SEQ ID NO 19 | Humanized anti-human CD- | QVQLQQSGPGLVKPSQSLSLTCAVS |
| | 40 antibody SVX-3001 | GYSITTNYYWNWIRQAPGKGLEWVG |
| | Heavy Chain Amino Acid | YIRYDGTTYYAPSLKGRFSITRDTSKN |
| | Sequence | QFFLQLTSVTPEDTATYYCARLDYWG |
| | | QGTLVTVSSASTKGPSVFPLAPSSKS |
| | | TSGGTAALGCLVKDYFPEPVTVSWNS |
| | | GALTSGVHTFPAVLQSSGLYSLSSVV |
| | | TVPSSSLGTQTYICNVNHKPSNTKVD |
| | | KKVEPKSCDKTHTCPPCPAPELLGGP |
| | | SVFLFPPKPKDTLMISRTPEVTCVVVD |
| | | VSHEDPEVKFNWYVDGVEVHNAKTK |
| | | PREEQYNSTYRVVSVLTVLHQDWLN |
| | | GKEYKCKVSNKALPAPIEKTISKAKGQ |
| | | PREPQVYTLPPSREEMTKNQVSLTCL |
| | | VKGFYPSDIAVEWESNGQPENNYKTT |
| | | PPVLDSDGSFFLYSKLTVDKSRWQQ |
| | | GNVFSCSVMHEALHNHYTQKSLSLSP |
| | | GK |
| SEQ ID NO 20 | Humanized anti-human CD- | DIVMTQSPLSLSVSLGDRASISCRSSQ |
| | 40 antibody SVX-3001 | SLENSNGNTFLNWFQQKPGQSPQLLI |
| | Light-chain Amino Acid | YRVSNRFSGVPDRFSGSGSGTDFTL |
| | Sequence | KISRVEAEDEGVYFCLQVTHVPYTFG |
| | | GGTKLEIKRTVAAPSVFIFPPSDEQLK |
| | | SGTASVVCLLNNFYPREAKVQWKVD |
| | | NALQSGNSQESVTEQDSKDSTYSLSS |
| | | TLTLSKADYEKHKVYACEVTHQGLSS |
| | | PVTKSFNRGEC |
| SEQ ID NO 21 | 20ACGJQC_Selvax01HC | See FIG. 17 |
| | nucleotide sequence | |
| SEQ ID NO 22 | 20ACGJRC_Selvax01LC | See FIG. 18 |
| | nucleotide sequence | |

### DETAILED DESCRIPTION OF THE INVENTION

The present invention is directed to isolated agonist anti-human CD40 antibodies that are capable of, inter alia: increasing antigen presentation by APCs (including macrophages, DCs, and B cells). In certain embodiments the antibodies provide a method of enhancing the expression of MHC and/or immune costimulatory molecules. They can also stimulate the production of pro-inflammatory cytokines, inducing T cell activation. They do this by mimicking the signal of CD40L and substituting for the function of CD4+ lymphocytes. In doing this, isolated agonist anti-CD40 antibodies described here are capable of ameliorating T cell tolerance in tumor-bearing animals, can evoke effective cytotoxic T cell responses and/or enhance the efficacy of anti-tumor vaccines.

For convenience, the following sections generally outline the various meanings of the terms used herein. Following this discussion, general aspects regarding agonist anti-CD40 antibodies are discussed, followed by specific examples demonstrating the properties of various embodiments of the antibodies and how they can be employed.

### Definitions

The present invention is not to be limited in scope by the following specific embodiments. This detailed description is intended for the purpose of exemplification only. Functionally equivalent products, compositions and methods are within the scope of the invention as described herein. Consistent with this position, those skilled in the art will appreciate that the invention described herein is susceptible to variations and modifications other than those specifically described. It is to be understood that the invention includes all such variations and modifications. The invention also includes all of the steps, features, compositions and compounds referred to or indicated in the specification, individually or collectively and any and all combinations or any two or more of the steps or features.

In this application, the use of the singular includes the plural unless specifically stated otherwise. In this application, the use of “or” means “and/or” unless stated otherwise. Furthermore, the use of the term “including”, as well as other forms, such as “includes” and “included”, is not limiting. Also, terms such as “element” or “component” encompass both elements and components comprising one unit and elements and components that comprise more than one subunit unless specifically stated otherwise. Also, the use of the term “portion” can include part of a moiety or the entire moiety.

The term “antibody” refers to an intact immunoglobulin of any isotype, or a fragment thereof that can compete with the intact antibody for specific binding to the target antigen, and includes, for instance, chimeric and bispecific antibodies. An intact antibody will generally comprise at least two full-length heavy chains and two full-length light chains, but in some instances can include fewer chains such as antibodies naturally occurring in camelids which can comprise only heavy chains. Antibodies can be derived solely from a single source, or can be “chimeric,” that is, different portions of the antibody can be derived from two different antibodies as described further below. The antibodies, or binding fragments can be produced in hybridomas, by recombinant DNA techniques, or by enzymatic or chemical cleavage of intact antibodies. Unless otherwise indicated, the term “antibody” includes, in addition to antibodies comprising two full-length heavy chains and two full-length light chains, derivatives, variants, fragments, and muteins thereof, examples of which are described below. Furthermore, unless explicitly excluded, antibodies include monoclonal antibodies, bispecific antibodies, minibodies, domain antibodies, synthetic antibodies (sometimes referred to herein as “antibody mimetics”), chimeric antibodies, antibody fusions (sometimes referred to herein as “antibody conjugates”), and fragments thereof, respectively. In some embodiments, the term also encompasses peptibodies.

Antibody heavy chains are typically classified as mu, delta, gamma, alpha, or epsilon, and define the antibody's isotype as IgM, IgD, IgG, IgA, and IgE, respectively. IgG has several subclasses, including, but not limited to, IgG1, IgG2, IgG3, and IgG4. IgM has subclasses including, but not limited to, IgM1 and IgM2. IgA is similarly subdivided into subclasses including, but not limited to, IgA1 and IgA2. Within full-length light and heavy chains, typically, the variable and constant regions are joined by a “J” region of about 12 or more amino acids, with the heavy chain also including a “D” region of about 10 more amino acids. See, e.g., Fundamental Immunology, Ch. 7 (Paul, W., ed., 2nd ed. Raven Press, N.Y. (1989)) (incorporated by reference in its entirety for all purposes). The variable regions of each light/heavy chain pair typically form the antigen binding site.

The variable regions typically exhibit the same general structure of relatively conserved framework regions (FR) joined by three hyper variable regions, also called complementarity determining regions or CDRs. The CDRs from the two chains of each pair typically are aligned by the framework regions, which can enable binding to a specific epitope. From N-terminal to C-terminal, both light and heavy chain variable regions typically comprise the domains FR1, CDR1, FR2, CDR2, FR3, CDR3 and FR4. The assignment of amino acids to each domain is typically in accordance with the definitions of Kabat Sequences of Proteins of Immunological Interest (National Institutes of Health, Bethesda, Md. (1987 and 1991)), or Chothia & Lesk, J. Mol. Biol., 196:901-917 (1987); Chothia et al., Nature, 342:878-883 (1989).

In certain embodiments, an antibody heavy chain binds to an epitope in the absence of an antibody light chain. In certain embodiments, an antibody light chain binds to an epitope in the absence of an antibody heavy chain. In certain embodiments, an antibody binding region binds to an epitope in the absence of an antibody light chain. In certain embodiments, an antibody binding region binds to an epitope in the absence of an antibody heavy chain. In certain embodiments, an individual variable region specifically binds to an epitope in the absence of other variable regions.

In certain embodiments, definitive delineation of a CDR and identification of residues comprising the binding site of an antibody is accomplished by solving the structure of the antibody and/or solving the structure of the antibody-ligand complex. In certain embodiments, that can be accomplished by any of a variety of techniques known to those skilled in the art, such as X-ray crystallography. In certain embodiments, various methods of analysis can be employed to identify or approximate the CDR regions. Examples of such methods include, but are not limited to, the Kabat definition, the Chothia definition, the AbM definition and the contact definition.

By convention, the CDR regions in the heavy chain are typically referred to as H1, H2, and H3 and are numbered sequentially in the direction from the amino terminus to the carboxy terminus. The CDR regions in the light chain are typically referred to as L1, L2, and L3 and are numbered sequentially in the direction from the amino terminus to the carboxy terminus.

The term “light chain” includes a full-length light chain and fragments thereof having sufficient variable region sequence to confer binding specificity. A full-length light chain includes a variable region domain, V_{L}, and a constant region domain, C_{L}. The variable region domain of the light chain is at the amino-terminus of the polypeptide. Light chains include kappa chains and lambda chains.

The term “heavy chain” includes a full-length heavy chain and fragments thereof having sufficient variable region sequence to confer binding specificity. A full-length heavy chain includes a variable region domain, V_{H}, and three constant region domains, C_{H}1, C_{H}2, and C_{H}3. The V_{H} domain is at the amino-terminus of the polypeptide, and the C_{H} domains are at the carboxyl-terminus, with the C_{H}3 being closest to the carboxy-terminus of the polypeptide. Heavy chains can be of any isotype, including IgG (including IgG1, IgG2, IgG3 and IgG4 subtypes), IgA (including IgA1 and IgA2 subtypes), IgM and IgE.

A bispecific or bifunctional antibody typically is an artificial hybrid antibody having two different heavy/light chain pairs and two different binding sites. Bispecific antibodies can be produced by a variety of methods including, but not limited to, fusion of hybridomas or linking of Fab′ fragments. See, e.g., Songsivilai et al., Clin. Exp. Immunol., 79: 315-321 (1990); Kostelny et al., J. Immunol., 148:1547-1553 (1992).

Each individual immunoglobulin chain is typically composed of several “immunoglobulin domains,” each consisting of roughly 90 to 110 amino acids and having a characteristic folding pattern. These domains are the basic units of which antibody polypeptides are composed. In humans, the IgA and IgD isotypes contain four heavy chains and four light chains; the IgG and IgE isotypes contain two heavy chains and two light chains; and the IgM isotype contains five heavy chains and five light chains. The heavy chain C region typically comprises one or more domains that can be responsible for effector function. The number of heavy chain constant region domains will depend on the isotype. IgG heavy chains, for example, contain three C region domains known as C_{H}1, C_{H}2 and C_{H}3. The antibodies that are provided can have any of these isotypes and subtypes.

The term “variable region” or “variable domain” refers to a portion of the light and/or heavy chains of an antibody, typically including approximately the amino-terminal 120 to 130 amino acids in the heavy chain and about 100 to 110 amino terminal amino acids in the light chain. In certain embodiments, variable regions of different antibodies differ extensively in amino acid sequence even among antibodies of the same species. The variable region of an antibody typically determines specificity of a particular antibody for its target.

The term “epitope” includes any determinant(s) capable of being bound by an antibody or to a T-cell receptor. An epitope is a region that engages with an antibody or with a T-cell receptor that targets that epitope, and when the epitope is part of a protein, includes specific amino acids that directly contact the antibody or to a T-cell receptor. Most often, epitopes reside on proteins, but in some instances can reside on other kinds of molecules, such as nucleic acids. Epitope determinants can include chemically active surface groupings of molecules such as amino acids, sugar side chains, phosphoryl or sulfonyl groups, and can have specific three-dimensional structural characteristics, and/or specific charge characteristics. Generally, antibodies specific for a particular target epitope will preferentially recognize an epitope on the target in a complex mixture of proteins and/or macromolecules.

The term “polynucleotide” or “nucleic acid” includes both single-stranded and double-stranded nucleotide polymers. The nucleotides comprising the polynucleotide can be ribonucleotides or deoxyribonucleotides or a modified form of either type of nucleotide. Said modifications include base modifications such as bromouridine and inosine derivatives, ribose modifications such as 2′,3′-dideoxyribose, and internucleotide linkage modifications such as phosphorothioate, phosphorodithioate, phosphoroselenoate, phosphorodiselenoate, phosphoroanilothioate, phoshoraniladate and phosphoroamidate.

The term “oligonucleotide” means a polynucleotide comprising 200 or fewer nucleotides. In some embodiments, oligonucleotides are 10 to 60 bases in length. In other embodiments, oligonucleotides are 12, 13, 14, 15, 16, 17, 18, 19, or 20 to 40 nucleotides in length. Oligonucleotides can be single stranded or double stranded, e.g., for use in the construction of a mutant gene. Oligonucleotides can be sense or antisense oligonucleotides. An oligonucleotide can include a label, including a radiolabel, a fluorescent label, a hapten or an antigenic label, for detection assays. Oligonucleotides can be used, for example, as PCR primers, cloning primers or hybridization probes.

An “isolated nucleic acid molecule” means a DNA or RNA of genomic, mRNA, cDNA, or synthetic origin or some combination thereof which is not associated with all or a portion of a polynucleotide in which the isolated polynucleotide is found in nature, or is linked to a polynucleotide to which it is not linked in nature. Isolated nucleic acid molecules “comprising” specified nucleic acid sequences can include, in addition to the specified sequences, coding sequences for up to ten or even up to twenty other proteins or portions thereof, or can include operably linked regulatory sequences that control expression of the coding region of the recited nucleic acid sequences, and/or can include vector sequences.

Unless specified otherwise, the left-hand end of any single-stranded polynucleotide sequence discussed herein is the 5′ end; the left-hand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA transcript that are 5′ to the 5′ end of the RNA transcript are referred to as “upstream sequences;” sequence regions on the DNA strand having the same sequence as the RNA transcript that are 3′ to the 3′ end of the RNA transcript are referred to as “downstream sequences.”

The term “control sequence” refers to a polynucleotide sequence that can affect the expression and processing of coding sequences to which it is ligated. The nature of such control sequences can depend upon the host organism. In particular embodiments, control sequences for prokaryotes can include a promoter, a ribosomal binding site, and a transcription termination sequence. For example, control sequences for eukaryotes can include promoters comprising one or a plurality of recognition sites for transcription factors, transcription enhancer sequences, and transcription termination sequence. “Control sequences” can include leader sequences and/or fusion partner sequences.

The term “vector” means any molecule or entity (e.g., nucleic acid, plasmid, bacteriophage or virus) used to transfer protein coding information into a host cell.

The term “expression vector” or “expression construct” refers to a vector that is suitable for transformation of a host cell and contains nucleic acid sequences that direct and/or control (in conjunction with the host cell) expression of one or more heterologous coding regions operatively linked thereto. An expression construct can include, but is not limited to, sequences that affect or control transcription, translation, and, if introns are present, affect RNA splicing of a coding region operably linked thereto.

As used herein, “operably linked” means that the components to which the term is applied are in a relationship that allows them to carry out their inherent functions under suitable conditions. For example, a control sequence in a vector that is “operably linked” to a protein coding sequence is ligated thereto so that expression of the protein coding sequence is achieved under conditions compatible with the transcriptional activity of the control sequences.

The term “host cell” means a cell that has been transformed, or is capable of being transformed, with a nucleic acid sequence and thereby expresses a gene of interest. The term includes the progeny of the parent cell, whether or not the progeny is identical in morphology or in genetic make-up to the original parent cell, so long as the gene of interest is present.

As used herein, the term “MHC”, unless specified otherwise, includes a reference to both MHC class I and MHC class II molecules.

The term “immune costimulatory molecule” includes cell surface molecules that act to amplify or counteract the initial activating signals provided to T cells from the T cell receptor (TCR) following its interaction with an antigen or MHC. Examples of such molecules include CD86, CD80, CD83, PD-L1, HLA-A, B, C, and HLA-DR.

The term “transfection” means the uptake of foreign or exogenous DNA by a cell, and a cell has been “transfected” when the exogenous DNA has been introduced inside the cell membrane. A number of transfection techniques are well known in the art and are disclosed herein. See, e.g., Graham et al., 1973, Virology 52:456; Sambrook et al., 2001, Molecular Cloning: A Laboratory Manual, supra; Davis et al., 1986, Basic Methods in Molecular Biology, Elsevier; Chu et al., 1981, Gene 13:197. Such techniques can be used to introduce one or more exogenous DNA moieties into suitable host cells.

The term “transformation” refers to a change in a cell's genetic characteristics, and a cell has been transformed when it has been modified to contain new DNA or RNA. For example, a cell is transformed where it is genetically modified from its native state by introducing new genetic material via transfection, transduction, or other techniques. Following transfection or transduction, the transforming DNA can recombine with that of the cell by physically integrating into a chromosome of the cell, or can be maintained transiently as an episomal element without being replicated, or can replicate independently as a plasmid. A cell is considered to have been “stably transformed” when the transforming DNA is replicated with the division of the cell.

The terms “polypeptide” or “protein” means a macromolecule having the amino acid sequence of a native protein, that is, a protein produced by a naturally-occurring and non-recombinant cell; or it is produced by a genetically-engineered or recombinant cell, and comprise molecules having the amino acid sequence of the native protein, or molecules having deletions from, additions to, and/or substitutions of one or more amino acids of the native sequence. The term also includes amino acid polymers in which one or more amino acids are chemical analogs of a corresponding naturally occurring amino acid and polymers. The terms “polypeptide” and “protein” specifically encompass agonist anti-CD40 antibodies, or sequences that have deletions from, additions to, and/or substitutions of one or more amino acid of antigen-binding protein. The term “polypeptide fragment” refers to a polypeptide that has an amino-terminal deletion, a carboxyl-terminal deletion, and/or an internal deletion as compared with the full-length native protein. Such fragments can also contain modified amino acids as compared with the native protein. In certain embodiments, fragments are about five to 500 amino acids long. For example, fragments can be at least 5, 6, 8, 10, 14, 20, 50, 70, 100, 110, 150, 200, 250, 300, 350, 400, or 450 amino acids long. Useful polypeptide fragments include immunologically functional fragments of antibodies. In the case of an agonist anti-CD40 antibody, useful fragments include but are not limited to a CDR region, a variable domain of a heavy and/or light chain, a portion of an antibody chain or just its variable region including two CDRs, and the like.

The term “isolated protein” referred means that a subject protein (1) is free of at least some other proteins with which it would normally be found, (2) is essentially free of other proteins from the same source, e.g., from the same species, (3) is expressed by a cell from a different species, (4) has been separated from at least about 50 percent of polynucleotides, lipids, carbohydrates, or other materials with which it is associated in nature, (5) is operably associated (by covalent or noncovalent interaction) with a polypeptide with which it is not associated in nature, or (6) does not occur in nature. Typically, an “isolated protein” constitutes at least about 5%, at least about 10%, at least about 25%, or at least about 50% of a given sample. Genomic DNA, cDNA, mRNA or other RNA, of synthetic origin, or any combination thereof can encode such an isolated protein. Preferably, the isolated protein is substantially free from proteins or polypeptides or other contaminants that are found in its natural environment that would interfere with its therapeutic, diagnostic, prophylactic, research or other use.

The term “amino acid” includes its normal meaning in the art.

A “variant” of a polypeptide (e.g., an antigen binding protein, or an antibody) comprises an amino acid sequence wherein one or more amino acid residues are inserted into, deleted from and/or substituted into the amino acid sequence relative to another polypeptide sequence. Variants include fusion proteins.

The term “identity” refers to a relationship between the sequences of two or more polypeptide molecules or two or more nucleic acid molecules, as determined by aligning and comparing the sequences. “Percent identity” means the percent of identical residues between the amino acids or nucleotides in the compared molecules and is calculated based on the size of the smallest of the molecules being compared. For these calculations, gaps in alignments (if any) are preferably addressed by a particular mathematical model or computer program (i.e., an “algorithm”). Methods that can be used to calculate the identity of the aligned nucleic acids or polypeptides include those described in Computational Molecular Biology, (Lesk, A. M., ed.), 1988, New York: Oxford University Press; Biocomputing Informatics and Genome Projects, (Smith, D. W., ed.), 1993, New York: Academic Press; Computer Analysis of Sequence Data, Part I, (Griffin, A. M., and Griffin, H. G., eds.), 1994, New Jersey: Humana Press; von Heinje, G., 1987, Sequence Analysis in Molecular Biology, New York: Academic Press; Sequence Analysis Primer, (Gribskov, M. and Devereux, J., eds.), 1991, New York: M. Stockton Press; and Carillo et al., 1988, SIAM J. Applied Math. 48:1073.

In calculating percent identity, the sequences being compared are typically aligned in a way that gives the largest match between the sequences. One example of a computer program that can be used to determine percent identity is the GCG program package, which includes GAP (Devereux et al, 1984, Nucl. Acid Res. 12:387; Genetics Computer Group, University of Wisconsin, Madison, Wis.). The computer algorithm GAP is used to align the two polypeptides or polynucleotides for which the percent sequence identity is to be determined. The sequences are aligned for optimal matching of their respective amino acid or nucleotide (the “matched span”, as determined by the algorithm). A gap opening penalty (which is calculated as 3× the average diagonal, wherein the “average diagonal” is the average of the diagonal of the comparison matrix being used; the “diagonal” is the score or number assigned to each perfect amino acid match by the particular comparison matrix) and a gap extension penalty (which is usually 1/10 times the gap opening penalty), as well as a comparison matrix such as PAM 250 or BLOSUM 62 are used in conjunction with the algorithm. In certain embodiments, a standard comparison matrix (see, Dayhoff et al., 1978, Atlas of Protein Sequence and Structure 5:345-352 for the PAM 250 comparison matrix; Henikoff et al., 1992, Proc. Natl. Acad. Sci. U.S.A. 89:10915-10919 for the BLOSUM 62 comparison matrix) is also used by the algorithm.

Examples of parameters that can be employed in determining percent identity for polypeptides or nucleotide sequences using the GAP program are the following:
a. Algorithm: Needleman et al., 1970, J. Mol. Biol. 48:443-453
b. Comparison matrix: BLOSUM 62 from Henikoff et al., 1992, supra
c. Gap Penalty: 12 (but with no penalty for end gaps)
d. Gap Length Penalty: 4
e. Threshold of Similarity: 0

Certain alignment schemes for aligning two amino acid sequences may result in matching of only a short region of the two sequences, and this small aligned region may have very high sequence identity even though there is no significant relationship between the two full-length sequences. Accordingly, the selected alignment method (GAP program) can be adjusted if so desired to result in an alignment that spans at least 50 or other number of contiguous amino acids of the target polypeptide.

As used herein, the twenty conventional (e.g., naturally occurring) amino acids and their abbreviations follow conventional usage. See Immunology—A Synthesis (2nd Edition, E. S. Golub and D. R. Gren, Eds., Sinauer Associates, Sunderland, Mass. (1991)), which is incorporated herein by reference for any purpose. Stereoisomers (e.g., D-amino acids) of the twenty conventional amino acids, unnatural amino acids such as −, -disubstituted amino acids, N-alkyl amino acids, lactic acid, and other unconventional amino acids can also be suitable components for polypeptides of the present invention. Examples of unconventional amino acids include: 4-hydroxyproline, -carboxyglutamate, —N,N,N-trimethyllysine, —N-acetyllysine, 0-phosphoserine, N-acetylserine, N-formylmethionine, 3-methylhistidine, 5-hydroxylysine, —N-methylarginine, and other similar amino acids and imino acids (e.g., 4-hydroxyproline). In the polypeptide notation used herein, the left-hand direction is the amino terminal direction and the right-hand direction is the carboxy-terminal direction, in accordance with standard usage and convention.

Similarly, unless specified otherwise, the left-hand end of single-stranded polynucleotide sequences is the 5′ end; the left-hand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA and which are 5′ to the 5′ end of the RNA transcript are referred to as “upstream sequences”; sequence regions on the DNA strand having the same sequence as the RNA and which are 3′ to the 3′ end of the RNA transcript are referred to as “downstream sequences.”

Conservative amino acid substitutions can encompass non-naturally occurring amino acid residues, which are typically incorporated by chemical peptide synthesis rather than by synthesis in biological systems. These include peptidomimetics and other reversed or inverted forms of amino acid moieties.

The entire disclosures of all publications (including patents, patent applications, journal articles, laboratory manuals, books, or other documents) cited herein are hereby incorporated by reference. No admission is made that any of the references constitute prior art or are part of the common general knowledge of those working in the field to which this invention relates.

Naturally occurring residues can be divided into classes based on common side chain properties:
a. hydrophobic: norleucine, Met, Ala, Val, Leu, Ile;
b. neutral hydrophilic: Cys, Ser, Thr, Asn, Gln;
c. acidic: Asp, Glu;
d. basic: His, Lys, Arg;
e. residues that influence chain orientation: Gly, Pro; and
f. aromatic: Trp, Tyr, Phe.

For example, non-conservative substitutions can involve the exchange of a member of one of these classes for a member from another class. Such substituted residues can be introduced, for example, into regions of a human antibody that are homologous with non-human antibodies, or into the non-homologous regions of the molecule.

In making changes to the agonist anti-CD40 antibodies, according to certain embodiments, the hydropathic index of amino acids can be considered. Each amino acid has been assigned a hydropathic index on the basis of its hydrophobicity and charge characteristics. They are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is understood in the art. Kyte et al., J. Mol. Biol., 157:105-131 (1982). It is known that certain amino acids can be substituted for other amino acids having a similar hydropathic index or score and still retain a similar biological activity. In making changes based upon the hydropathic index, in certain embodiments, the substitution of amino acids whose hydropathic indices are within ±2 is included. In certain embodiments, those which are within ±1 are included, and in certain embodiments, those within ±0.5 are included.

It is also understood in the art that the substitution of like amino acids can be made effectively on the basis of hydrophilicity, particularly where the biologically functional protein or peptide thereby created is intended for use in immunological embodiments, as in the present case. In certain embodiments, the greatest local average hydrophilicity of a protein, as governed by the hydrophilicity of its adjacent amino acids, correlates with its immunogenicity and antigenicity, i.e., with a biological property of the protein.

The following hydrophilicity values have been assigned to these amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5) and tryptophan (−3.4). In making changes based upon similar hydrophilicity values, in certain embodiments, the substitution of amino acids whose hydrophilicity values are within ±2 is included, in certain embodiments, those which are within ±1 are included, and in certain embodiments, those within ±0.5 are included. One can also identify epitopes from primary amino acid sequences on the basis of hydrophilicity. These regions are also referred to as “epitopic core regions.”

Exemplary amino acid substitutions are set forth in Table 2.

TABLE 2
Amino Acid Substitutions

| Original Residues | Exemplary Substitutions | Preferred Substitutions |
|---|---|---|
| Ala | Val, Leu, Ile | Val |
| Arg | Lys, Gln, Asn | Lys |
| Asn | Gln | Gln |
| Asp | Glu | Glu |
| Cys | Ser, Ala | Ser |
| Gln | Asn | Asn |
| Glu | Asp | Asp |
| Gly | Pro, Ala | Ala |
| His | Asn, Gln, Lys, Arg | Arg |
| Ile | Leu, Val, Met, Ala, | Leu |
| | Phe, Norleucine | |
| Leu | Norleucine, Ile, | Ile |
| | Val, Met, Ala, Phe | |
| Lys | Arg, 1,4 Diamino-butyric | Arg |
| | Acid, Gln, Asn | |
| Met | Leu, Phe, Ile | Leu |
| Phe | Leu, Val, Ile, Ala, | Leu |
| | Tyr | |
| Pro | Ala | Gly |
| Ser | Thr, Ala, Cys | Thr |
| Thr | Ser | Ser |
| Trp | Tyr, Phe | Tyr |
| Tyr | Trp, Phe, Thr, Ser | Phe |
| Val | Ile, Met, Leu, Phe, | Leu |
| | Ala, Norleucine | |

The term “derivative” refers to a molecule that includes a chemical modification other than an insertion, deletion, or substitution of amino acids (or nucleic acids). In certain embodiments, derivatives comprise covalent modifications, including, but not limited to, chemical bonding with polymers, lipids, or other organic or inorganic moieties. In certain embodiments, a chemically modified antigen binding protein can have a greater circulating half-life than an antigen binding protein that is not chemically modified. In certain embodiments, a chemically modified antigen binding protein can have improved targeting capacity for desired cells, tissues, and/or organs. In some embodiments, a derivative antigen binding protein is covalently modified to include one or more water soluble polymer attachments, including, but not limited to, polyethylene glycol, polyoxyethylene glycol, or polypropylene glycol. See, e.g., U.S. Pat. Nos. 4,640,835, 4,496,689, 4,301,144, 4,670,417, 4,791,192 and 4,179,337. In certain embodiments, a derivative antigen binding protein comprises one or more polymers, including, but not limited to, monomethoxy-polyethylene glycol, dextran, cellulose, or other carbohydrate based polymers, poly-(N-vinyl pyrrolidone)-polyethylene glycol, propylene glycol homopolymers, a polypropylene oxide/ethylene oxide co-polymer, polyoxyethylated polyols (e.g., glycerol) and polyvinyl alcohol, as well as mixtures of such polymers.

In certain embodiments, a derivative is covalently modified with polyethylene glycol (PEG) subunits. In certain embodiments, one or more water-soluble polymer is bonded at one or more specific position, for example at the amino terminus, of a derivative. In certain embodiments, one or more water-soluble polymers is randomly attached to one or more side chains of a derivative. In certain embodiments, PEG is used to improve the therapeutic capacity for an antigen binding protein. In certain embodiments, PEG is used to improve the therapeutic capacity for a humanized antibody. Certain such methods are discussed, for example, in U.S. Pat. No. 6,133,426, which is hereby incorporated by reference for any purpose.

Peptide analogs are commonly used in the pharmaceutical industry as non-peptide drugs with properties analogous to those of the template peptide. These types of non-peptide compound are termed “peptide mimetics” or “peptidomimetics.” Fauchere, J., Adv. Drug Res., 15:29 (1986); Veber & Freidinger, TINS, p. 392 (1985); and Evans et al., J. Med. Chem., 30:1229 (1987), which are incorporated herein by reference for any purpose. Such compounds are often developed with the aid of computerized molecular modeling. Peptide mimetics that are structurally similar to therapeutically useful peptides can be used to produce a similar therapeutic or prophylactic effect. Generally, peptidomimetics are structurally similar to a paradigm polypeptide (i.e., a polypeptide that has a biochemical property or pharmacological activity), such as human antibody, but have one or more peptide linkages optionally replaced by a linkage selected from: —CH₂NH—, —CH₂S—, —CH₂—CH₂—, —CH═CH-(cis and trans), —COCH₂—CH(OH)CH₂—, and —CH₂SO—, by methods well known in the art. Systematic substitution of one or more amino acids of a consensus sequence with a D-amino acid of the same type (e.g., D-lysine in place of L-lysine) can be used in certain embodiments to generate more stable peptides. In addition, constrained peptides comprising a consensus sequence or a substantially identical consensus sequence variation can be generated by methods known in the art (Rizo and Gierasch, Ann. Rev. Biochem., 61:387 (1992), incorporated herein by reference for any purpose); for example, by adding internal cysteine residues capable of forming intramolecular disulfide bridges which cyclize the peptide.

The term “naturally occurring” as used throughout the specification in connection with biological materials such as polypeptides, nucleic acids, host cells, and the like, refers to materials which are found in nature or a form of the materials that is found in nature.

The term “Agonist” refers to a compound that, in combination with a receptor, can produce a cellular response. An agonist may be a ligand that directly binds to the receptor. Alternatively, an agonist may combine with a receptor indirectly by, for example, (a) forming a complex with another molecule that directly binds to the receptor, or (b) otherwise resulting in the modification of another compound so that the other compound directly binds to the receptor. An agonist may be referred to as an agonist of a particular receptor or family of receptors (e.g., a TNF or TNFR agonist).

The term “immunologically functional fragment” (or simply “fragment”) of an antibody or immunoglobulin chain (heavy or light chain) antibody is a species of antibody comprising a portion (regardless of how that portion is obtained or synthesized) of an antibody that lacks at least some of the amino acids present in a full-length chain but which is still capable of specifically performing as an agonist for CD40. These biologically active fragments can be produced by recombinant DNA techniques or can be produced by enzymatic or chemical cleavage of antigen binding proteins, including intact antibodies. Immunologically functional immunoglobulin fragments include, but are not limited to, Fab, a diabody (heavy chain variable domain on the same polypeptide as a light chain variable domain, connected via a short peptide linker that is too short to permit pairing between the two domains on the same chain), Fab′, F(ab′)2, Fv, domain antibodies and single-chain antibodies, and can be derived from any mammalian source, including but not limited to human, mouse, rat, camelid or rabbit. It is further contemplated that a functional portion of the agonist anti-CD40 antibody disclosed herein, for example, one or more CDRs, could be covalently bound to a second protein or to a small molecule to create a therapeutic agent directed to a particular target in the body, possessing bifunctional therapeutic properties, or having a prolonged serum half-life. As will be appreciated by one of skill in the art, an agonist anti-CD40 antibody can include nonprotein components.

In certain embodiments, the polypeptide structure of the agonist anti-CD40 antibody is based on antibodies, including, but not limited to, monoclonal antibodies, bispecific antibodies, minibodies, domain antibodies, synthetic antibodies (sometimes referred to herein as “antibody mimetics”), chimeric antibodies, humanized antibodies, antibody fusions (sometimes referred to herein as “antibody conjugates”), and fragments thereof, respectively.

An “Fc” region comprises two heavy chain fragments comprising the C_{H}1 and C_{H}2 domains of an antibody. The two heavy chain fragments are held together by two or more disulfide bonds and by hydrophobic interactions of the C_{H}3 domains.

A “Fab fragment” comprises one light chain and the C_{H}1 and variable regions of one heavy chain. The heavy chain of a Fab molecule cannot form a disulfide bond with another heavy chain molecule.

A “Fab′ fragment” comprises one light chain and a portion of one heavy chain that contains the VH domain and the C_{H}1 domain and also the region between the C_{H}1 and C_{H}2 domains, such that an interchain disulfide bond can be formed between the two heavy chains of two Fab′ fragments to form an F(ab′)2 molecule.

The “Fv region” comprises the variable regions from both the heavy and light chains but lacks the constant regions.

“Single-chain antibodies” are Fv molecules in which the heavy and light chain variable regions have been connected by a flexible linker to form a single polypeptide chain, which forms an antigen binding region. Single chain antibodies are discussed in detail in International Patent Application Publication No. WO 88/01649 and U.S. Pat. Nos. 4,946,778 and 5,260,203, the disclosures of which are incorporated by reference.

A “domain antibody” is an immunologically functional immunoglobulin fragment containing only the variable region of a heavy chain or the variable region of a light chain. In some instances, two or more V_{H} regions are covalently joined with a peptide linker to create a bivalent domain antibody. The two V_{H} regions of a bivalent domain antibody can target the same or different antigens.

As used herein, “substantially pure” means that the described species of molecule is the predominant species present, that is, on a molar basis it is more abundant than any other individual species in the same mixture. In certain embodiments, a substantially pure molecule is a composition wherein the object species comprises at least 50% (on a molar basis) of all macromolecular species present. In other embodiments, a substantially pure composition will comprise at least 80%, 85%, 90%, 95%, or 99% of all macromolecular species present in the composition. In other embodiments, the object species is purified to essential homogeneity wherein contaminating species cannot be detected in the composition by conventional detection methods and thus the composition consists of a single detectable macromolecular species.

The term “agent” is used herein to denote a chemical compound, a mixture of chemical compounds, a biological macromolecule, or an extract made from biological materials.

As used herein, the terms “label” or “labeled” refers to incorporation of a detectable marker, e.g., by incorporation of a radiolabeled amino acid or attachment to a polypeptide of biotin moieties that can be detected by marked avidin (e.g., streptavidin containing a fluorescent marker or enzymatic activity that can be detected by optical or colorimetric methods). In certain embodiments, the label or marker can also be therapeutic. Various methods of labeling polypeptides and glycoproteins are known in the art and can be used. Examples of labels for polypeptides include, but are not limited to, the following: radioisotopes or radionuclides (e.g., ³H, ¹⁴C, ¹⁵N, ³⁵S, ⁹⁰Y, ⁹⁹Tc, ¹¹¹In, ¹²⁵I, ¹³¹I), fluorescent labels (e.g., FITC, rhodamine, lanthanide phosphors), enzymatic labels (e.g., horseradish peroxidase, 3-galactosidase, luciferase, alkaline phosphatase), chemiluminescent, biotinyl groups, predetermined polypeptide epitopes recognized by a secondary reporter (e.g., leucine zipper pair sequences, binding sites for secondary antibodies, metal binding domains, epitope tags). In certain embodiments, labels are attached by spacer arms of various lengths to reduce potential steric hindrance.

The term “therapeutically effective amount” refers to the amount of an agonist anti-CD40 antibody determined to produce a therapeutic response in a mammal (preferably a human). Such therapeutically effective amounts are readily ascertained by one of ordinary skill in the art.

The term “pharmaceutical agent composition” (or agent or drug) as used herein refers to a chemical compound, composition, agent or drug capable of inducing a desired therapeutic effect when properly administered to a patient. It does not necessarily require more than one type of ingredient.

Throughout this specification, unless the context requires otherwise, the word “comprise”, or variations such as “comprises” or “comprising”, will be understood to imply the inclusion of a stated integer or group of integers but not the exclusion of any other integer or group of integers.

Other definitions for selected terms used herein may be found within the detailed description of the invention and apply throughout. Unless otherwise defined, all other scientific and technical terms used herein have the same meaning as commonly understood to one of ordinary skill in the art to which the invention belongs.

The invention described herein may include one or more range of values (for example, size, displacement and field strength etc.). A range of values will be understood to include all values within the range, including the values defining the range, and values adjacent to the range that lead to the same or substantially the same outcome as the values immediately adjacent to that value which defines the boundary to the range. For example, a person skilled in the field will understand that a 10% variation in upper or lower limits of a range can be totally appropriate and is encompassed by the invention. More particularly, the variation in upper or lower limits of a range will be 5% or as is commonly recognised in the art, whichever is greater.

Throughout this specification relative language such as the words ‘about’ and ‘approximately’ are used. This language seeks to incorporate at least 10% variability to the specified number or range. That variability may be plus 10% or negative 10% of the particular number specified.

### Embodiments

A. Agonist Anti-CD40 Antibodies

The agonist anti-CD40 antibodies of the invention can modulate at least one or more of the following functions when administered in an effective amount:
a. increasing antigen presentation by APCs (including macrophages, DCs, and B cells); or
b. enhancing the expression of MHC and/or immune costimulatory molecules (such as CD86, CD80, CD83, PD-L1, HLA-A, B, C, or HLA-DR); or
c. stimulating the production of pro-inflammatory cytokines (such as IL-12, IL-1β, IL-6, IL-10, IL-12p40, IL-12p70, IL-23 and IFN-γ); or
d. inducing T cell activation; or
e. mimicking the signal of CD40L and substituting for the function of CD4+ lymphocytes; or
f. overcoming T cell tolerance in tumor-bearing animals; or
g. evoking effective cytotoxic T cell responses; or
h. enhancing the efficacy of anti-tumor vaccines; or
i. rendering tumor vasculature more permissive to immune infiltrates

In some embodiments, the agonist anti-CD40 antibodies provided are polypeptides which comprise one or more CDRs, as described herein. In some agonist anti-CD40 antibodies, the CDRs are embedded into a “framework” region, which orients the CDR(s) such that the proper binding properties of the CDR(s) is achieved.

The antibodies of the invention disclosed herein have a variety of utilities. They can be used in a variety of therapeutic applications, as explained herein. For example, in some embodiments the agonist anti-CD40 antibodies are useful for treating malignant conditions, including but not limited to when administered with other agents, such as IL-2. Other uses for the agonist anti-CD40 antibodies include, for example, diagnosis of disease states or conditions and screening assays to determine the presence or absence of CD40. Some of the agonist anti-CD40 antibodies described herein are also useful in treating consequences, symptoms, and/or the pathology associated with increasing antigen presentation.

In one form, the invention comprises an isolated agonist anti-CD40 antibody or fragment thereof which comprises (i) a V_{H} chain comprising three CDRs and (ii) a V_{L} chain comprising three CDRs, wherein one or more heavy chain complementary determining regions (CDRHs) is selected from the group consisting of:
a) a CDRH1 sequence comprising SEQ ID NO:1;
b) a CDRH2 sequence comprising SEQ ID NO:2; or
c) a CDRH2 sequence comprising SEQ ID NO:2 that contains one or two amino acid substitutions, deletions or insertions.

In some embodiments, the agonist anti-CD40 antibodies that are provided comprise one or more CDRs (e.g., 1, 2, 3, 4, 5 or 6 CDRs). In some embodiments, the agonist anti-CD40 antibody comprises (a) a polypeptide structure and (b) one or more CDRs that are inserted into and/or joined to the polypeptide structure. The polypeptide structure can take a variety of different forms. For example, it can be, or comprise, the framework of a naturally occurring antibody, or fragment or variant thereof, or can be completely synthetic in nature. Examples of various polypeptide structures are further described below.

In certain embodiments, the polypeptide structure of the agonist anti-CD40 antibody is an antibody or is derived from an antibody, including, but not limited to, monoclonal antibodies, bispecific antibodies, minibodies, domain antibodies, synthetic antibodies (sometimes referred to herein as “antibody mimetics”), chimeric antibodies, humanized antibodies, antibody fusions (sometimes referred to as “antibody conjugates”), and portions or fragments of each, respectively. In some instances, the agonist anti-CD40 antibody is an immunological fragment of an antibody (e.g., a Fab, a Fab′, a F(ab′)2, or a scFv). The various structures are further described and defined herein.

In an embodiment of the first form of the invention, the isolated agonist anti-CD40 antibody additionally comprises:
a) a CDRH3 sequence comprising SEQ ID NO:3;
b) a CDRL1 sequence comprising SEQ ID NO:4;
c) a CDRL2 sequence comprising SEQ ID NO:5; or
d) a CDRL3 sequence comprising SEQ ID NO:6

In a further embodiment of the first form of the invention, the isolated agonist anti-CD40 antibody comprises:

### A:

a) a CDRH1 sequence comprising SEQ ID NO:1; or
b) a CDRH2 sequence comprising SEQ ID NO:2; and

### B:

c) a CDRH3 sequence comprising SEQ ID NO:3;
d) a CDRL1 sequence comprising SEQ ID NO:4;
e) a CDRL2 sequence comprising SEQ ID NO:5; or
f) a CDRL3 sequence comprising SEQ ID NO:6

In another embodiment of the first form of the invention the isolated agonist anti-CD40 antibody comprises:
a) a CDRH1 sequence comprising SEQ ID NO:1;
b) a CDRH2 sequence comprising SEQ ID NO:2;
c) a CDRH3 sequence comprising SEQ ID NO:3;
d) a CDRL1 sequence comprising SEQ ID NO:4;
e) a CDRL2 sequence comprising SEQ ID NO:5; and
f) a CDRL3 sequence comprising SEQ ID NO:6

In another embodiment of the first form of the invention the isolated agonist anti-CD40 antibody comprises a heavy chain variable region of SEQ ID NO: 7. In a further embodiment of the first form of the invention the isolated agonist anti-CD40 antibody comprises a light chain variable region of SEQ ID NO: 8. In a further embodiment of the first form of the invention, the isolated agonist anti-CD40 antibody comprises a heavy chain variable region of SEQ ID NO: 7 and a light chain variable region of SEQ ID NO: 8. SEQ ID NO:7 comprises the CDRH1, CDRH2, and CDRH3 sequences of SEQ ID NO:1, SEQ ID NO:2 and SEQ ID NO:3. SEQ ID NO:8 comprises the CDRL1, CDRL2, and CDRL3 sequences of SEQ ID NO:4, SEQ ID NO:5 and SEQ ID NO:6. In some embodiments, an antibody comprising the heavy chain variable region of SEQ ID NO: 7 and light chain variable region of SEQ ID NO: 8 is known as SVX-3001.

In an embodiment, the isolated agonist anti-CD40 antibody is a monoclonal antibody, a polyclonal antibody, a recombinant antibody, a human antibody, a humanized antibody, a chimeric antibody, a multispecific antibody, or an antibody fragment thereof.

In an embodiment, the isolated agonist anti-CD40 antibody is a Fab fragment, a Fab′ fragment, a F(ab′)2 fragment, a Fv fragment, a diabody, or a single chain antibody molecule.

In an embodiment, the isolated agonist anti-CD40 antibody is a humanized anti-human antibody.

In an embodiment, the isolated agonist anti-CD40 antibody is a monoclonal antibody.

In an embodiment, the isolated agonist anti-CD40 antibody is of the IgG1-, IgG2- IgG3- or IgG4-type. Preferably, the isolated agonist anti-CD40 antibody is of the IgG1 type.

In a particularly preferred embodiment, the isolated agonist anti-CD40 antibody comprises the heavy chain sequence of SEQ ID NO:19 and/or the light chain sequence of SEQ ID NO: 20.

In an embodiment, the isolated agonist anti-CD40 antibody is coupled to a labeling group.

Other antibodies that are provided are variants of the agonist anti-CD40 antibodies listed above formed by combination or subparts of the variable heavy and variable light chains shown in SEQ ID NO: 7 and 8, and comprise variable light and/or variable heavy chains that each have at least 50%, 50-60, 60-70, 70-80%, 80-85%, 85-90%, 90-95%, 95-97%, 97-99%, or above 99% identity to the amino acid sequences of the sequences in SEQ ID NO: 7 and 8 (either the entire sequence or a subpart of the sequence, e.g., one or more CDR). In some instances, such antibodies include at least one heavy chain and one light chain, whereas in other instances the variant forms contain two identical light chains and two identical heavy chains (or subparts thereof).

In certain embodiments, an agonist anti-CD40 antibodies comprises a heavy chain comprising a variable region comprising an amino acid sequence at least 90% identical to an amino acid sequence of SEQ ID NO: 7. In certain embodiments, an agonist anti-CD40 antibodies comprises a heavy chain comprising a variable region comprising an amino acid sequence at least 95% identical to an amino acid sequence of SEQ ID NO: 7. In certain embodiments, an agonist anti-CD40 antibodies comprises a heavy chain comprising a variable region comprising an amino acid sequence at least 99% identical to an amino acid sequence to SEQ ID NO: 7.

In certain embodiments, an agonist anti-CD40 antibodies comprises a heavy chain comprising an amino acid sequence at least 90% identical to an amino acid sequence of SEQ ID NO: 19. In certain embodiments, an agonist anti-CD40 antibodies comprises a heavy chain comprising an amino acid sequence at least 95% identical to an amino acid sequence of SEQ ID NO: 19 In certain embodiments, an agonist anti-CD40 antibodies comprises a heavy chain comprising an amino acid sequence at least 99% identical to an amino acid sequence to SEQ ID NO: 19.

In some embodiments, the agonist anti-CD40 antibodies comprise a sequence that is at least 90%, 90-95%, and/or 95-99% identical to one or more CDRs from the CDRs in at least one of sequences of SEQ ID NO: 1 to 3 and 4 to 6. In some embodiments, 1, 2, 3, 4, 5, or 6 CDRs (each being at least 90%, 90-95%, and/or 95-99% identical to the above sequences) are present.

In certain embodiments, agonist anti-CD40 antibodies comprise a light chain comprising a variable region comprising an amino acid sequence at least 90% identical to SEQ ID NO:8. In certain embodiments, an agonist anti-CD40 antibodies comprises a light chain comprising a variable region comprising an amino acid sequence at least 95% identical to SEQ ID NO: 8. In certain embodiments, the agonist anti-CD40 antibodies comprise a light chain comprising a variable region comprising an amino acid sequence at least 99% identical to SEQ ID NO: 8.

In certain embodiments, agonist anti-CD40 antibodies comprise a light chain comprising an amino acid sequence at least 90% identical to SEQ ID NO:20. In certain embodiments, an agonist anti-CD40 antibodies comprises a light chain comprising an amino acid sequence at least 95% identical to SEQ ID NO: 20. In certain embodiments, the agonist anti-CD40 antibodies comprise a light chain comprising an amino acid sequence at least 99% identical to SEQ ID NO: 20.

In an embodiment, the isolated agonist anti-CD40 antibody enhances CD40 activity.

B. Manufacture of Agonist Anti-CD40 Antibodies

In an embodiment, the invention comprises a method of making the agonist anti-CD40 antibody as described herein, comprising the step of preparing said agonist anti-CD40 antibody from a host cell that secretes said agonist anti-CD40 antibody.

Generally, fully monoclonal agonist antibodies for CD40 can be produced as follows. Mice containing immunoglobulin genes are immunized with the CD40 of interest, lymphatic cells (such as B-cells) from the mice that express antibodies are obtained. Such recovered cells are fused with a myeloid-type cell line to prepare immortal hybridoma cell lines, and such hybridoma cell lines are screened and selected to identify hybridoma cell lines that produce antibodies specific to the antigen of interest. In certain embodiments, the production of a hybridoma cell line that produces agonist antibodies specific to CD40 is provided.

In certain embodiments, a phage display technique is used to generate monoclonal antibodies. In certain embodiments, such techniques produce monoclonal antibodies. In certain embodiments, a polynucleotide encoding a single Fab or Fv antibody fragment is expressed on the surface of a phage particle. See, e.g., Hoogenboom et al., J. Mol. Biol., 227: 381 (1991); Marks et al., J Mol Biol 222: 581 (1991); U.S. Pat. No. 5,885,793. In certain embodiments, phage are “screened” to identify those antibody fragments having affinity for target. Thus, certain such processes mimic immune selection through the display of antibody fragment repertoires on the surface of filamentous bacteriophage, and subsequent selection of phage by their binding to target. In certain such procedures, high affinity functional antibody fragments are isolated. In certain such embodiments, a complete repertoire of antibody genes is created by cloning naturally rearranged V genes from peripheral blood lymphocytes. See, e.g., Mullinax et aL, Proc Natl Acad Sci (USA), 87: 8095-8099 (1990).

According to certain embodiments, antibodies of the invention are prepared through the utilization of a transgenic mouse that has a substantial portion of the antibody producing genome inserted but that is rendered deficient in the production of endogenous, murine antibodies. Such mice, then, are capable of producing immunoglobulin molecules and antibodies and are deficient in the production of murine immunoglobulin molecules and antibodies. Technologies utilized for achieving this result are disclosed in the patents, applications and references disclosed in the specification, herein. In certain embodiments, one can employ methods such as those disclosed in PCT Published Application No. WO 98/24893 or in Mendez et al., Nature Genetics, 15:146-156 (1997), which are hereby incorporated by reference for any purpose.

In certain embodiments, agonist antibodies specific to CD40 are produced by exposing splenocytes (B or T cells) to an antigen in vitro, and then reconstituting the exposed cells in an immunocompromised mouse, e.g. SCID or nod/SCID. See, e.g., Brams et al., J. Immunol. 160: 2051-2058 (1998); Carballido et al., Nat. Med., 6: 103-106 (2000). In certain such approaches, engraftment of fetal tissue into SCID mice (SCID-hu) results in long-term hematopoiesis and human T-cell development. See, e.g., McCune et al., Science, 241:1532-1639 (1988); Ifversen et al., Sem. Immunol., 8:243-248 (1996). In certain instances, humoral immune response in such chimeric mice is dependent on co-development of T-cells in the animals. See, e.g., Martensson et al., Immunol., 83:1271-179 (1994). In certain approaches, peripheral blood lymphocytes are transplanted into SCID mice. See, e.g., Mosier et al., Nature, 335:256-259 (1988). In certain such embodiments, when such transplanted cells are treated either with a priming agent, such as Staphylococcal Enterotoxin A (SEA), higher levels of B cell production is detected. See, e.g., Martensson et al., Immunol., 84: 224-230 (1995); Murphy et al., Blood, 86:1946-1953 (1995).

As will be appreciated, antibodies can be expressed in cell lines other than hybridoma cell lines. Sequences encoding particular antibodies can be used to transform a suitable mammalian host cell. Transformation can be by any known method for introducing polynucleotides into a host cell, including, for example packaging the polynucleotide in a virus (or into a viral vector) and transducing a host cell with the virus (or vector) or by transfection procedures known in the art, as exemplified by U.S. Pat. Nos. 4,399,216, 4,912,040, 4,740,461, and 4,959,455 (which patents are hereby incorporated herein by reference). The transformation procedure used depends upon the host to be transformed. Methods for introducing heterologous polynucleotides into mammalian cells are well known in the art and include dextran-mediated transfection, calcium phosphate precipitation, polybrene mediated transfection, protoplast fusion, electroporation, encapsulation of the polynucleotide(s) in liposomes, and direct microinjection of the DNA into nuclei.

Mammalian cell lines available as hosts for expression are well known in the art and include many immortalized cell lines available from the American Type Culture Collection (ATCC), including but not limited to Chinese hamster ovary (CHO) cells, HeLa cells, baby hamster kidney (BHK) cells, monkey kidney cells (COS), human hepatocellular carcinoma cells (e.g., Hep G2), human epithelial kidney 293 cells, and a number of other cell lines. Cell lines of particular preference are selected through determining which cell lines have high expression levels and produce agonist antibodies specific to CD40.

In certain embodiments, agonist CD40 antibodies comprise an immunoglobulin molecule of at least one of the IgG1, IgG2, IgG3, IgG4, Ig E, IgA, IgD, and IgM isotype. In certain embodiments, agonist CD40 antibodies comprise a human kappa light chain and/or a human heavy chain. In certain embodiments, the heavy chain is of the IgG1, IgG2, IgG3, IgG4, IgE, IgA, IgD, or IgM isotype. In certain embodiments, agonist CD40 antibodies have been cloned for expression in mammalian cells. In certain embodiments, agonist CD40 antibodies comprise a constant region other than any of the constant regions of the IgG1, IgG2, IgG3, IgG4, IgE, IgA, IgD, and IgM isotype.

In certain embodiments, agonist CD40 antibodies comprise a human lambda light chain and a human IgG2 heavy chain. In certain embodiments, agonist CD40 antibodies comprise a human lambda light chain and a human IgG4 heavy chain. In certain embodiments, agonist CD40 antibodies comprise a human lambda light chain and a human IgG1 heavy chain. In certain embodiments, agonist CD40 antibodies comprise a human lambda light chain and a human IgG3, IgE, IgA, IgD or IgM heavy chain. In other embodiments, agonist CD40 antibodies comprise a human kappa light chain and a human IgG2 heavy chain. In certain embodiments, agonist CD40 antibodies comprise a human kappa light chain and a human IgG4 heavy chain. In certain embodiments, agonist CD40 antibodies comprise a human kappa light chain and a human IgG1 heavy chain. In certain embodiments, agonist CD40 antibodies comprise a human kappa light chain and a human IgG3, IgE, IgA, IgD or IgM heavy chain. In certain embodiments, agonist CD40 antibodies comprise variable regions of antibodies ligated to a constant region that is neither the constant region for the IgG2 isotype, nor the constant region for the IgG4 isotype. Preferably, the agonist CD40 antibodies comprise a human IgG1 heavy and light chain. In certain embodiments, agonist CD40 antibodies have been cloned for expression in mammalian cells.

In certain embodiments, conservative modifications to the heavy and light chains of antibodies from at least one of the hybridoma lines described herein will produce agonist CD40 antibodies having functional and chemical characteristics similar to those of the antibodies from the hybridoma lines. In contrast, in certain embodiments, substantial modifications in the functional and/or chemical characteristics of agonist CD40 antibodies can be accomplished by selecting substitutions in the amino acid sequence of the heavy and light chains that differ significantly in their effect on maintaining (a) the structure of the molecular backbone in the area of the substitution, for example, as a sheet or helical conformation, (b) the charge or hydrophobicity of the molecule at the target site, or (c) the bulk of the side chain.

For example, a “conservative amino acid substitution” can involve a substitution of a native amino acid residue with a nonnative residue such that there is little or no effect on the polarity or charge of the amino acid residue at that position. Furthermore, any native residue in the polypeptide can also be substituted with alanine, as has been previously described for “alanine scanning mutagenesis.”

Desired amino acid substitutions (whether conservative or non-conservative) can be determined by those skilled in the art at the time such substitutions are desired. In certain embodiments, amino acid substitutions can be used to identify important residues of agonist CD40 antibodies, or to increase or decrease the agonist activity of agonist CD40 antibodies as described herein.

In certain embodiments, agonist CD40 antibodies comprise one or more polypeptides. In certain embodiments, any of a variety of expression vector/host systems can be utilized to express polynucleotide molecules encoding polypeptides comprising one or more agonist anti-CD40 antibody components or the agonist anti-CD40 antibody itself. Such systems include, but are not limited to, microorganisms, such as bacteria transformed with recombinant bacteriophage, plasmid, or cosmid DNA expression vectors; yeast transformed with yeast expression vectors; insect cell systems infected with virus expression vectors (e.g., baculovirus); plant cell systems transfected with virus expression vectors (e.g., cauliflower mosaic virus, CaMV, tobacco mosaic virus, TMV) or transformed with bacterial expression vectors (e.g., Ti or pBR322 plasmid); or animal cell systems.

In certain embodiments, a polypeptide comprising one or more agonist anti-CD40 antibody components or the agonist anti-CD40 antibody itself is recombinantly expressed in yeast. Certain such embodiments use commercially available expression systems, e.g., the Pichia Expression System (Invitrogen, San Diego, Calif.), following the manufacturer's instructions. In certain embodiments, such a system relies on the pre-pro-alpha sequence to direct secretion. In certain embodiments, transcription of the insert is driven by the alcohol oxidase (AOX1) promoter upon induction by methanol.

In certain embodiments, a secreted polypeptide comprising one or more agonist anti-CD40 antibody components or the agonist anti-CD40 antibody itself is purified from yeast growth medium. In certain embodiments, the methods used to purify a polypeptide from yeast growth medium is the same as those used to purify the polypeptide from bacterial and mammalian cell supernatants.

In certain embodiments, a nucleic acid encoding a polypeptide comprising one or more agonist anti-CD40 antibody components or the agonist anti-CD40 antibody itself is cloned into a baculovirus expression vector, such as pVL1393 (PharMingen, San Diego, Calif.). In certain embodiments, such a vector can be used according to the manufacturer's directions (PharMingen) to infect Spodoptera frugiperda cells in sF9 protein-free media and to produce recombinant polypeptide. In certain embodiments, a polypeptide is purified and concentrated from such media using a heparin-Sepharose column (Pharmacia).

In certain embodiments, a polypeptide comprising one or more agonist anti-CD40 antibody components or the agonist anti-CD40 antibody itself is expressed in an insect system. Certain insect systems for polypeptide expression are well known to those of skill in the art. In one such system, Autographa californica nuclear polyhedrosis virus (AcNPV) is used as a vector to express foreign genes in Spodoptera frugiperda cells or in Trichoplusia larvae. In certain embodiments, a nucleic acid molecule encoding a polypeptide can be inserted into a nonessential gene of the virus, for example, within the polyhedrin gene, and placed under control of the promoter for that gene. In certain embodiments, successful insertion of a nucleic acid molecule will render the nonessential gene inactive. In certain embodiments, that inactivation results in a detectable characteristic. For example, inactivation of the polyhedrin gene results in the production of virus lacking coat protein.

In certain embodiments, recombinant viruses can be used to infect S. frugiperda cells or Trichoplusia larvae. See, e.g., Smith et al., J. Virol., 46: 584 (1983); Engelhard et al., Proc. Nat. Acad. Sci. (USA), 91: 3224-7 (1994).

In certain embodiments, polypeptides comprising one or more agonist anti-CD40 antibody components or the agonist anti-CD40 antibody itself made in bacterial cells are produced as insoluble inclusion bodies in the bacteria. In certain embodiments, host cells comprising such inclusion bodies are collected by centrifugation; washed in 0.15 M NaCl, 10 mM Tris, pH 8, 1 mM EDTA; and treated with 0.1 mg/ml lysozyme (Sigma, St. Louis, Mo.) for 15 minutes at room temperature. In certain embodiments, the lysate is cleared by sonication, and cell debris is pelleted by centrifugation for 10 minutes at 12,000×g. In certain embodiments, the polypeptide-containing pellet is resuspended in 50 mM Tris, pH 8, and 10 mM EDTA; layered over 50% glycerol; and centrifuged for 30 minutes at 6000×g. In certain embodiments, that pellet can be resuspended in standard phosphate buffered saline solution (PBS) free of Mg⁺⁺ and Ca⁺⁺. In certain embodiments, the polypeptide is further purified by fractionating the resuspended pellet in a denaturing SDS polyacrylamide gel (See, e.g., Sambrook et al., supra). In certain embodiments, such a gel can be soaked in 0.4 M KCl to visualize the protein, which can be excised and electroeluted in gel-running buffer lacking SDS. According to certain embodiments, a Glutathione-S-Transferase (GST) fusion protein is produced in bacteria as a soluble protein. In certain embodiments, such GST fusion protein is purified using a GST Purification Module (Pharmacia).

In certain embodiments, it is desirable to “refold” certain polypeptides, e.g., polypeptides comprising one or more agonist anti-CD40 antibody components or the agonist anti-CD40 antibody itself. In certain embodiments, such polypeptides are produced using certain recombinant systems discussed herein. In certain embodiments, polypeptides are “refolded” and/or oxidized to form desired tertiary structure and/or to generate disulfide linkages. In certain embodiments, such structure and/or linkages are related to certain biological activity of a polypeptide. In certain embodiments, refolding is accomplished using any of a number of procedures known in the art. Exemplary methods include, but are not limited to, exposing the solubilized polypeptide agent to a pH typically above 7 in the presence of a chaotropic agent. An exemplary chaotropic agent is guanidine. In certain embodiments, the refolding/oxidation solution also contains a reducing agent and the oxidized form of that reducing agent. In certain embodiments, the reducing agent and its oxidized form are present in a ratio that will generate a particular redox potential that allows disulfide shuffling to occur. In certain embodiments, such shuffling allows the formation of cysteine bridges. Exemplary redox couples include, but are not limited to, cysteine/cystamine, glutathione/dithiobisGSH, cupric chloride, dithiothreitol DTT/dithiane DTT, and 2-mercaptoethanol (bME)/dithio-bME. In certain embodiments, a co-solvent is used to increase the efficiency of refolding. Exemplary cosolvents include, but are not limited to, glycerol, polyethylene glycol of various molecular weights, and arginine.

In certain embodiments, one substantially purifies a polypeptide comprising one or more agonist anti-CD40 antibody components or the agonist anti-CD40 antibody itself. Certain protein purification techniques are known to those of skill in the art. In certain embodiments, protein purification involves crude fractionation of polypeptide fractionations from non-polypeptide fractions. In certain embodiments, polypeptides are purified using chromatographic and/or electrophoretic techniques. Exemplary purification methods include, but are not limited to, precipitation with ammonium sulphate; precipitation with PEG; immunoprecipitation; heat denaturation followed by centrifugation; chromatography, including, but not limited to, affinity chromatography (e.g., Protein-A-Sepharose), ion exchange chromatography, exclusion chromatography, and reverse phase chromatography; gel filtration; hydroxyapatite chromatography; isoelectric focusing; polyacrylamide gel electrophoresis; and combinations of such and other techniques. In certain embodiments, a polypeptide is purified by fast protein liquid chromatography or by high pressure liquid chromotography (HPLC). In certain embodiments, purification steps can be changed or certain steps can be omitted, and still result in a suitable method for the preparation of a substantially purified polypeptide.

In certain embodiments, one quantitates the degree of purification of a polypeptide preparation. Certain methods for quantifying the degree of purification are known to those of skill in the art. Certain exemplary methods include, but are not limited to, determining the specific binding activity of the preparation and assessing the amount of a polypeptide within a preparation by SDS/PAGE analysis. Certain exemplary methods for assessing the amount of purification of a polypeptide preparation comprise calculating the binding activity of a preparation and comparing it to the binding activity of an initial extract. In certain embodiments, the results of such a calculation are expressed as “fold purification.” The units used to represent the amount of binding activity depend upon the particular assay performed.

In certain embodiments, a polypeptide comprising one or more agonist anti-CD40 antibody components or the agonist anti-CD40 antibody itself is partially purified. In certain embodiments, partial purification can be accomplished by using fewer purification steps or by utilizing different forms of the same general purification scheme. For example, in certain embodiments, cation-exchange column chromatography performed utilizing an HPLC apparatus will generally result in a greater “fold purification” than the same technique utilizing a low-pressure chromatography system. In certain embodiments, methods resulting in a lower degree of purification can have advantages in total recovery of polypeptide, or in maintaining binding activity of a polypeptide.

In certain instances, the electrophoretic migration of a polypeptide can vary, sometimes significantly, with different conditions of SDS/PAGE. See, e.g., Capaldi et al., Biochem. Biophys. Res. Comm., 76: 425 (1977). It will be appreciated that under different electrophoresis conditions, the apparent molecular weights of purified or partially purified polypeptide can be different.

C. Nucleic Acid Molecule Encoding Agonist Anti-CD40 Antibodies

In an embodiment, the invention comprises a nucleic acid molecule encoding the isolated agonist anti-CD40 antibody as disclosed herein.

One of skill in the art will appreciate that the above discussion can be used for identifying, evaluating, and/creating agonist anti-CD40 antibodies and also for nucleic acid sequences that can encode for those antibodies. Thus, nucleic acid sequences encoding for those antibodies are contemplated. For example, an antibody can have at least 80, 80-85, 85-90, 90-95, 95-97, 97-99% or greater identity to at least one nucleic acid sequence described in SEQ ID NOs: 9 or 10 or at least one to six (and various combinations thereof) of the CDR(s) encoded by the nucleic acid sequences in SEQ ID NOs: 9 or 10.

In some embodiments, the antibody (or nucleic acid sequence encoding it) is contemplated within the invention if the nucleic acid sequence that encodes the particular antibody (or the nucleic acid sequence itself) can selectively hybridize to any of the nucleic acid sequences that encode the proteins SEQ ID NO: 7 and 8 under stringent conditions. In one embodiment, suitable moderately stringent conditions include prewashing in a solution of 5×SSC; 0.5% SDS, 1.0 mM EDTA (pH 8:0); hybridizing at 50° C., −65° C., 5×SSC, overnight or, in the event of cross-species homology, at 45° C. with 0.5×SSC; followed by washing twice at 65° C. for 20 minutes with each of 2×, 0.5×and 0.2×SSC containing 0.1% SDS. Such hybridizing DNA sequences are also within the scope of this invention, as are nucleotide sequences that, due to code degeneracy, encode an antibody polypeptide that is encoded by a hybridizing DNA sequence and the amino acid sequences that are encoded by these nucleic acid sequences. In some embodiments, variants of CDRs include nucleic acid sequences and the amino acid sequences encoded by those sequences, that hybridize to one or more of the CDRs within the sequences noted above.

The phrase “selectively hybridize” referred to in this context means to detectably and selectively bind. Polynucleotides, oligonucleotides and fragments thereof in accordance with the invention selectively hybridize to nucleic acid strands under hybridization and wash conditions that minimize appreciable amounts of detectable binding to nonspecific nucleic acids. High stringency conditions can be used to achieve selective hybridization conditions as known in the art and discussed herein. Generally, the nucleic acid sequence homology between the polynucleotides, oligonucleotides, and fragments of the invention and a nucleic acid sequence of interest will be at least 80%, and more typically with preferably increasing homologies of at least 85%, 90%, 95%, 99%, and 100%. Two amino acid sequences are homologous if there is a partial or complete identity between their sequences. For example, 85% homology means that 85% of the amino acids are identical when the two sequences are aligned for maximum matching. Gaps (in either of the two sequences being matched) are allowed in maximizing matching; gap lengths of 5 or less are preferred with 2 or less being more preferred. Alternatively and preferably, two protein sequences (or polypeptide sequences derived from them of at least 30 amino acids in length) are homologous, as this term is used herein, if they have an alignment score of at more than 5 (in standard deviation units) using the program ALIGN with the mutation data matrix and a gap penalty of 6 or greater. See Dayhoff, M. O., in Atlas of Protein Sequence and Structure, pp. 101-110 (Volume 5, National Biomedical Research Foundation (1972)) and Supplement 2 to this volume, pp. 1-10. The two sequences or parts thereof are more preferably homologous if their amino acids are greater than or equal to 50% identical when optimally aligned using the ALIGN program. The term “corresponds to” is used herein to mean that a polynucleotide sequence is homologous (i.e., is identical, not strictly evolutionarily related) to all or a portion of a reference polynucleotide sequence, or that a polypeptide sequence is identical to a reference polypeptide sequence. In contradistinction, the term “complementary to” is used herein to mean that the complementary sequence is homologous to all or a portion of a reference polynucleotide sequence. For illustration, the nucleotide sequence “TATAC” corresponds to a reference sequence “TATAC” and is complementary to a reference sequence “GTATA”.

In an embodiment, the invention comprises a vector comprising a nucleic acid molecule as described herein.

In an embodiment, the invention comprises a host cell comprising a nucleic acid molecule as described herein.

D. Compositions Comprising at Least One Agonist Anti-CD40 Antibody

In an embodiment, the invention comprises a composition comprising at least one agonist anti-CD40 antibody described herein. Preferably, the composition is a pharmaceutical composition. Accordingly, in a preferred for the invention comprises a pharmaceutical composition comprising at least one isolated agonist anti-CD40 antibody as described herein and a pharmaceutically acceptable excipient.

In an alternate embodiment, the invention provides for pharmaceutical compositions comprising an agonist anti-CD40 antibody together with a pharmaceutically acceptable diluent, carrier, solubilizer, emulsifier, preservative and/or adjuvant.

In certain embodiments, the invention provides for pharmaceutical compositions comprising an agonist anti-CD40 antibody and a therapeutically effective amount of at least one additional therapeutic agent, together with a pharmaceutically acceptable diluent, carrier, solubilizer, emulsifier, preservative and/or adjuvant.

Where the composition includes at least one additional therapeutic agent, that agent is preferably selected from the group consisting of a radioisotope, radionuclide, a toxin, or a therapeutic and a chemotherapeutic group. Accordingly, in a preferred form the invention comprises a pharmaceutical formulation comprising: an effective amount of at least one agonist anti-CD40 antibody disclosed herein together with at least a second immune enhancing agent. Agents include, but are not limited to, in vitro synthetically prepared chemical compositions, antibodies, antigen binding regions, and combinations and conjugates thereof. In certain embodiments, an agent can act as an agonist, antagonist, alllosteric modulator, or toxin. In certain embodiments, an agent can act to inhibit or stimulate its target, and thereby promote an immune response to a malignancy. Such immune enhancing agents include, without limitation, IL-2, TLR-7 agonists, or systemic cytotoxic chemotherapeutic agents.

In certain embodiments, acceptable formulation materials preferably are nontoxic to recipients at the dosages and concentrations employed. In some embodiments, the formulation material(s) are for s.c. and/or intratumoral administration. In certain embodiments, the pharmaceutical composition can contain formulation materials for modifying, maintaining or preserving, for example, the pH, osmolarity, viscosity, clarity, color, isotonicity, odor, sterility, stability, rate of dissolution or release, adsorption or penetration of the composition. In certain embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); colouring, flavouring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. (Remington's Pharmaceutical Sciences, 18th Edition, A. R. Gennaro, ed., Mack Publishing Company (1995). In some embodiments, the formulation comprises PBS; 20 mM NaOAC, pH 5.2, 50 mM NaCl; and/or 10 mM NAOAC, pH 5.2, 9% Sucrose.

In certain embodiments, an agonist anti-CD40 antibody and/or a therapeutic molecule is linked to a half-life extending vehicle known in the art. Such vehicles include, but are not limited to, polyethylene glycol, glycogen (e.g., glycosylation of the ABP), and dextran. Such vehicles are described, e.g., in U.S. application Ser. No. 09/428,082, now U.S. Pat. No. 6,660,843 and published PCT Application No. WO 99/25044, which are hereby incorporated by reference for any purpose.

In certain embodiments, the optimal pharmaceutical composition will be determined by one skilled in the art depending upon, for example, the intended route of administration, delivery format and desired dosage. See, for example, Remington's Pharmaceutical Sciences, supra. In certain embodiments, such compositions may influence the physical state, stability, rate of in vivo release and rate of in vivo clearance of the antibodies of the invention.

In certain embodiments, the primary vehicle or carrier in a pharmaceutical composition can be either aqueous or non-aqueous in nature. For example, in certain embodiments, a suitable vehicle or carrier can be water for injection, physiological saline solution or artificial cerebrospinal fluid, possibly supplemented with other materials common in compositions for parenteral administration. In some embodiments, the saline comprises isotonic phosphate-buffered saline. In certain embodiments, neutral buffered saline or saline mixed with serum albumin are further exemplary vehicles. In certain embodiments, pharmaceutical compositions comprise Tris buffer of about pH 7.0-8.5, or acetate buffer of about pH 4.0-5.5, which can further include sorbitol or a suitable substitute therefore. In certain embodiments, a composition comprising an agonist anti-CD40 antibody, with or without at least one additional therapeutic agents, can be prepared for storage by mixing the selected composition having the desired degree of purity with optional formulation agents (Remington's Pharmaceutical Sciences, supra) in the form of a lyophilized cake or an aqueous solution. Further, in certain embodiments, a composition comprising an agonist anti-CD40 antibody, with or without at least one additional therapeutic agent, can be formulated as a lyophilizate using appropriate excipients such as sucrose.

In certain embodiments, the pharmaceutical composition can be selected for parenteral delivery. The preparation of such pharmaceutically acceptable compositions is within the ability of one skilled in the art.

In certain embodiments, the formulation components are present in concentrations that are acceptable to the site of administration. In certain embodiments, buffers are used to maintain the composition at physiological pH or at a slightly lower pH, typically within a pH range of from about 5 to about 8.

In certain embodiments, when parenteral administration (preferably intra-tumorally or peri-tumorally) is contemplated, a therapeutic composition can be in the form of a pyrogen-free, parenterally acceptable aqueous solution comprising a desired agonist anti-CD40 antibody, with or without additional therapeutic agents, in a pharmaceutically acceptable vehicle. In certain embodiments, a vehicle for parenteral injection is sterile distilled water in which an agonist anti-CD40 antibody, with or without at least one additional therapeutic agent, is formulated as a sterile, isotonic solution, properly preserved. In certain embodiments, the preparation can involve the formulation of the desired molecule with an agent, such as injectable microspheres, bio-erodible particles, polymeric compounds (such as polylactic acid or polyglycolic acid), beads or liposomes, that can provide for the controlled or sustained release of the product which can then be delivered via a depot injection.

In certain embodiments, a pharmaceutical composition can involve an effective quantity of an agonist anti-CD40 antibody, with or without at least one additional therapeutic agent, in a mixture with non-toxic excipients which are suitable for the manufacture of tablets. In certain embodiments, by dissolving the tablets in sterile water, or another appropriate vehicle, solutions can be prepared in unit-dose form. In certain embodiments, suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc.

Additional pharmaceutical compositions will be evident to those skilled in the art, including formulations involving agonist anti-CD40 antibody, with or without at least one additional therapeutic agent(s), in sustained- or controlled-delivery formulations. In certain embodiments, techniques for formulating a variety of other sustained- or controlled-delivery means, such as liposome carriers, bio-erodible microparticles or porous beads and depot injections, are also known to those skilled in the art. See for example, PCT Application No. PCT/US93/00829 which describes the controlled release of porous polymeric microparticles for the delivery of pharmaceutical compositions. In certain embodiments, sustained-release preparations can include semipermeable polymer matrices in the form of shaped articles, e.g. films, or microcapsules. Sustained release matrices can include polyesters, hydrogels, polylactides (U.S. Pat. No. 3,773,919 and EP 058,481), copolymers of L-glutamic acid and gamma ethyl-L-glutamate (Sidman et al., Biopolymers, 22:547-556 (1983)), poly (2-hydroxyethyl-methacrylate) (Langer et al., J. Biomed. Mater. Res., 15:167-277 (1981) and Langer, Chem. Tech., 12:98-105 (1982)), ethylene vinyl acetate (Langer et aL, supra) or poly-D(−)-3-hydroxybutyric acid (EP 133,988). In certain embodiments, sustained release compositions can also include liposomes, which can be prepared by any of several methods known in the art. See, e.g., Eppstein et al., Proc. Natl. Acad. Sci. USA, 82:3688-3692 (1985); EP 036,676; EP 088,046 and EP 143,949.

The pharmaceutical composition to be used for in vivo administration typically is sterile. In certain embodiments, this can be accomplished by filtration through sterile filtration membranes. In certain embodiments, where the composition is lyophilized, sterilization using this method can be conducted either prior to or following lyophilization and reconstitution. In certain embodiments, the composition for parenteral administration can be stored in lyophilized form or in a solution. In certain embodiments, parenteral compositions generally are placed into a container having a sterile access port, for example, an intravenous solution bag or vial having a stopper pierceable by a hypodermic injection needle.

In certain embodiments, once the pharmaceutical composition has been formulated, it can be stored in sterile vials as a solution, suspension, gel, emulsion, solid, or as a dehydrated or lyophilized powder. In certain embodiments, such formulations can be stored either in a ready-to-use form or in a form (e.g., lyophilized) that is reconstituted prior to administration.

In certain embodiments, kits are provided for producing a single-dose administration unit. In certain embodiments, the kit can contain both a first container having a dried protein and a second container having an aqueous formulation. In certain embodiments, kits containing single and multi-chambered pre-filled syringes (e.g., liquid syringes and lyosyringes) are included.

In certain embodiments, the effective amount of a pharmaceutical composition comprising an agonist anti-CD40 antibody, with or without at least one additional therapeutic agent, to be employed therapeutically will depend, for example, upon the therapeutic context and objectives. One skilled in the art will appreciate that the appropriate dosage levels for treatment, according to certain embodiments, will thus vary depending, in part, upon the molecule delivered, the indication for which agonist anti-CD40 antibodies, with or without at least one additional therapeutic agent, are being used, the route of administration, and the size (body weight, body surface tumor size or organ size) and/or condition (the age and general health) of the subject. In certain embodiments, the clinician can titer the dosage and modify the route of administration to obtain the optimal therapeutic effect. In certain embodiments, a typical dosage can range from about 0.1 μg/kg to up to about 100 mg/kg or more, depending on the factors mentioned above. In certain embodiments, the dosage can range from 0.1 μg/kg up to about 100 mg/kg; or 1 μg/kg up to about 100 mg/kg; or 5 μg/kg up to about 100 mg/kg. For example, effective doses are preferably between 20 μg/kg and 200 μg/kg. Thus where a patient receives six doses, the corresponding total dose over 2 weeks is between 120 μg/kg and 1.2 mg/kg.

In certain embodiments, the frequency of dosing will take into account the pharmacokinetic parameters of the agonist anti-CD40 antibody and/or any additional therapeutic agents in the formulation used. In certain embodiments, a clinician will administer the composition until a dosage is reached that achieves the desired effect. In certain embodiments, the composition can therefore be administered as a single dose, or as two or more doses (which may or may not contain the same amount of the desired molecule) over time, or as a continuous infusion via an implantation device or catheter. Further refinement of the appropriate dosage is routinely made by those of ordinary skill in the art and is within the ambit of tasks routinely performed by them. In certain embodiments, appropriate dosages can be ascertained through use of appropriate dose-response data.

In certain embodiments, the route of administration of the pharmaceutical composition is in accord with known methods. Preferably through intratumoral or peritumoral injection however other routes of administration may be appropriate where the composition is formulated for site specific delivery. Such routes of administration might include intravenous, intraperitoneal, intramuscular, subcutaneously, intraarterial, intraportal, or intralesional routes; by sustained release systems or by implantation devices. In certain embodiments, the compositions can be administered by bolus injection or continuously by infusion, or by implantation device.

In certain embodiments, the composition can be administered locally via implantation of a membrane, sponge or another appropriate material onto which the desired molecule has been absorbed or encapsulated. In certain embodiments, where an implantation device is used, the device can be implanted into any suitable tissue or organ, and delivery of the desired molecule can be via diffusion, timed-release bolus, or continuous administration.

E. Methods for Treating or Preventing a Condition

In an embodiment, the invention comprises a method for treating or preventing a condition associated with malignancy in a patient, comprising administering to a patient in need thereof an effective amount of at least one isolated agonist anti-CD40 antibody disclosed herein.

In an embodiment, the invention comprises a method for increasing antigen presentation by APCs (including macrophages, DCs, and B cells), in a subject comprising administering an effective amount of at least one isolated agonist anti-CD40 antibody disclosed herein.

In an embodiment, the invention comprises a method of activating antigen presenting cells in a subject comprising administering an effective amount of at least one isolated agonist anti-CD40 antibody disclosed herein.

The activation of antigen presenting cells in a subject can be measured by assays known in the art. For example, antigen presentation by activated antigen presenting cells from the subject may be measured using a viral antigen recall assay in which PBMC from human donors previously exposed to Epstein-Barr virus (EBV) are challenged with EBV antigens in the presence of agonist anti-CD40 antibody.

In an embodiment, the invention comprises a method of activating dendritic cells in a subject comprising administering an effective amount of at least one isolated agonist anti-CD40 antibody disclosed herein.

The activation of dendritic cells in a subject can be measured by assays known in the art. For example, the stimulation of T-cells by activated dendritic cells from the subject may be measured using a mixed lymphocyte reaction.

In an embodiment, the invention comprises a method of enhancing the expression of MHC and/or immune costimulatory molecules in a subject comprising administering an effective amount of at least one isolated agonist anti-CD40 antibody disclosed herein. Preferably, the MHC and/or immune costimulatory molecules are selected from CD80, CD86, PD-L1, HLA-A,B,C, HLA-DR, and CD83.

The expression of MHC and/or immune costimulatory molecules in a subject can be measured by assays known in the art. For example, the expression of MHC and/or immune costimulatory molecules in cells from the subject may be measured using a fluorescence-activated cell sorting assay.

In an embodiment, the invention comprises a method of stimulating the production of pro-inflammatory cytokines, in a subject comprising administering an effective amount of at least agonist anti-CD40 antibody disclosed herein. Preferably, the pro-inflammatory cytokine is selected from IL-113, IL-6, IL-10, IL-12p40, IL-12p70, IL-23 and IFN-γ.

The expression of pro-inflammatory cytokines in a subject can be measured by assays known in the art. For example, the expression of pro-inflammatory cytokines in cells from the subject may be measured using an ELISA or a LEGENDplex assay.

In an embodiment, the invention comprises a method of inducing T cell activation, in a subject comprising administering an effective amount of at least one agonist anti-CD40 antibody disclosed herein.

In an embodiment, the invention comprises a method to mimic the signal of CD40L and substitute for the function of CD4+ lymphocytes, in a subject comprising administering an effective amount of at least one agonist anti-CD40 antibody disclosed herein.

In an embodiment, the invention comprises a method for overcoming T cell tolerance in tumor-bearing animals or evoking effective cytotoxic T cell responses or enhance the efficacy of anti-tumor vaccines, in a subject comprising administering an effective amount of at least one agonist anti-CD40 antibody disclosed herein.

In an embodiment, the invention comprises a method of promoting the secretion of autoantibodies directed against antigen expressed on tumour cells by B cells, in a subject comprising administering an effective amount of an agonist anti-CD40 antibody disclosed herein.

In an embodiment, the invention comprises a method of upregulating costimulatory markers and releasing IL-12 to activate CD8+ T cells and stimulating a specific cytotoxic T-cell response to cross-presented tumour antigens in a subject comprising administering an effective amount of an agonist anti-CD40 antibody disclosed herein.

The complex orchestration of events required for tumor eradication suggests that a combined therapeutic approach can also be beneficial in some circumstances. Agonist anti-CD40 antibodies can be administered together with additional arms for releasing antigen, promoting cytokine release, increasing immunosurveillance, and reducing the suppressive network to boost this effect.

In another embodiment, the invention comprises a method for treating or preventing a condition associated with malignancy in a patient, comprising administering to a patient in need thereof an effective amount of at least one isolated agonist anti-CD40 antibody disclosed herein and an effective amount of at least a second immune enhancing agent.

### Co-Administration of IL-2 and a CD40 Agonist

One therapeutic option is to alter the tumor microenvironment itself, encouraging the tumor to act as its own source of antigenic stimulation. This can be achieved by introducing IL-2 with anti-CD40 antibody into or near the tumor site. When directly co-injected the co-administration avoids the toxicity associated with systemic administration and successfully causes regression of larger tumors, as well as distal tumors, while preserving long-term protective memory. Co-administration of IL-2 and a CD40 agonist can lead to increased macrophage activity and T cell and B cell activation. A combination of IL-2 and a CD40 agonist can display remarkable benefit against various cancers with regression linked to a neutrophil and T cell co-dominant inflammatory response.

Accordingly, in an embodiment, the invention comprises a pharmaceutical composition comprising at least one agonist anti-CD40 antibody described herein and IL-2.

In an embodiment, the invention comprises a method for treating or preventing a condition associated with malignancy in a patient, comprising administering to a patient in need thereof an effective amount of each of, at least one agonist anti-CD40 antibody described herein and IL-2.

In an embodiment, the invention comprises a method for increasing antigen presentation by APCs (including macrophages, DCs, and B cells), in a subject comprising administering an effective amount of each of, at least one agonist anti-CD40 antibody described herein and IL-2.

In an embodiment, the invention comprises a method of activating antigen presenting cells in a subject comprising administering an effective amount of at least one isolated agonist anti-CD40 antibody disclosed herein and IL-2.

The activation of antigen presenting cells in a subject can be measured by assays known in the art. For example, antigen presentation by activated antigen presenting cells from the subject may be measured using a viral antigen recall assay in which PBMC from human donors previously exposed to Epstein-Barr virus (EBV) are challenged with EBV antigens in the presence of agonist anti-CD40 antibody.

In an embodiment, the invention comprises a method of activating dendritic cells in a subject comprising administering an effective amount of at least one isolated agonist anti-CD40 antibody disclosed herein and IL-2.

The activation of dendritic cells in a subject can be measured by assays known in the art. For example, the stimulation of T-cells by activated dendritic cells from the subject may be measured using a mixed lymphocyte reaction.

In an embodiment, the invention comprises a method of enhancing the expression of MHC and/or immune costimulatory molecules in a subject comprising administering an effective amount of each of, at least one agonist anti-CD40 antibody described herein and IL-2. Preferably, the MHC and/or immune costimulatory molecules are selected from CD80, CD86, PD-L1, HLA-A, B, C, HLA-DR, and CD83.

The expression of MHC and/or immune costimulatory molecules in a subject can be measured by assays known in the art. For example, the expression of MHC and/or immune costimulatory molecules in cells from the subject may be measured using a fluorescence-activated cell sorting assay.

In an embodiment, the invention comprises a method of stimulates the production of pro-inflammatory cytokines, in a subject comprising administering an effective amount of each of, at least one agonist anti-CD40 antibody described herein and IL-2. Preferably, the pro-inflammatory cytokine is selected from IL-113, IL-6, IL-10, IL-12p40, IL-12p70, IL-23 and IFN-γ.

The expression of pro-inflammatory cytokines in a subject can be measured by assays known in the art. For example, the expression of pro-inflammatory cytokines in cells from the subject may be measured using an ELISA or a LEGENDplex assay.

In an embodiment, the invention comprises a method of inducing T cell activation, in a subject comprising administering an effective amount of each of, at least one agonist anti-CD40 antibody described herein and IL-2.

In an embodiment, the invention comprises a method to mimic the signal of CD40L and substitute for the function of CD4+ lymphocytes, in a subject comprising administering an effective amount of each of, at least one agonist anti-CD40 antibody described herein and IL-2.

In an embodiment, the invention comprises a method for overcoming T cell tolerance in tumor-bearing animals or evoking effective cytotoxic T cell responses or enhance the efficacy of anti-tumor vaccines, in a subject comprising administering an effective amount of each of, at least one agonist anti-CD40 antibody described herein and IL-2.

In an embodiment, the invention comprises a method of promoting the secretion of autoantibodies directed against antigen expressed on tumour cells by B cells, in a subject comprising administering an effective amount of an agonist anti-CD40 antibody disclosed herein and IL-2.

In an embodiment, the invention comprises a method of upregulating costimulatory markers and releasing IL-12 to activate CD8+ T cells and stimulating a specific cytotoxic T-cell response to cross-presented tumour antigens in a subject comprising administering an effective amount of an agonist anti-CD40 antibody disclosed herein and IL-2.

The most effective method for treating or preventing a condition associated with malignancy in a patient may require a combined therapeutic approach in which therapeutic interventions are carried out in sequence over time.

Accordingly, in an embodiment the invention comprises a method for treating or preventing a condition associated with malignancy in a patient, comprising administering to a patient in need thereof an effective amount of at least one isolated agonist anti-CD40 antibody disclosed herein in sequence over time with an additional therapeutic intervention. In some embodiments, said additional therapeutic intervention is selected from the group consisting of surgery, radiotherapy, chemotherapy, thermotherapy and immunotherapy.

One therapeutic option is to alter cells isolated from a patient and then transfer these cells back into the patient. This can be achieved by treating cells isolated from a patient with anti CD40 antibody. Cells treated with anti CD40 antibody may be treated with additional agents. In some embodiments, said agents are selected from the group consisting of tumor-specific peptides, tumor cell lysates, cytokines, agonists and mitogens.

Accordingly, in an embodiment the invention comprises a method for treating or preventing a condition associated with malignancy in a patient, comprising administering to a patient in need thereof cells treated with an effective amount of at least one isolated agonist anti-CD40 antibody disclosed herein. In some embodiments, said cells have been isolated from the patient and are selected from the group consisting of DCs, macrophages, B cells, myeloid cells, lymphoid cells and haematopoietic stem cells.

Further features of the present invention are more fully described in the following Examples. It is to be understood, however, that this detailed description is included solely for the purposes of exemplifying the present invention and should not be understood in any way as a restriction on the broad description of the invention as set out above.

### EXAMPLES

### Example 1—Production of SVX-3001 Antibodies

### Sequence Data

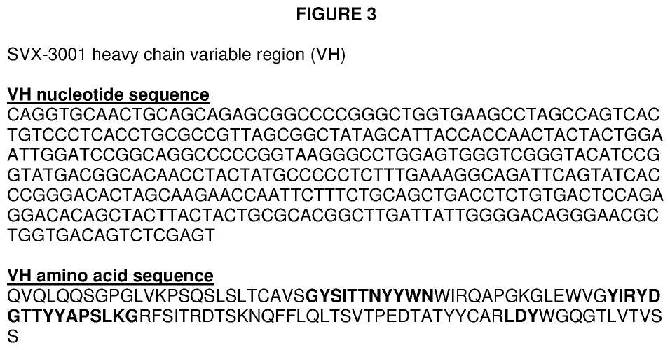

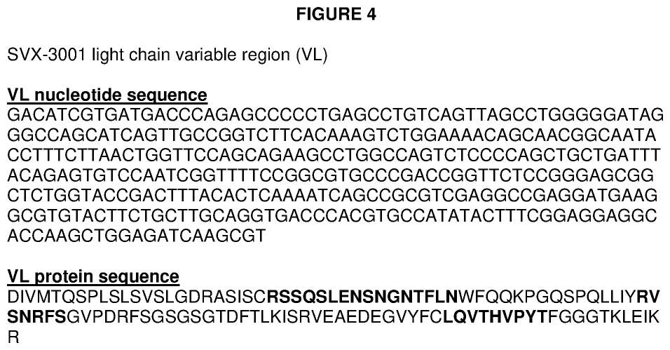

Nucleotide sequence for the heavy chain of a humanised agonist anti-human CD40 antibody (SEQ ID NO 13, FIG. 1) and the light chain of a humanised agonist anti-human CD40 antibody (SEQ ID NO 14, FIG. 2) were obtained from North Coast Biologics. In FIGS. 1 and 2, HindIII and XbaI sites are shown in bold and coding sequence for the heavy and light chains is underlined. The humanised agonist anti-human CD40 antibody was named SVX-3001. The coding sequence and amino acid sequence of the heavy chain variable region of SVX-3001 (SEQ ID NO 9 and SEQ ID NO 7, FIG. 3) and light chain variable region of SVX-3001 (SEQ ID NO 10, and SEQ ID NO 8, FIG. 4) were identified along with the amino acid sequences of the complementarity-determining regions; CDR1, CDR2 and CDR3. In FIGS. 3 and 4, the amino acid sequences of the complementarity-determining regions (CDR1, CDR2 and CDR3) are shown in bold.

### Gene Synthesis and Cloning

DNA sequences encoding the heavy chain of SVX-3001 (SEQ ID NO 15, FIG. 5) and the light chain of SVX-3001 (SEQ ID NO 17, FIG. 6) with HindII and XbaI restriction enzyme sites for cloning were synthesised by GenScript HK and cloned into vector pcDNA3.1(+) to generate plasmids pcDNA3.1(+)_Selvax01HC (FIG. 7) and pcDNA3.1(+)_Selvax01LC (FIG. 8) respectively. Genscript HK supplied 100 μg of each plasmid, transfection grade (90% supercoiled, 0.01 EU/ug endotoxin) in TE buffer. In FIGS. 5 and 6, HindIII and XbaI sites are shown in bold and the coding sequence is underlined. Amino acid sequence of the heavy/light chain variable region is shown in plain text, the signal peptide sequence is shown in bold, and the human IgG1 heavy chain constant region/human kappa light chain constant region is underlined.

### Production of SVX-3001 Antibody-Containing Supernatant

One day before transfection HEK 293T cells were seeded into 6-well tissue culture plates (Corning-Falcon) at a density of 4×10⁵ cells/well in in DMEM (Gibco) with 10% FCS (Hyclone). Media was removed three hours before transfection and replaced with 1.5 ml/well DMEM+1% FCS. Transfection mix was made by adding 1.5 μg of pcDNA3.1(+)_Selvax01HC DNA and 1.5 μg of pcDNA3.1(+)_Selvax01LC DNA to a sterile tube with 300 μl of DMEM (no additives) before adding 6 μl of polyethylenimine (PEI), 25 kD, linear (Polysciences) at 1 μg/ml and mixing by vortex. Transfection mix was incubated for 10 minutes at room temperature before adding to cells. Cells were incubated at 37° C. for 3 hours before adding 1.5 ml/well DMEM+10% FCS. Cells were then returned to culture at 37° C. with 5% CO₂.

SVX-3001 antibody-containing supernatant from transiently transfected cells was collected at 18 hours following transfection and 2 days following transfection and replaced with DMEM+10% FCS. Culture supernatant was collected for a final time at 7 days following transfection. All culture supernatants were centrifuged to remove cellular debris and stored at 4° C.

### Production of Purified SVX-3001 Antibody

One day before transfection HEK 293T cells were seeded into 6-well tissue culture plates (Corning-Falcon) at a density of 4×10⁵ cells/well in in DMEM (Gibco) with 10% ultra-low IgG FCS (Gibco). Media was removed three hours before transfection and replaced with 1.5 ml/well DMEM+1% ultra-low IgG FCS. Transfection mix was made by adding 1.5 μg of pcDNA3.1(+)_Selvax01HC and 1.5 μg of pcDNA3.1(+)_Selvax01LC to a sterile tube with 300 μl of DMEM (no additives) before adding 6 μl of polyethylenimine (PEI), 25 kD, linear (Polysciences) at 1 μg/ml and mixing by vortex. Transfection mix was incubated for 10 minutes at room temperature before adding to cells. Cells were incubated at 37° C. for 3 hours before adding 1.5 ml/well DMEM+10% ultra-low IgG FCS. Cells were then returned to culture at 37° C. with 5% CO₂.

SVX-3001 antibody-containing supernatant from transiently transfected cells was collected at 2 days following transfection and 5 days following transfection and replaced with DMEM+10% ultra-low IgG FCS. Culture supernatant was collected for a final time at 7 days following transfection. Culture supernatants from the different time points were pooled, filtered through a 0.22 μm filter and stored at 4° C. SVX-3001 antibody was purified from pooled supernatant using Protein G at the Harry Perkins Institute of Medical Research Monoclonal Antibody Facility. Purified SVX-3001 antibody was supplied at 1 mg/ml in PBS.

### Example 2—ELISA to Detect Human IgG with Specificity for Human CD40

ELISA was carried out using an IgG (Total) Human ELISA Kit with Plates (Invitrogen). Wells of an ELISA plate (Corning-Costar) were coated with 100 μl/well purified anti-human IgG monoclonal capture antibody at the recommended concentration or recombinant human CD40 extracellular domain with C-terminal 6His tag (Novoprotein) at 1 μg/ml. Plate was incubated overnight at 4° C. Wells were washed twice with 400 μl/well Wash Buffer before adding 250 μl/well Blocking Buffer. Plate was incubated for 2 hours at room temperature. Wells were washed twice with 400 μl/well Wash Buffer before adding 100 μl/well serial 1/2 dilutions of recombinant human IgG standard from 100 ng/ml to 1.56 ng/ml, serial 1/2 dilutions of control anti-CD40 antibody Lob 7/4 IgG1 (University of Southampton) from approximately 100 ng/ml to 1.56 ng/ml, and serial 1/10 dilutions of supernatants from HEK 293T cells transiently transfected with plasmids pcDNA3.1(+)_Selvax01HC and pcDNA3.1(+)_Selvax01LC from 1/10 to 1/10,000. Plate was incubated for 2 hours at room temperature on a microplate shaker set to 400 rpm. Wells were washed four times with 400 μl/well Wash Buffer before adding 100 μl/well Substrate Solution containing tetramethylbenzidine (TMB). Plate was incubated for 15 minutes at room temperature. Added 100 μl of Stop Solution (1 M H3PO4) to each well. Tests included diluent only control (no antibody), recombinant human IgG standard (25 ng/ml), SVX-3001 antibody (estimated at 27.3 ng/ml) and Lob 7/4 IgG1 antibody (estimated at 42.2 ng/ml). Captured antibody was detected using an HRP anti-human IgG antibody. Absorbance at 450 nm was measured using an EnSpire Multimode Plate Reader (Perkin Elmer).

Results: Recombinant human IgG standard could be detected in wells coated with purified anti-human IgG monoclonal capture antibody but not in wells coated with recombinant CD40 protein (FIG. 9). Anti-CD40 antibodies SVX-3001 and Lob 7/4 IgG1 could be detected in wells coated with purified anti-human IgG monoclonal capture antibody and in wells coated with recombinant CD40 protein (FIG. 9).

Conclusions: Supernatants from HEK 293T cells transiently transfected with plasmids pcDNA3.1(+)_Selvax01HC and pcDNA3.1(+)_Selvax01LC were confirmed to contain human IgG protein with specificity for human CD40 protein (i.e. antibody SVX-3001).

### Example 3—FACS Analysis to Detect Binding of Antibody to Antigen on Cells

FACS analysis was conducted to determine whether SVX-3001 binds to CD40 expressed on the surface of human peripheral blood mononuclear cells (PBMC). PMBC were stained with an anti-human CD19 antibody labelled with PE. SVX-3001 antibody bound to the cell surface was detected using an anti-human IgG antibody labelled with BV421. Lymphocytes were gated based on FSC-A and SSC-A signals.

Method: Human buffy coat samples were obtained from the Australian Red Cross Blood Service (Ethics approval: RDHS-243-15, Human Research Ethics Office, Curtin University). Peripheral blood mononuclear cells (PBMC) were isolated from human buffy coat samples by density centrifugation using Ficoll-Paque PLUS (GE Healthcare Life Sciences).

FACS staining was carried out in 96-well U-bottom plates (Corning-Falcon). All incubations were carried out on ice in the dark. The FACS buffer used for wash steps and to dilute antibodies consisted of PBS with 1% BSA (Sigma), 1% FCS (Hyclone) and 0.01% w/v sodium azide (Sigma). Staining reagents were SVX-3001 antibody-containing supernatants, human IgG1 isotype control (BioLegend), BV421 anti-mouse Ig (BD Biosciences) and PE anti-human CD19 (BioLegend).

FACS staining was carried out on 10⁶ human PBMC per well. Cells were pelleted and resuspended in 20 μl of SVX-3001 antibody-containing supernatant and incubated for 30 minutes. Cells were washed twice with FACS buffer and resuspended in 20 μl of BV421 anti-mouse Ig diluted in FACS buffer and incubated for 30 minutes. Cells were washed twice with FACS buffer and resuspended in 20 μl of PE anti-human CD19 diluted in FACS buffer and incubated for 30 minutes. Cells were washed once with FACS buffer and once with PBS and resuspended in 100 μl of 1% formaldehyde in PBS and incubated for 20 minutes. Cells were washed twice with FACS buffer and resuspended in 200 μl of FACS buffer for analysis.

FACS staining controls included an unstained control, PE anti-human CD19 and BV421 mouse anti-human Ig single stain controls and human IgG1 isotype control. Cell staining was analysed using a FACS Canto II flow cytometer (BD). PMT voltages and gates for analysis were set using the FACS staining controls.

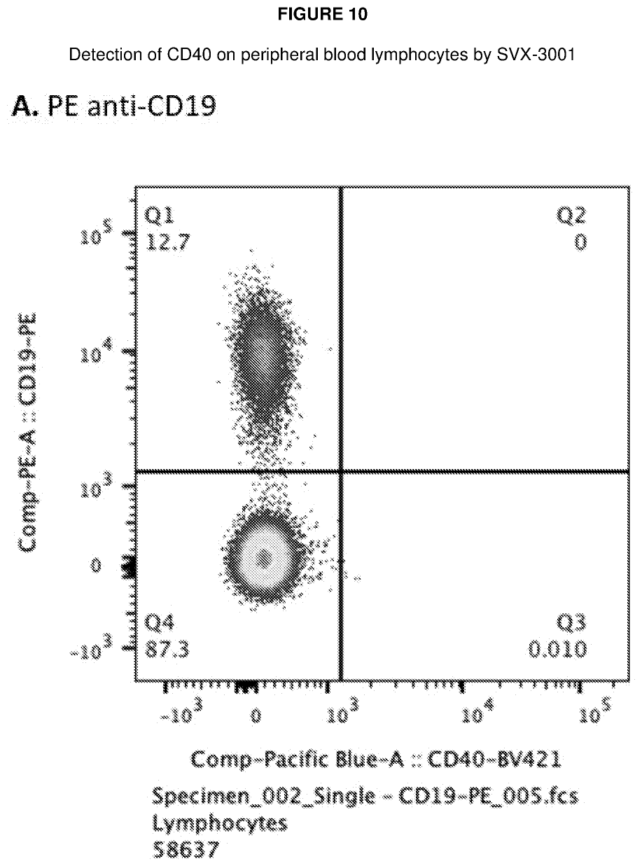

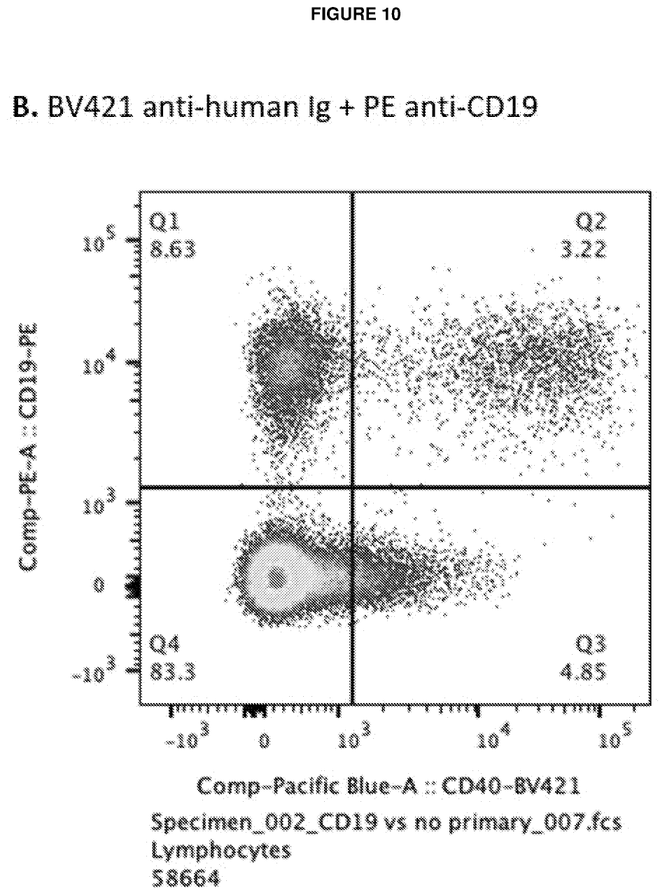

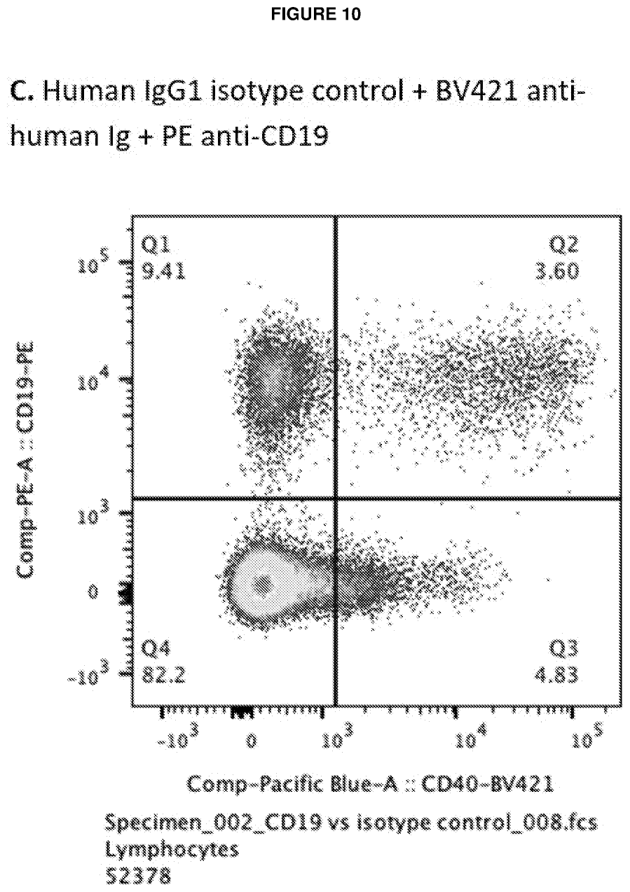

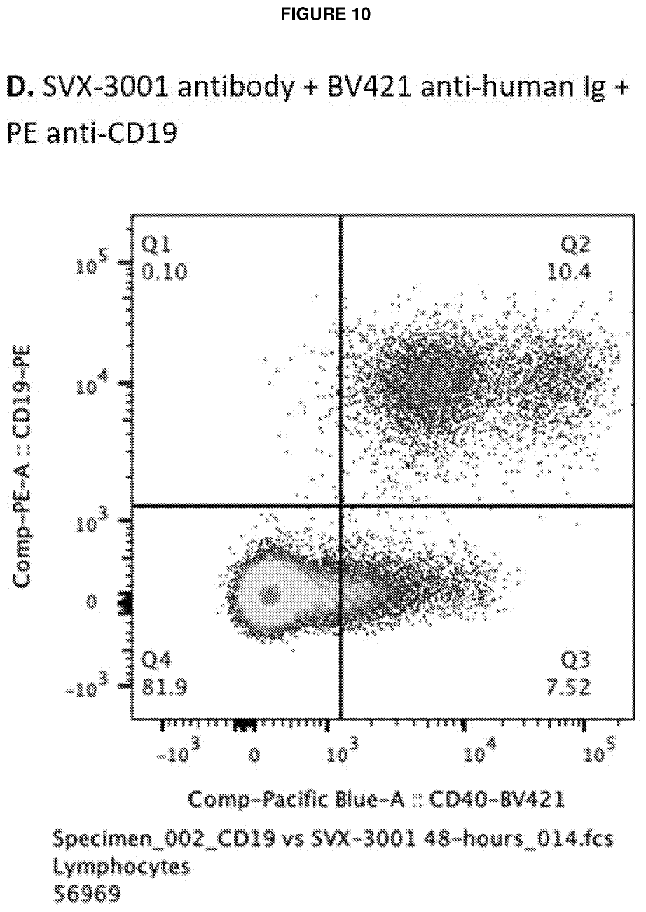

Results: SVX-3001 antibody-containing supernatant bound to CD40 on the surface of CD19⁺ peripheral blood lymphocytes (FIG. 10). FIG. 10 (A) shows PE anti-CD19 control showing PE fluorescence due to the staining of cells expressing CD19 on the cell surface. FIG. 10 (B) shows BV421 anti-human IgG control showing background BV421 fluorescence due to binding of the anti-human IgG antibody to a subset of CD19⁺ cells expressing IgG on the cell surface. FIG. 10 (C) shows staining with human IgG1 isotype control. FIG. 10 (D) shows test staining with SVX-3001 antibody-containing supernatant showing BV421 fluorescence due to the presence of SVX-3001 antibody bound to CD40 on the surface of B cells.

Conclusions: SVX-3001 antibody has specificity for native CD40 protein expressed on the surface of human immune cells.

### Example 4—CFSE Assay to Detect Cell Division in Response to Stimulation

The ability of SVX-3001 to stimulate cell division rates of PMBC was measured using CFSE. CFSE-labelled human PBMC were cultured with SVX-3001 antibody or human IgG1 isotype control antibody at 1 μg/ml, 0.1 μg/ml, 0.01 μg/ml and 0.001 μg/ml for 7 days and then stained with Zombie Aqua and PE anti-CD19.

Method: Human buffy coat samples were obtained from the Australian Red Cross Blood Service (Ethics approval: RDHS-243-15, Human Research Ethics Office, Curtin University). Peripheral blood mononuclear cells (PBMC) were isolated from human buffy coat samples by density centrifugation using Ficoll-Paque PLUS (GE Healthcare Life Sciences).

Human PBMC were suspended at 2×10⁷ cells/ml in PBS and stained with CFSE (Life Technologies Australia) using 25 μl of 100 μM CFSE for every 1 ml of cells. Cells were mixed with CFSE by gentle inversion for 10 minutes before adding at least 4 volumes of RPMI (Gibco)+10% FCS (Hyclone). Cells were washed twice with RPMI+10% FCS before resuspending for culture in RPMI+10% FCS.

Cells were cultured in 96-well plates (Nunc) at 5×10⁵ cells/well in a final volume of 200 μl/well of RPMI+10% FCS. Cells were stimulated with SVX-3001 antibody-containing supernatant or human IgG1 isotype control antibody (BioLegend) at antibody concentrations of 1 mg/ml, 0.1 mg/ml, 0.01 mg/ml and 0.01 mg/ml. Cells were cultured for 7 days at 37° C. with 5% CO₂ and then harvested for FACS staining.

FACS staining was carried out in 96-well U-bottom plates (Corning-Falcon). All incubations were carried out on ice in the dark. The FACS buffer used for wash steps and to dilute antibodies consisted of PBS with 1% BSA (Sigma), 1% FCS (Hyclone) and 0.01% w/v sodium azide (Sigma). Staining reagents were Zombie Aqua (BioLegend) and PE anti-human CD19 (BioLegend).

Cells for FACS staining were pelleted and resuspended in 20 μl of PE anti-human CD19 diluted in FACS buffer and incubated for 30 minutes. Cells were washed twice with PBS and resuspended in 100 μl of Zombie Aqua diluted in PBS and incubated for 15 minutes. Cells were washed once with FACS buffer and once with PBS and resuspended in 100 μl of 1% formaldehyde in PBS and incubated for 20 minutes. Cells were washed twice with FACS buffer and resuspended in 200 μl of FACS buffer for analysis.

FACS staining controls included an unstained control, single stain controls, and FMO controls. Cell staining was analysed using a FACS Canto II flow cytometer (BD). PMT voltages and gates for analysis were set using the FACS staining controls.

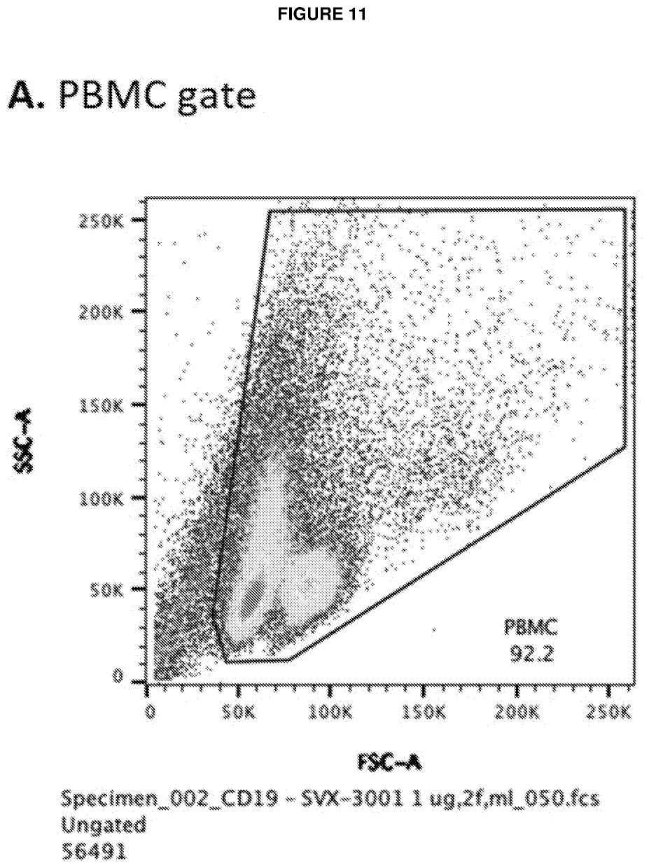

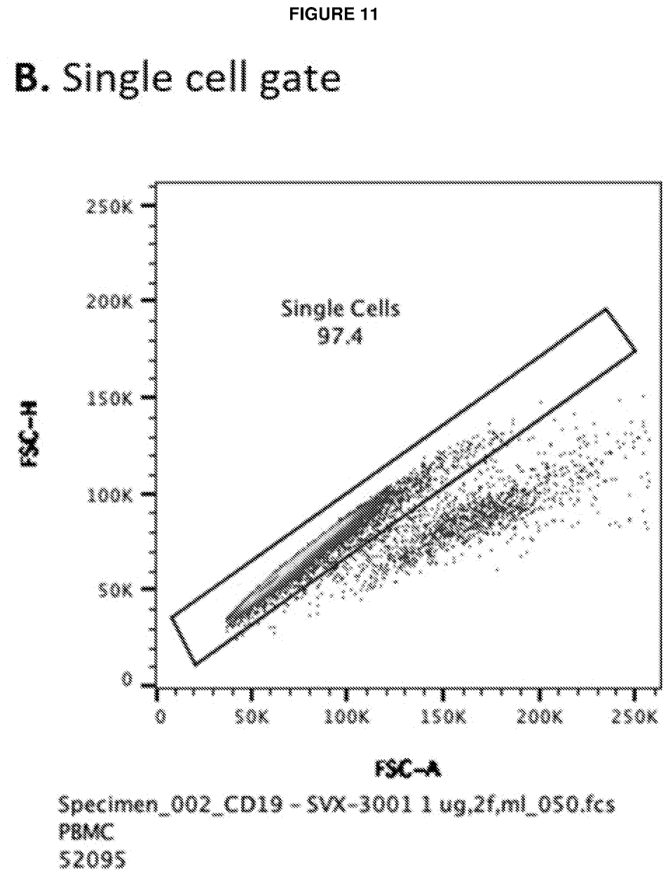

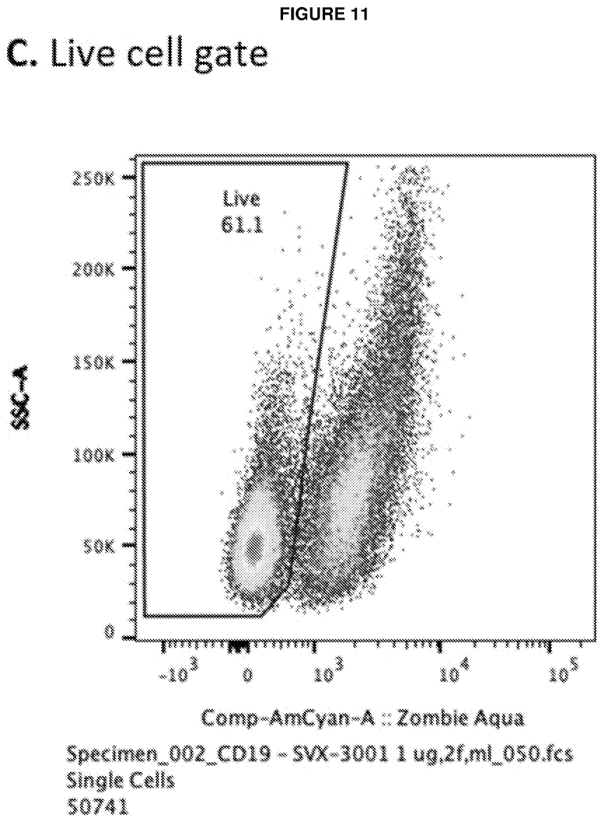

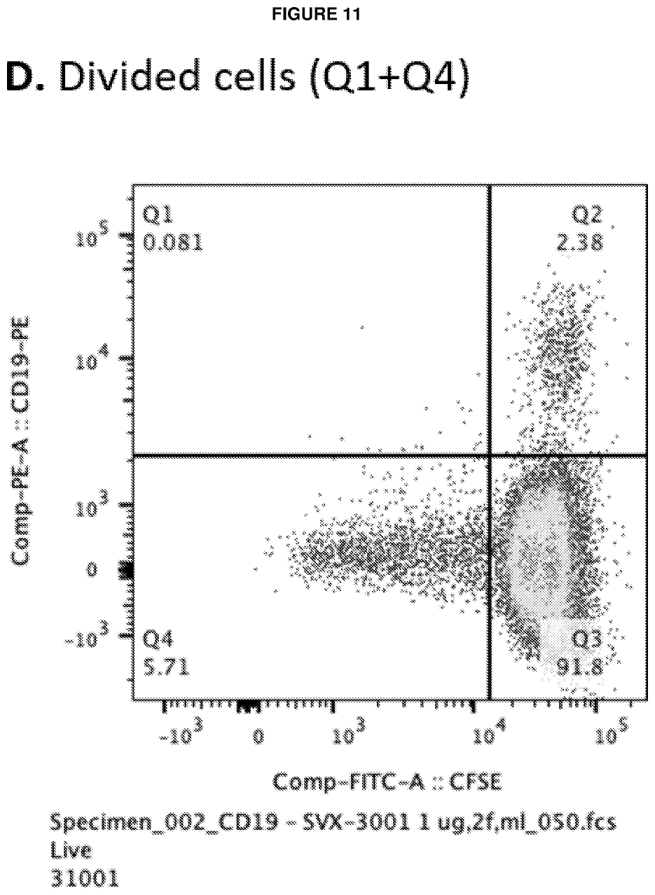

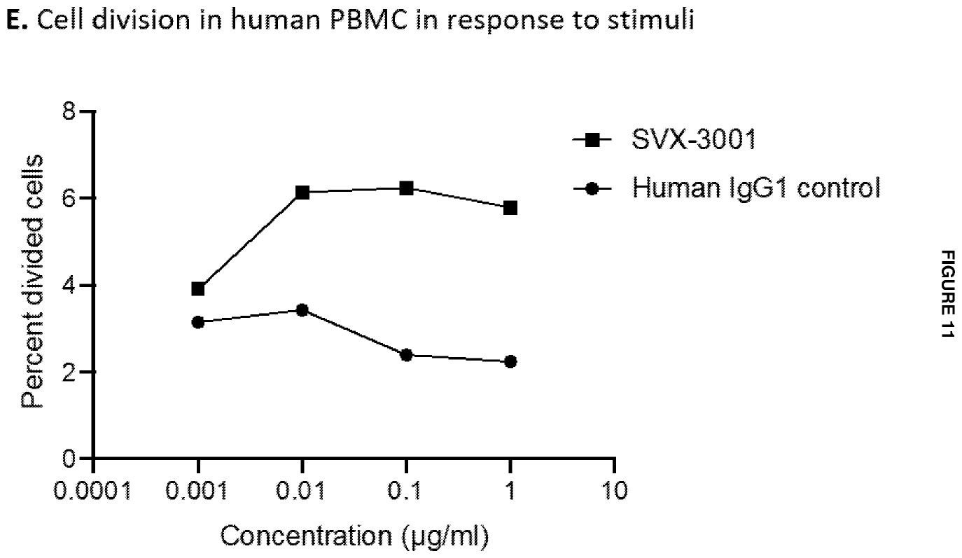

Results: SVX-3001 antibody stimulated cell division in human PBMC above the background level of cell division seen in human PBMC stimulated with a human IgG1 isotype control antibody at concentrations above 0.01 mg/ml (FIG. 11). FIG. 11 (A) shows PBMC gate on FSC-A versus SSC-A was used to exclude debris. FIG. 11 (B) shows single cell gate on FSC-A versus FSC-H was used to exclude cell clumps. FIG. 11 (C) shows live cell gate on Zombie Aqua versus SSC-A was used to exclude dead cells. FIG. 11 (D) shows divided cells were identified on the basis of a reduction in CFSE fluorescence on CFSE versus PE-anti-CD19. FIG. 11 (E) shows a graph of the percent divided cells in PBMC cultures stimulated with different concentrations of human IgG1 antibodies.

Conclusions: SVX-3001 antibody has stimulatory (agonist) activity that results in increased cell division human immune cells.

### Example 5—LEGENDplex Assay to Detect Cytokine Response to Stimulation of PMBCs with SVX-3001

A LEGENDplex assay was conducted in order to determine the level of cytokine production in response to stimulation of PMBCs with SVX-3001.

Method: Human blood samples were obtained from healthy volunteers (Ethics approval: HRE2017-0767, Human Research Ethics Office, Curtin University). Peripheral blood mononuclear cells (PBMC) were isolated from human blood samples by density centrifugation using Ficoll-Paque PLUS (GE Healthcare Life Sciences) and stored at −80° C. in FCS (Hyclone) with 10% DMSO (Sigma). Human PBMC were thawed and cultured in RPMI (Gibco)+10% FCS overnight at 37° C. with 5% FCS to allow the cells to recover from freezing. Cells were labelled using a Cell Trace Violet Proliferation Kit (Life Technologies Australia).

Cells were cultured in 96-well plates (Nunc) at 5×10⁵ cells/well in a final volume of 200 μl/well of RPMI+10% FCS. Cells were cultured with no stimulation, 1 μg/ml purified SVX-3001 antibody, 10 ng/ml recombinant human IL-2 (Peprotech) or 1 μg/ml purified SVX-3001 antibody and 10 ng/ml recombinant human IL-2. Cells were cultured for 3 days at 37° C. with 5% CO₂ and then supernatants were harvested. Supernatants were assayed for soluble analytes using LEGENDplex bead-based immunoassays (BioLegend). LEGENDplex data was acquired using a LSRFortessa Cell Analyser (BD).

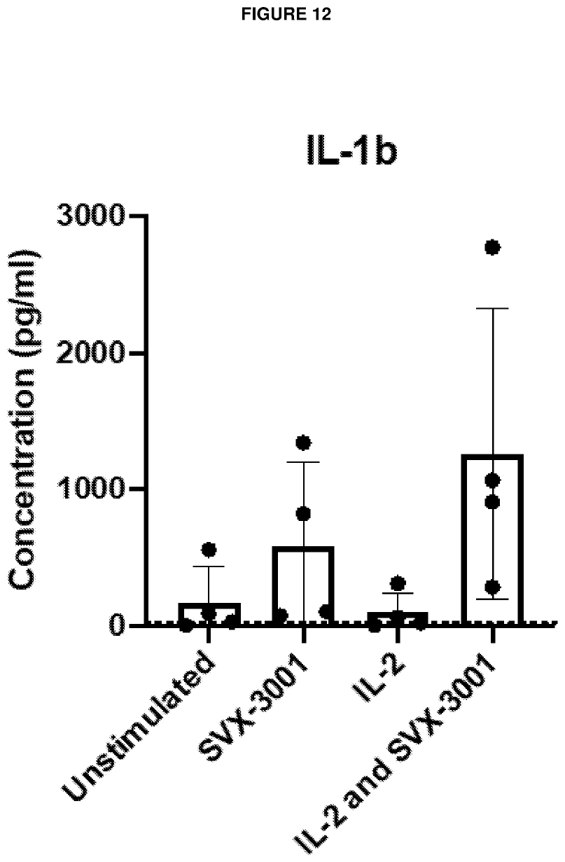

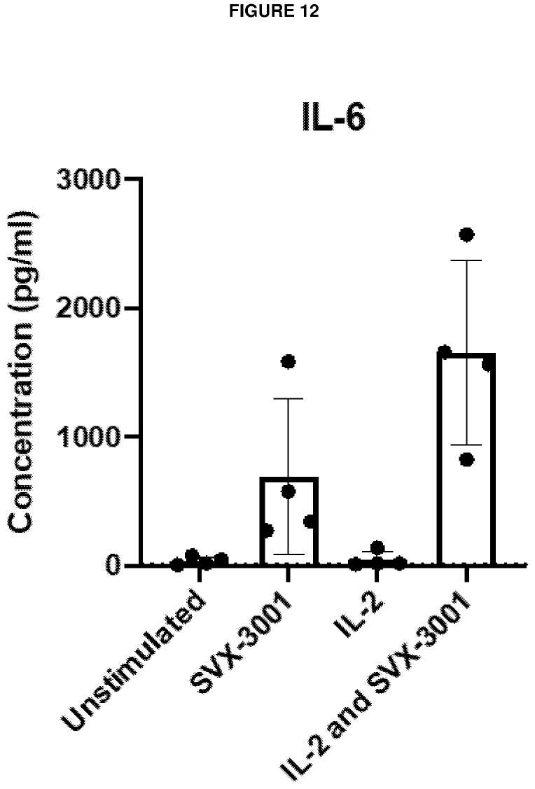

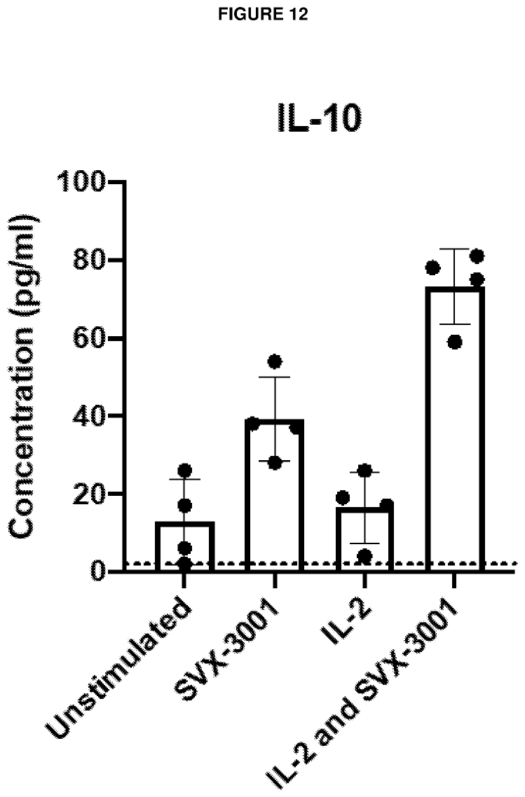

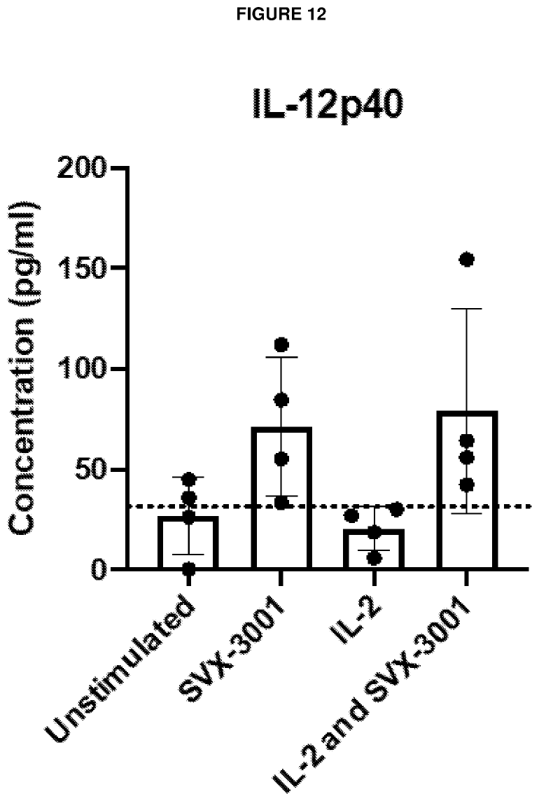

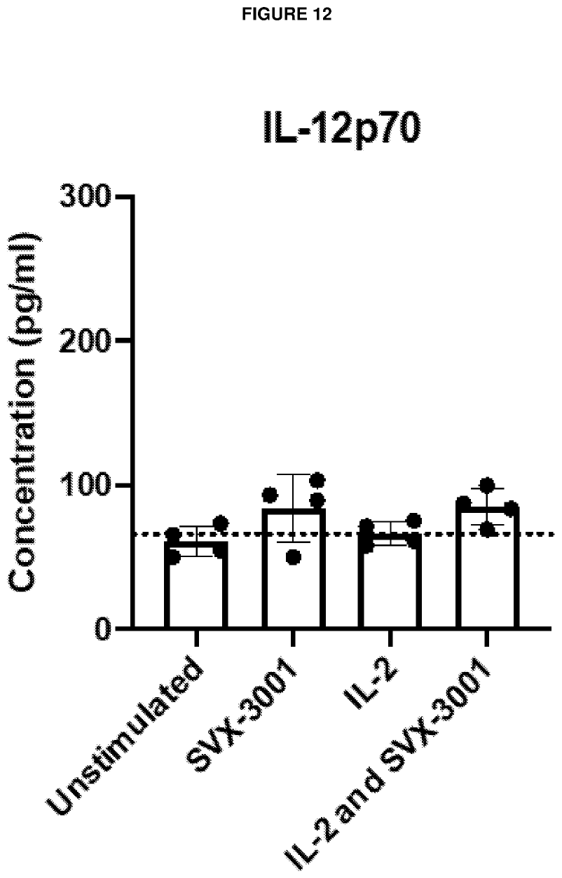

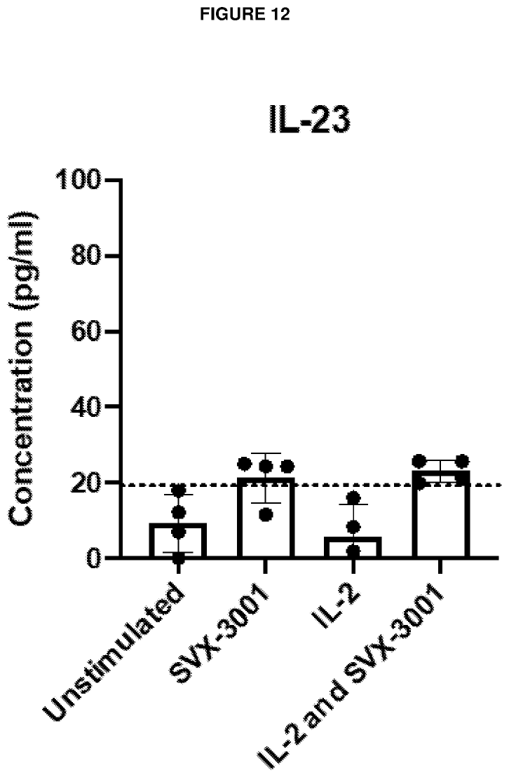

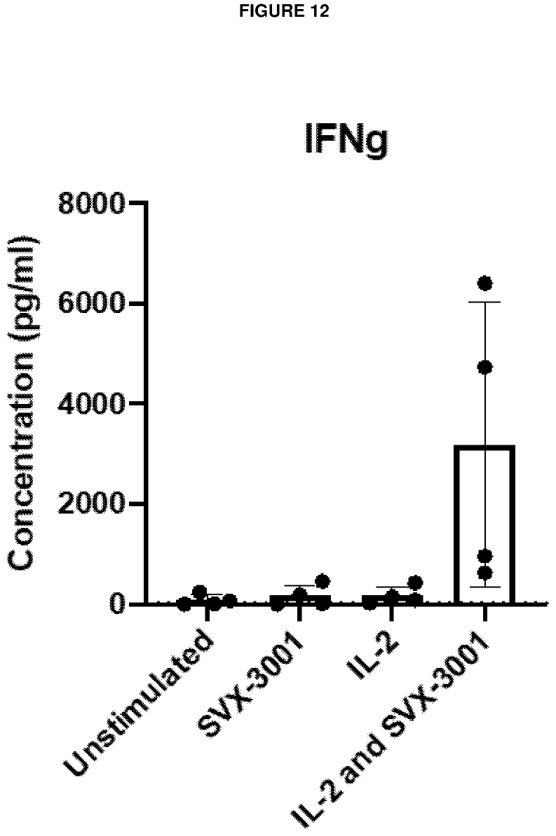

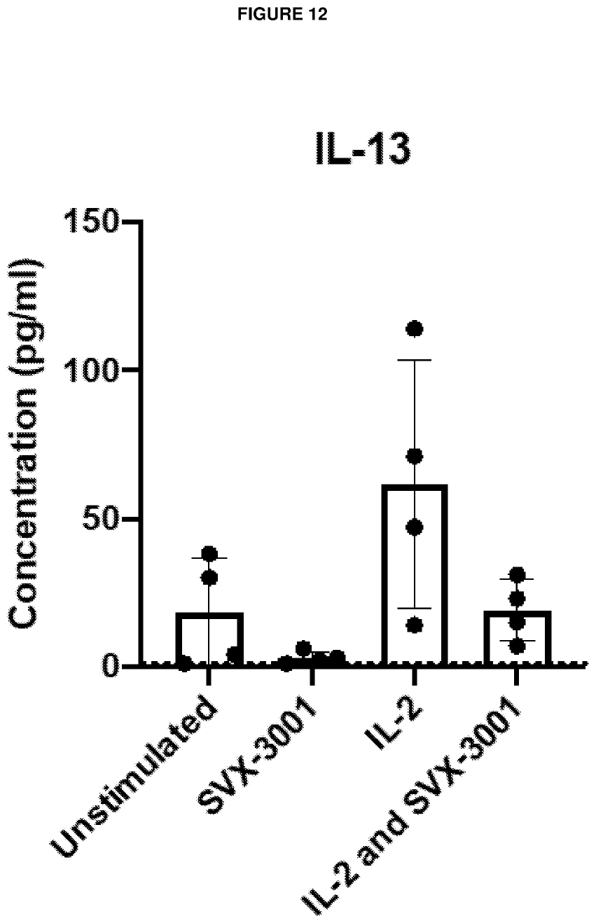

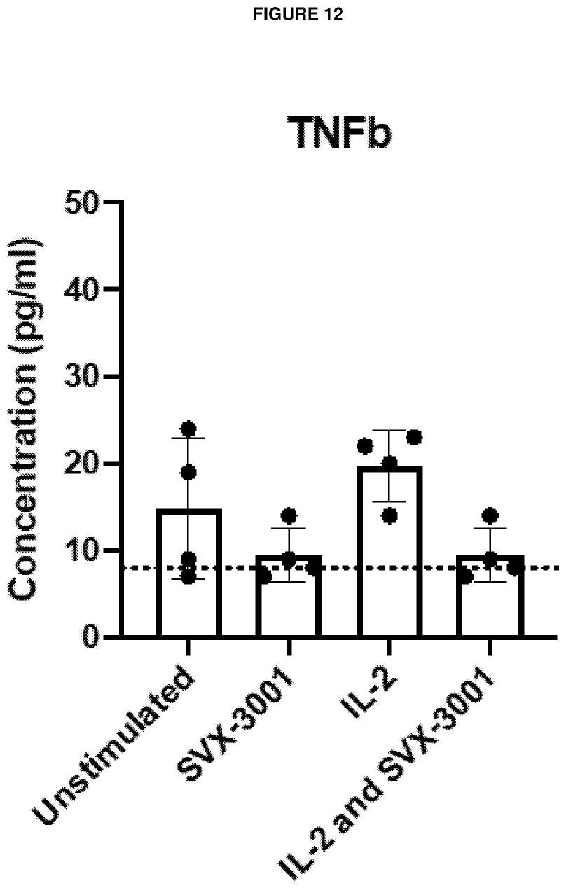

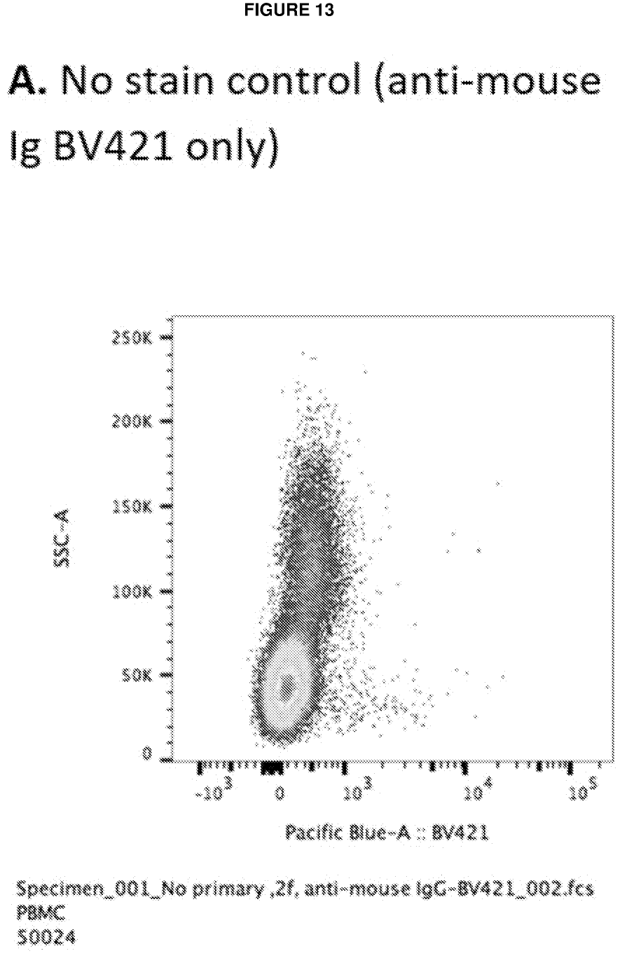

Results: Stimulation of human PBMC with purified SVX-3001 antibody resulted in an increase of IL-1 beta, IL-6, IL-10, IL-12p40, IL-12p70 and IL-23 and a decrease in IL-13 and TNF beta in the medium after 3 days culture (FIG. 12). Stimulation of human PBMC with the combination of SVX-3001 and IL-2, but not SVX-3001 or IL-2 alone, resulted in the release of IFN gamma into the medium after 3 days culture (FIG. 12). The cytokine concentration measured in the culture medium control (RPMI+10% FCS+10 ng/ml IL-2) is indicated in FIG. 12 as a dotted line.

Conclusions: SVX-3001 antibody has stimulatory (agonist) activity that results in the differential release of cytokines from human immune cells.

### Example 6—Antibody Competition Studies by FACS

A FACS assay was conducted to determine the ability of SVX-3001 to block the binding of antibodies B-B20 and LOB7/6 to CD40. Human PBMC were stained with mouse anti-human CD40 antibodies B-B20 and LOB7/6 in the presence or absence of SVX-3001 antibody at 1 μg/ml. Binding of the mouse anti-human CD40 antibodies to human PBMC was detected using an anti-mouse Ig antibody labelled with BV421.

Method: Human buffy coat samples were obtained from the Australian Red Cross Blood Service (Ethics approval: RDHS-243-15, Human Research Ethics Office, Curtin University). Peripheral blood mononuclear cells (PBMC) were isolated from human buffy coat samples by density centrifugation using Ficoll-Paque PLUS (GE Healthcare Life Sciences).

FACS staining was carried out in 96-well U-bottom plates (Corning-Falcon). All incubations were carried out on ice in the dark. The FACS buffer used for wash steps and to dilute antibodies consisted of PBS with 1% BSA (Sigma), 1% FCS (Hyclone) and 0.01% w/v sodium azide (Sigma). Blocking reagent was SVX-3001 antibody-containing supernatant. Staining reagents were anti-CD40 antibody LOB7/6 (LSBio), anti-CD40 antibody B-B20 (Abcam) and BV421 anti-mouse Ig (BD Biosciences).

FACS staining was carried out on 5×10⁵ human PBMC per well. Cells were pelleted and resuspended in 200 μl of SVX-3001 antibody-containing supernatant at 1 μg/ml, 0.1 μg/ml, 0.01 μg/ml or 0.001 μg/ml and incubated for 30 minutes. Cells were pelleted (no wash) and resuspended in 20 μl of LOB7/6 diluted in FACS buffer or B-B20 at diluted in FACS buffer and incubated for 30 minutes. Cells were washed twice with FACS buffer and resuspended in 20 μl of BV421 anti-mouse Ig diluted in FACS buffer and incubated for 30 minutes. Cells were washed twice with PBS and resuspended in 100 μl of 1% formaldehyde in PBS and incubated for 20 minutes. Cells were washed twice with FACS buffer and resuspended in 200 μl of FACS buffer for analysis.

FAGS staining controls included an unstained control, no blocking control and no primary antibody control. Cell staining was analysed using a FACS Canto II flow cytometer (BD). PMT voltages and gates for analysis were set using the FACS staining controls.

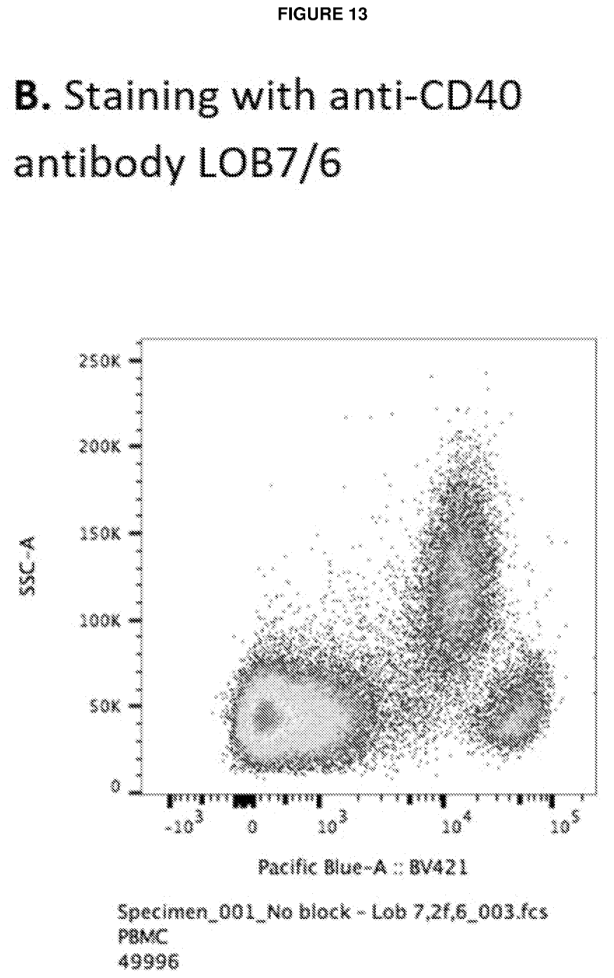

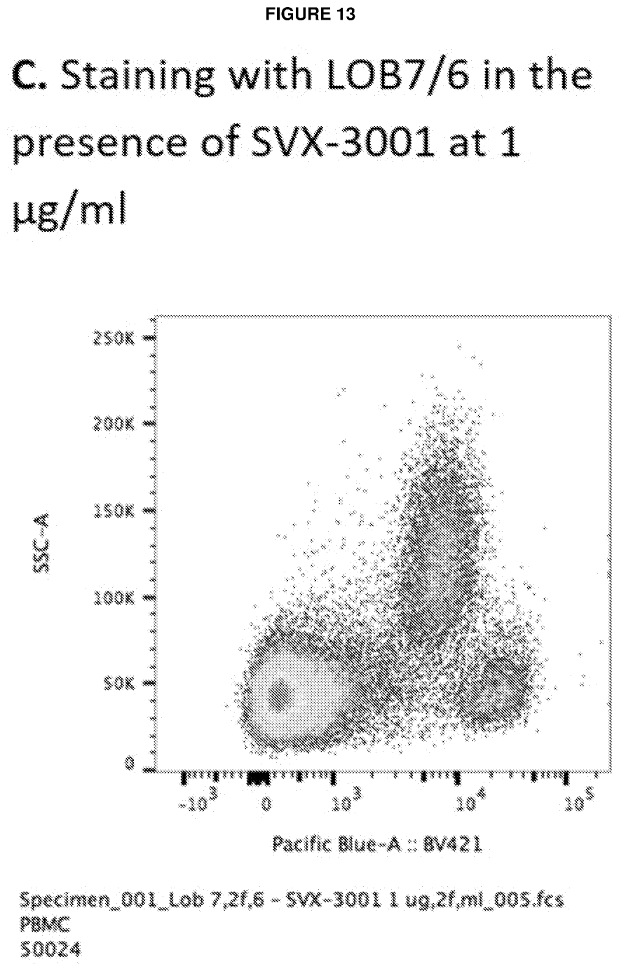

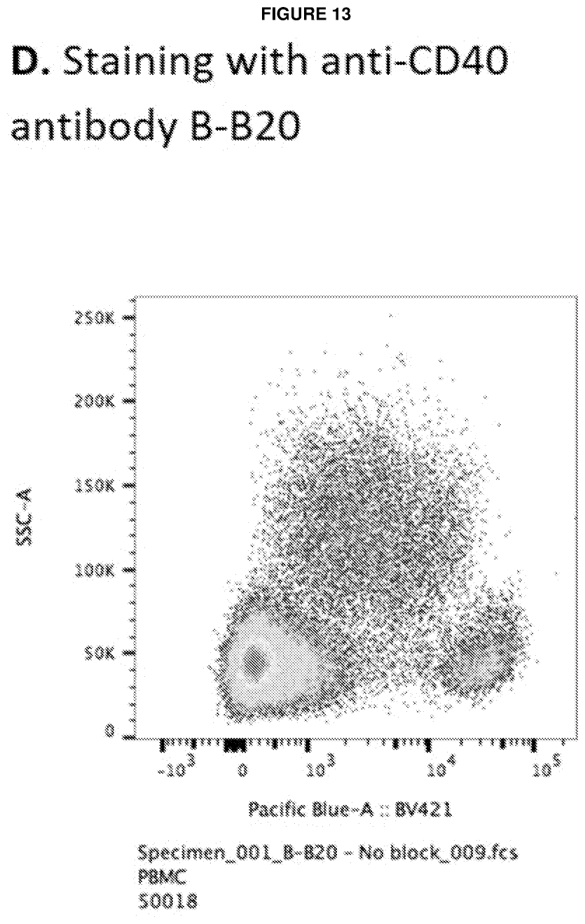

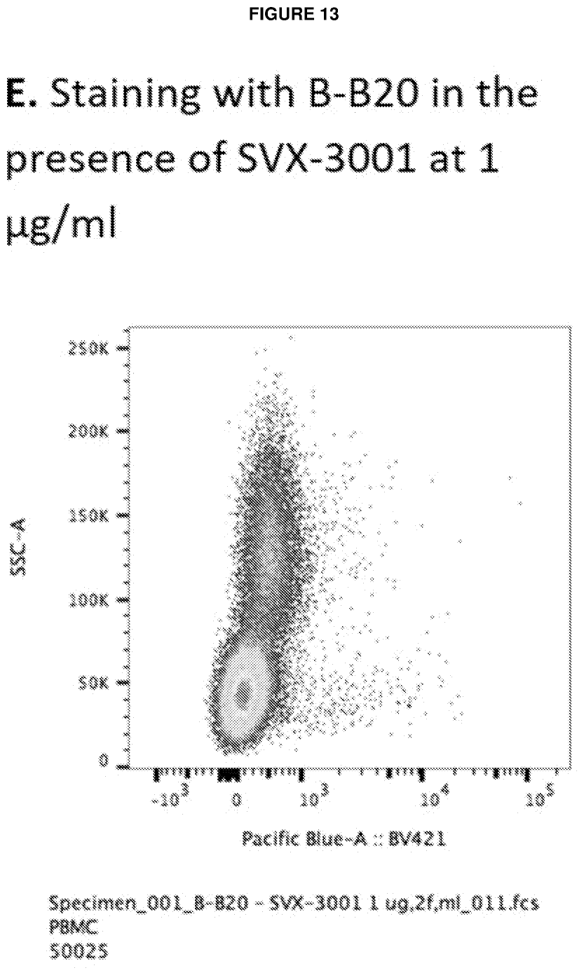

Results: Staining of human PBMC with anti-CD40 antibody B-B20 was blocked by SVX-3001 and staining with anti-CD40 antibody LOB7/6 was not blocked by SVX-3001 (FIG. 13). (A) No stain control. (B) Staining with LOB7/6. (C) Staining with LOB7/6 in the presence of SVX-3001 at 1 μg/ml. (D) Staining with B-B20. (E) Staining with B-B20 in the presence of SVX-3001 at 1 μg/ml).

Conclusions: Antibodies SVX-3001 and B-B20 bind to the same or an overlapping epitope on the human CD40 molecule. Antibodies SVX-3001 and LOB7/6 bind to a different epitope on the human CD40 molecule.

### Example 7—Antibody Competition Studies by Surface Plasmon Resonance

The ability of SVX-3001 to compete with a range of anti-CD40 antibodies was measured by surface plasmon resonance.

Method: Pairwise binding analysis by surface plasmon resonance (SPR) was carried out using the Biacore T200 (GE Healthcare). HBS-EP+ buffer was used as running buffer throughout.

Recombinant human CD40 extracellular domain with a C-terminal human IgG1 Fc domain and 6His tag (CD40-Fc; BioLegend) was immobilized to the surface of a Series S Sensor Chip CM5 (GE Healthcare) using an Amine Coupling Kit (GE Healthcare). CD40-Fc was diluted to 25 μg/ml in 10 mM Sodium acetate buffer pH 5.0 for immobilisation. Target immobilisation level: 1000 RU. Wash solution: Ethanolamine.

Reagents used for competition studies were purified SVX-3001 antibody, recombinant CD40L (BioLegend), and purified anti-CD40 antibodies Lob 7/4 IgG1 (University of Southampton), CP-870,893 IgG1 (University of Southampton), S2C6 (Mabtech), G28.5 (BioXCell) and LOB7/6 (LSBio).

Pairwise binding analysis was carried out by binding the first sample (sample 1) at a concentration sufficient to saturate the immobilised CD40-Fc on the surface following injection for 180 seconds at 10 μI/min. This concentration was in the range of 12.5 to 200 μg/ml for the reagents being tested. The second sample (sample 2) was then injected for 180 seconds at 10 μI/min. Regeneration was carried out using Glycine-HCl buffer pH 1.5, injection for 30 seconds at 10 μI/min with 5 seconds stabilisation before the next pair was tested. Pairs where the sample 1 and sample 2 were identical were used to confirm saturation and to set a baseline response.

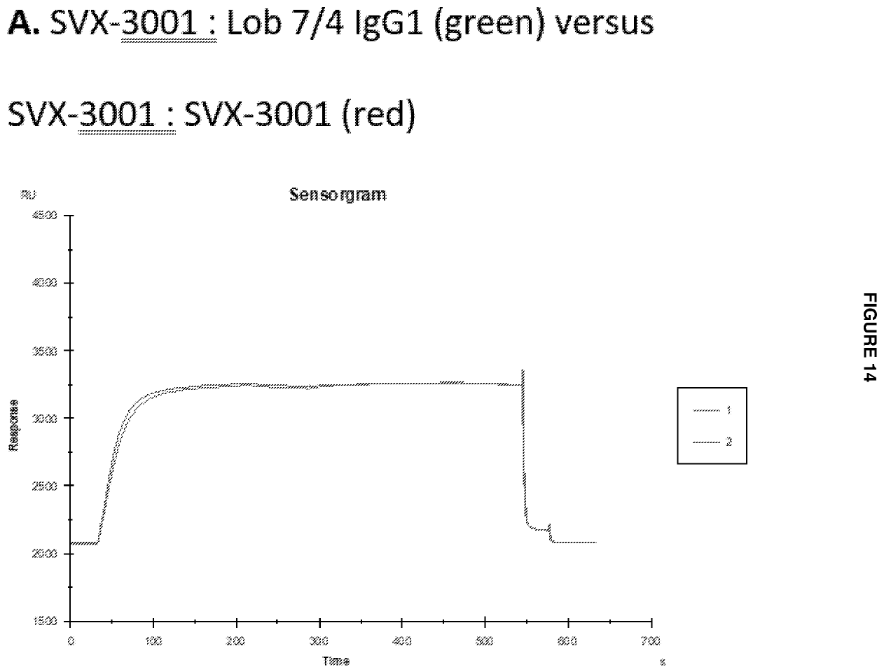

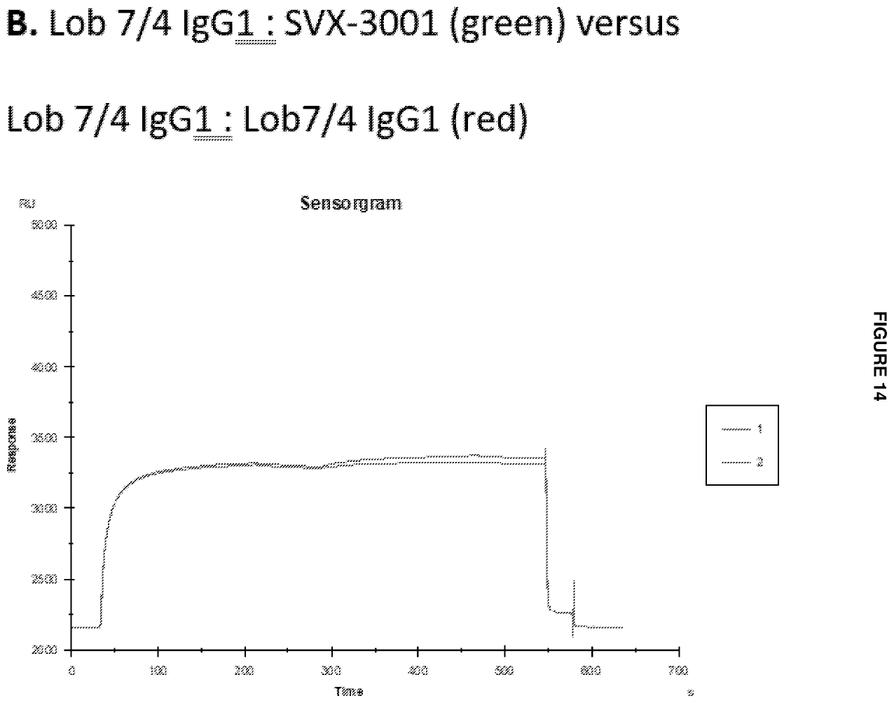

Results: Biacore T200 sensograms for epitope mapping by pairwise binding for SVX-3001, Lob 714 IgG1 and LOB7/6 are shown in FIG. 14. Results for all pairs that were tested are summarised in Table 3.

TABLE 3
Blocking of sample 2 by sample 1 with immobilised CD40-Fc

| | | | CP- | | | | |
|---|---|---|---|---|---|---|---|
| | SVX- | Lob 7/4 | 870,893 | | | LOB7/ | |
| Sample 1 | 3001 | IgG1 | IgG1 | S2C6 | G28.5 | 6 | CD40L |
| SVX-3001 | Yes | Yes | Yes | Yes | Yes | No | No |
| Lob7/4 | Yes | Yes | Yes | Yes | Yes | No | No |
| IgG1 | | | | | | | |
| CP- | Yes | — | — | — | — | — | — |
| 870,893 | | | | | | | |
| IgG1 | | | | | | | |
| S2C6 | Yes | — | — | — | — | — | — |
| G28.5 | Yes | — | — | — | — | — | — |
| LOB7/6 | No | — | — | — | — | — | — |
| CD40L | No | — | — | — | — | — | — |

Conclusions: SVX-3001 binds to an epitope on human CD40 that is the same as or overlaps with the epitopes for antibodies Lob 7/4 IgG1, CP-870,893 IgG1, S2C6 and G28.5. SVX-3001 binds to a different epitope that does not overlap with the epitope for antibody LOB7/6. The epitope for SVX-3001 does not overlap with the binding site for CD40L.

Antibodies Lob 7/4, SGN40 (which was derived from antibody S2C6) and CP-870,893 have all been reported to bind to epitopes in the CRD1 region of human CD40 (Cancer Cell 33, 664-675, Apr. 9, 2018). The inference from the SPR data is that SVX-3001 also binds to an epitope in the CRD1 region of human CD40 (P25942; Cys26-Cys59).

### Example 8—Antibody Affinity Studies by Surface Plasmon Resonance

The affinity of SVX-3001 to the extracellular domain of CD40 was measured using surface plasmon reference.

Method: Kinetic analysis by surface plasmon resonance (SPR) was carried out using the Biacore T200 (GE Healthcare). HBS-EP+ buffer was used as running buffer throughout.

Purified SVX-3001 was immobilised to the surface of a Series S Sensor Chip CM5 (GE Healthcare) using an Amine Coupling Kit (GE Healthcare). SVX-3001 was diluted to 25 μg/ml in 10 mM Sodium acetate buffer pH 5.0 for immobilisation. Target immobilisation level: 600 RU. Wash solution: Ethanolamine. The reference surface for analysis was either untreated or had human IgG1 isotype control (BioLegend) immobilised to the surface by the same process used for SVX-3001.

Reagents used for kinetics studies were recombinant human CD40 extracellular domain with C-terminal 6His tag (CD40-6His; Novoprotein) and recombinant human CD40 extracellular domain with a C-terminal human IgG1 Fc domain and 6His tag (CD40-Fc; BioLegend).

Injection parameters for the sample were contact time: 120 seconds, flow rate: 30 μI/min, dissociation time: 300 seconds. Injection parameters for regeneration were contact time: 30 seconds, flow rate: 30 μI/min, stabilisation period: 0 seconds. Regeneration solution was either 10 mM glycine-HCl buffer pH 1.5 or 50 mM NaOH. The concentrations of CD40-His and CD40-Fc used for the kinetic analysis were 2 mM, 4 mM, 8 mM (in duplicate), 16 mM and 32 mM. The model 1:1 binding was used for curve fitting.

Results: Kinetic and affinity constants for SVX-3001 from three experiments with CD40-Fc as analyte and three experiments with CD40-Fc as analyte are shown in Table 4.

TABLE 4
Kinetics and affinity constants for SVX-3001

| Analyte | Parameter | Value 1 | Value 2 | Value 3 | Average |
|---|---|---|---|---|---|
| CD40-6His | kₐ (M⁻¹s⁻¹) | 7.150 × | 6.283 × | 5.759 × | 6.397 × |
| | | 10⁵ | 10⁵ | 10⁵ | 10⁵ |
| | k_{d} (s⁻¹) | 3.808 × | 4.085 × | 3.748 × | 3.880 × |
| | | 10⁻³ | 10⁻³ | 10⁻³ | 10⁻³ |
| | K_{D} (nM) | 5.325 | 6.501 | 6.508 | 6.111 |
| CD40-Fc | kₐ (M⁻¹s⁻¹) | 7.174 × | 4.970 × | 6.610 × | 6.21 × |
| | | 10⁵ | 10⁵ | 10⁵ | 10⁵ |
| | k_{d} (s⁻¹) | 2.318 × | 5.550 × | 2.033 × | 3.300 × |
| | | 10⁻⁴ | 10⁻⁴ | 10⁻⁴ | 10⁻⁴ |
| | K_{D} (nM) | 0.3232 | 1.117 | 0.3076 | 0.5826 |

Conclusions: The calculated equilibrium dissociation constant (K_{D}) for SVX-3001 using CD40-6His as analyte was 6.111×10⁻⁹ M (6.111 nM) and the calculated K_{D} for SVX-3001 using CD40-Fc as analyte was 5.826×10⁻¹⁰ M (0.5826 nM). The calculated association rate constants (kₐ) using both analytes were similar, so the difference in K_{D} appears to be due to the difference in dissociation rate constants (k_{d}) for the two analytes.

### Example 9—Activation of Monocyte-Derived Dendritic Cells with Anti-CD40 Antibody

Stimulation of monocyte-derived dendritic cells (moDC) in the presence of SVX-3001, CD40L or LPS with IFN-γ was measured by reference to the expression of co-stimulatory molecules.

Method: Human buffy coat samples were obtained from the Australian Red Cross Blood Service (Ethics approval: RDHS-243-15, Human Research Ethics Office, Curtin University). Peripheral blood mononuclear cells (PBMC) were isolated from human buffy coat samples by density centrifugation using Ficoll-Paque PLUS (GE Healthcare Life Sciences). Human PBMC were seeded into 6-well plates (Corning-Falcon) at a density of 5×10⁶ cells/well in RPMI (Gibco)+10% FCS (Hyclone) and incubated for 2 hours at 37° C. to allow monocytes to adhere to the plastic. Medium with non-adherent cells was then removed.

Adherent monocytes were differentiated into monocyte-derived dendritic cells (moDC) by culturing for 7 days in RPMI+10% FCS with 80 ng/ml recombinant human GM-CSF (Shenandoah Biotechnology), 10 ng/ml recombinant human IL-4 (Shenandoah Biotechnology) and 10 μg/ml polymyxin B (Sigma-Aldrich). Medium containing GM-CSF, IL-4 and polymyxin B was replaced on day 4.

Cells were stimulated on day 7 in medium with GM-CSF and IL-4 without polymyxin B. Stimulation conditions included medium control (no stimulation), 1 μg/ml SVX-3001, 1 μg/ml human IgG1 isotype control (BioLegend), 0.67 mg/ml recombinant CD40L (BioLegend) and 1 μg/ml LPS (Sigma-Aldrich) with 20 ng/ml recombinant human IFN-γ (Shenandoah Biotechnology). On day 9 cells were treated with 1×Brefeldin A Solution (BioLegend) for 4 hours before cells were harvested and stained for FACS analysis (48-hours stimulation).

FACS staining was carried out in 96-well U-bottom plates (Corning-Falcon). The FACS buffer used for wash steps and to dilute antibodies consisted of PBS with 1% BSA (Sigma), 1% FCS (Hyclone) and 0.01% w/v sodium azide (Sigma). Staining reagents included BUV805 anti-CD3 (BD), Alexafluor700 anti-CD14 (BioLegend), BV605 anti-CD11 b (BioLegend), BV711 anti-CD11c (BioLegend), APC anti-CD1a (BioLegend), PerCP-Cy5.5 anti-HLA-A,B,C (BioLegend), APC-H7 anti-HLA-DR (BD), FITC anti-CD80 (BioLegend), PE-Cy7 anti-CD83 (BioLegend), BUV395 anti-CD86 (BD), BV510 anti-PD-L1 (BioLegend), BV421 anti-IL-12 (BD) and Zombie UV (BioLegend).

Each staining sample contained moDC from 1 well of a 6-well plate used for cell culture and stimulation. Cells were washed twice with phosphate buffered saline (PBS) and resuspended in 100 μl of Zombie UV diluted in PBS and incubated for 15 minutes. Cells were washed twice with FACS buffer and resuspended in 100 μl of staining mix containing 50 μl Brilliant Stain Buffer (BD) and 50 μl of antibodies against cell surface markers diluted in FACS buffer and incubated for 30 minutes. Cells were washed twice with PBS and resuspended in 100 μl of Fixation/Permeabilization solution (BD) and incubated for 20 minutes. Cells were washed twice with Perm/Wash buffer (BD) and resuspended in 100 μl of staining mix containing 50 μl Brilliant Stain buffer and 50 μl of antibodies against intracellular markers diluted in Perm/Wash buffer. Cells were washed twice with Perm/Wash buffer and resuspended in 200 μl FACS buffer for analysis.

FACS staining controls included an unstained control and single stain controls. Cell staining was analysed using a LSRFortessa Cell Analyser (BD). PMT voltages and compensation values were set using the FACS staining controls. Gates for the analysis of moDC were size (FSC-A vs. SSC-A), singlets (FSC-A vs. FSC-H), live (Zombie UV negative), CD3⁻ CD14⁻ cells (CD3 vs. CD14) and CD11b+CD11c+ cells (CD11b vs. CD11c). The median fluorescence intensity (MFI) of cell surface markers was determined for the whole moDC population.

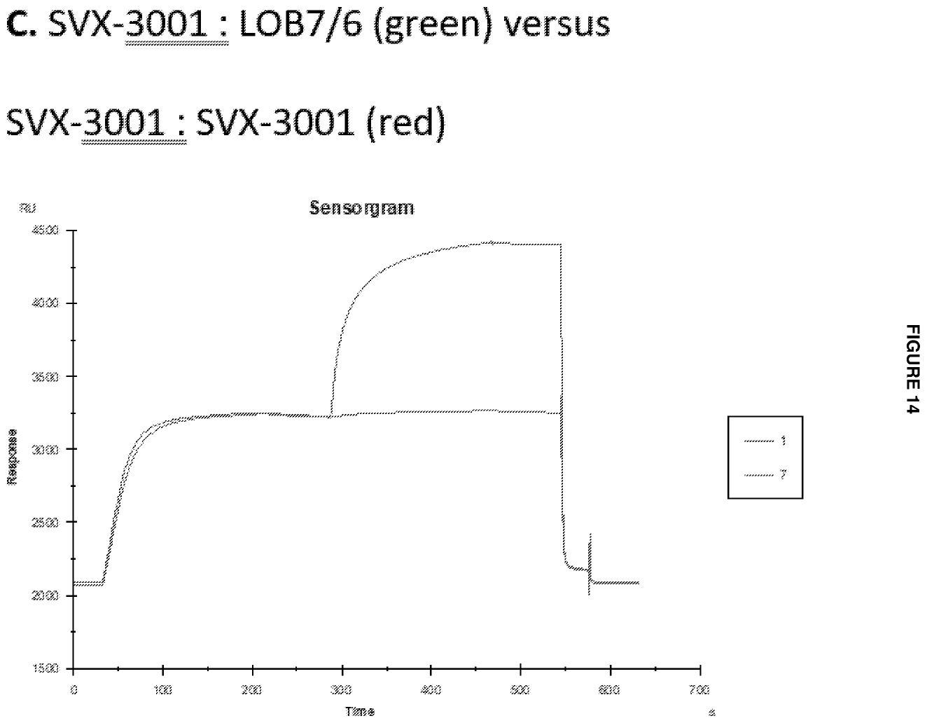

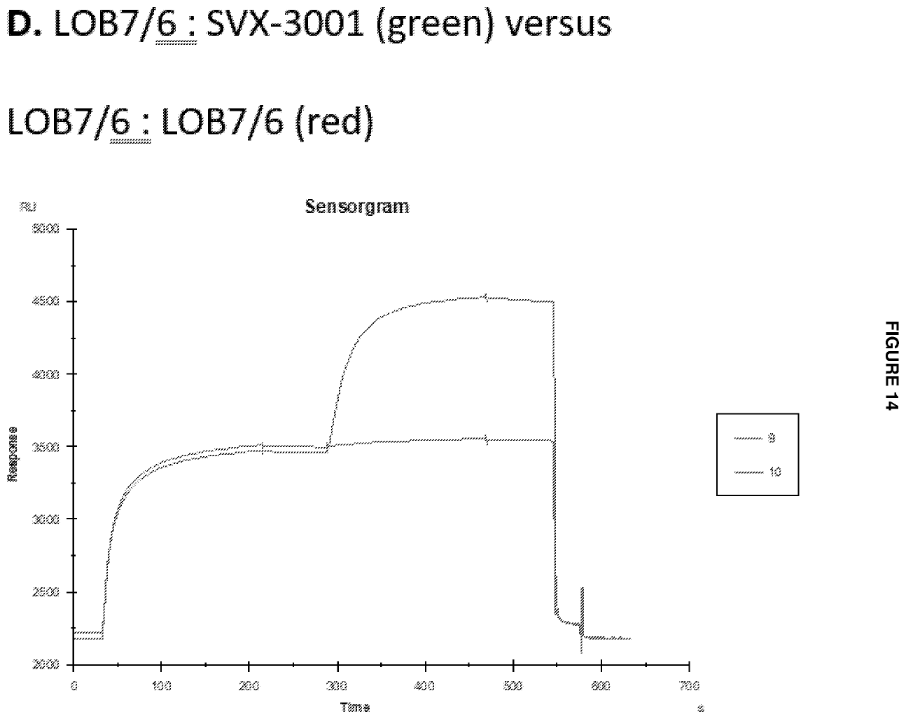

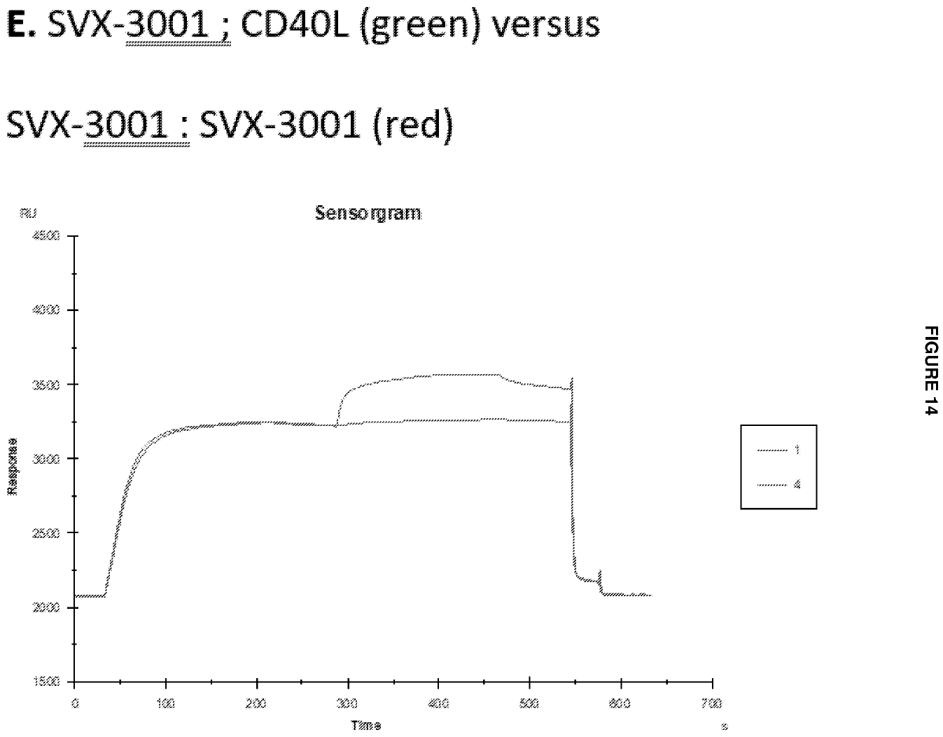

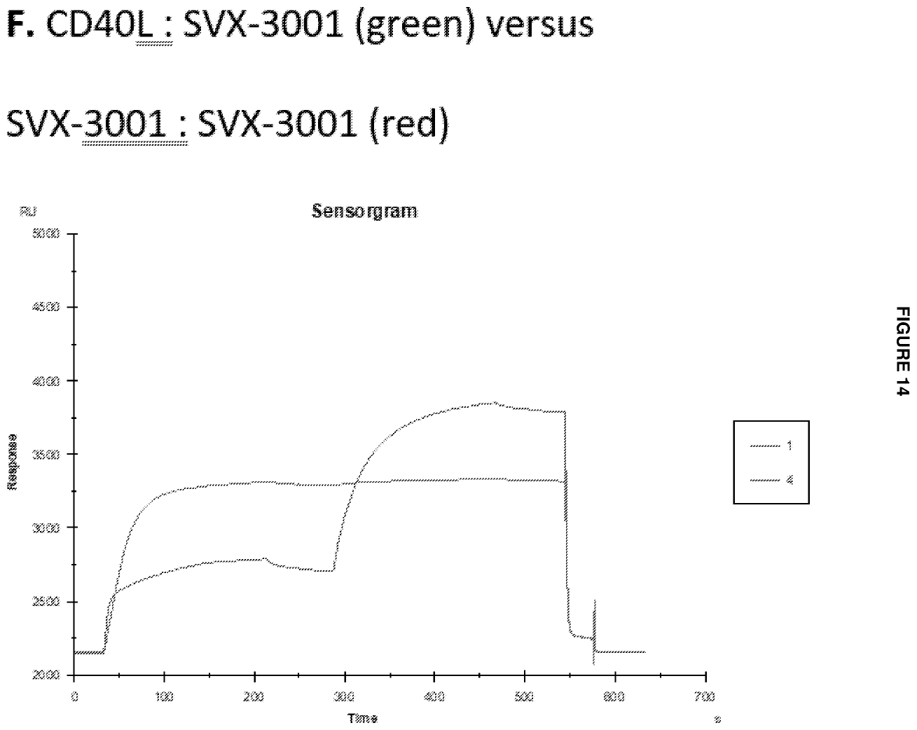

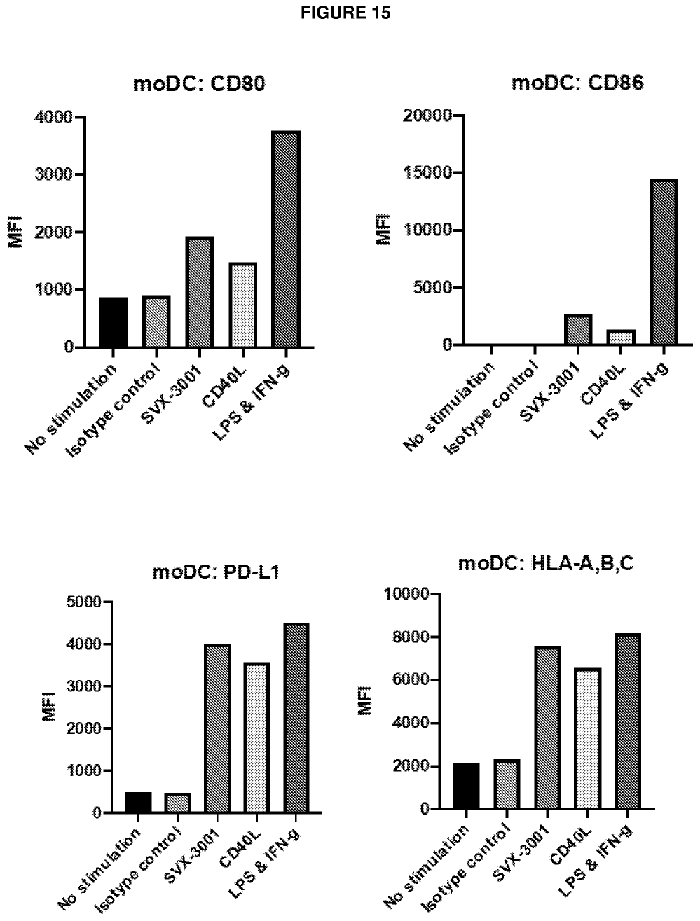

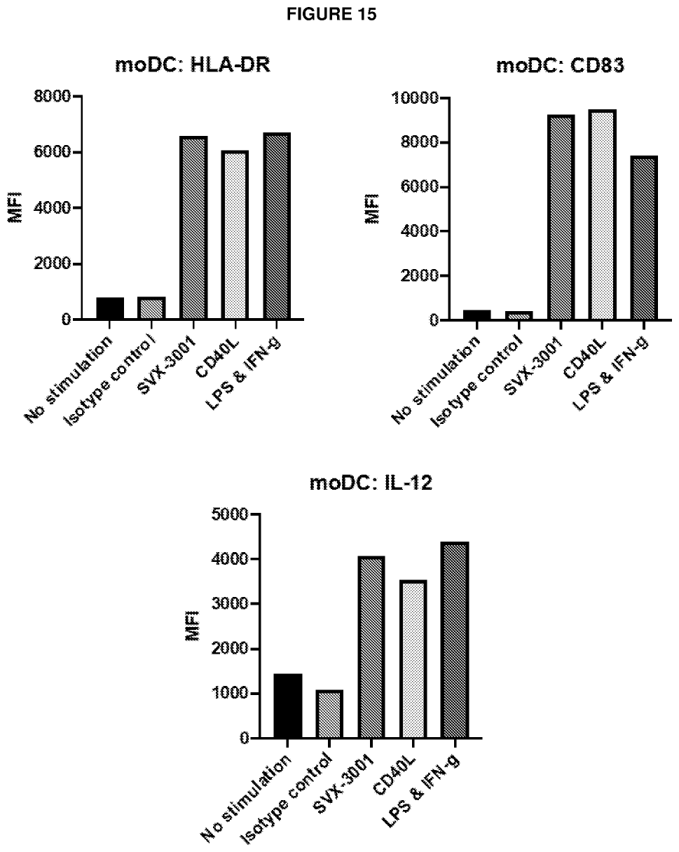

Results: Stimulation of moDC for 48 hours with SVX-3001, CD40L or LPS with IFN-γ resulted in up-regulation of cell surface markers HLA-A,B,C, HLA-DR, CD80, CD83 CD86 and PD-L1 and increased expression of IL-12, as shown in FIG. 15. These markers were not upregulated in unstimulated moDC or in moDC stimulated with a human IgG1 isotype control antibody.

Conclusions: Anti-CD40 antibody SVX-3001 has agonist activity on human monocyte-derived dendritic cells (moDC), which are an example of antigen presenting cells (APC), that results in increased expression of co-stimulatory molecules B7-1 (CD80), B7-2 (CD86) and PD-L1 (CD274) along with HLA-A,B,C (MHC class I), HLA-DR (MHC class II), CD83 and the pro-inflammatory cytokine IL-12. This agonist activity is dependent on the antigen-binding domain of the antibody, as this response is not seen to a human IgG1 isotype control antibody that shares the same Fc domain.

### Example 10—Activation of Monocyte-Derived Dendritic Cells with Anti-CD40 Antibody—Dose Response

Stimulation of monocyte-derived dendritic cells (moDC) in the presence of SVX-3001 was measured by reference to the expression of co-stimulatory molecules. The dose response of the stimulatory effect of SVX-3001 was also measured.

Method: Human buffy coat samples were obtained from the Australian Red Cross Blood Service (Ethics approval: RDHS-243-15, Human Research Ethics Office, Curtin University). Peripheral blood mononuclear cells (PBMC) were isolated from human buffy coat samples by density centrifugation using Ficoll-Paque PLUS (GE Healthcare Life Sciences). Human PBMC were seeded into 6-well plates (Corning-Falcon) at a density of 5×10⁶ cells/well in RPMI (Gibco)+10% FCS (Hyclone) and incubated for 2 hours at 37° C. to allow monocytes to adhere to the plastic. Medium with non-adherent cells was then removed.

Adherent monocytes were differentiated into monocyte-derived dendritic cells (moDC) by culturing for 7 days in RPMI+10% FCS with 80 ng/ml recombinant human GM-CSF (Shenandoah Biotechnology), 10 ng/ml recombinant human IL-4 (Shenandoah Biotechnology) and 10 μg/ml polymyxin B (Sigma-Aldrich). Medium containing GM-CSF, IL-4 and polymyxin B was replaced on day 4.

Cells were stimulated on day 7 in medium with GM-CSF and IL-4 without polymyxin B. Stimulation conditions included anti-CD40 antibody SVX-3001 at 1 μg/ml, 0.316 μg/ml, 0.1 μg/ml, 0.0316 μg/ml and 0.01 μg/ml. Controls included medium alone (unstimulated), 1 μg/ml human IgG1 isotype control (BioLegend) and 1 μg/ml LPS (Sigma-Aldrich) with 20 ng/ml recombinant human IFN-γ (Shenandoah Biotechnology). On day 9 cells were treated with 1×Brefeldin A Solution (BioLegend) for 4 hours before cells were harvested and stained for FACS analysis (48-hours stimulation).

FACS staining was carried out in 96-well U-bottom plates (Corning-Falcon). The FACS buffer used for wash steps and to dilute antibodies consisted of PBS with 1% BSA (Sigma), 1% FCS (Hyclone) and 0.01% w/v sodium azide (Sigma). Staining reagents included BUV805 anti-CD3 (BD), Alexafluor700 anti-CD14 (BioLegend), BV605 anti-CD11 b (BioLegend), BV711 anti-CD11c (BioLegend), APC anti-CD1a (BioLegend), PerCP-Cy5.5 anti-HLA-A,B,C (BioLegend), APC-H7 anti-HLA-DR (BD), FITC anti-CD80 (BioLegend), PE-Cy7 anti-CD83 (BioLegend), BUV395 anti-CD86 (BD), BV510 anti-PD-L1 (BioLegend), BV421 anti-IL-12 (BD) and Zombie UV (BioLegend).

Each staining sample contained moDC from 1 well of a 6-well plate used for cell culture and stimulation. Cells were washed twice with phosphate buffered saline (PBS) and resuspended in 100 μl of Zombie UV diluted in PBS and incubated for 15 minutes. Cells were washed twice with FACS buffer and resuspended in 100 μl of staining mix containing 50 μl Brilliant Stain Buffer (BD) and 50 μl of antibodies against cell surface markers diluted in FACS buffer and incubated for 30 minutes. Cells were washed twice with PBS and resuspended in 100 μl of Fixation/Permeabilization solution (BD) and incubated for 20 minutes. Cells were washed twice with Perm/Wash buffer (BD) and resuspended in 100 μl of staining mix containing 50 μl Brilliant Stain buffer and 50 μl of antibodies against intracellular markers diluted in Perm/Wash buffer. Cells were washed twice with Perm/Wash buffer and resuspended in 200 μl FACS buffer for analysis.

FACS staining controls included an unstained control and single stain controls. Cell staining was analysed using a LSRFortessa Cell Analyser (BD). PMT voltages and compensation values were set using the FACS staining controls. Gates for the analysis of moDC were size (FSC-A vs. SSC-A), singlets (FSC-A vs. FSC-H), live (Zombie UV vs SSC-A), CD3⁻ CD14⁻ cells (CD3 vs. CD14), CD11b+CD11c+ cells (CD11 b vs. CD11c) and CD1a+ cells (CD1a vs. SSC-A). The median fluorescence intensity (MFI) of cell surface markers was determined for the whole moDC population.

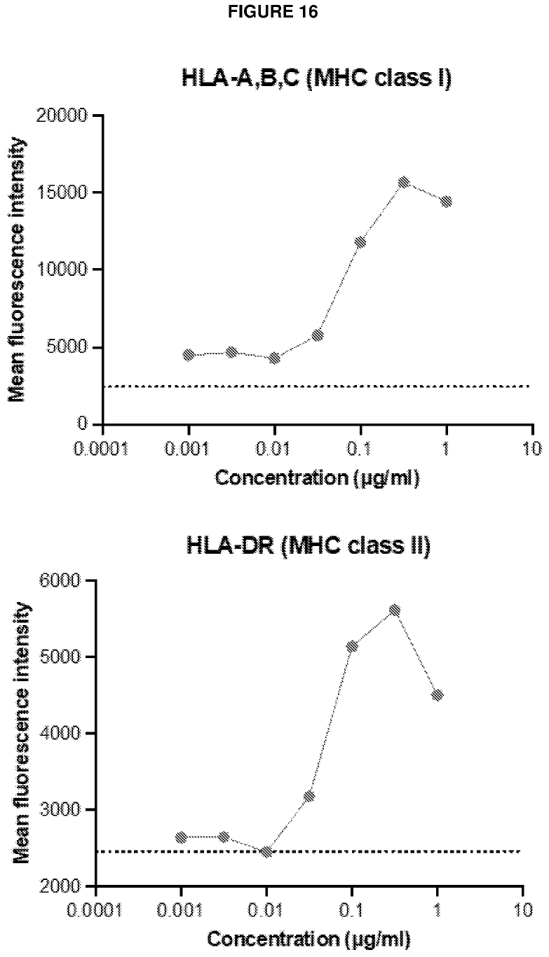

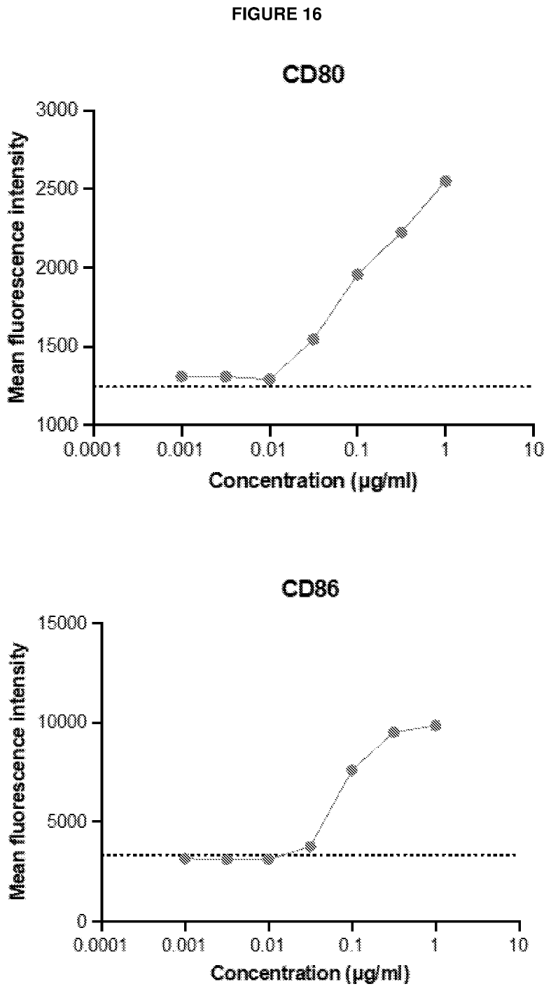

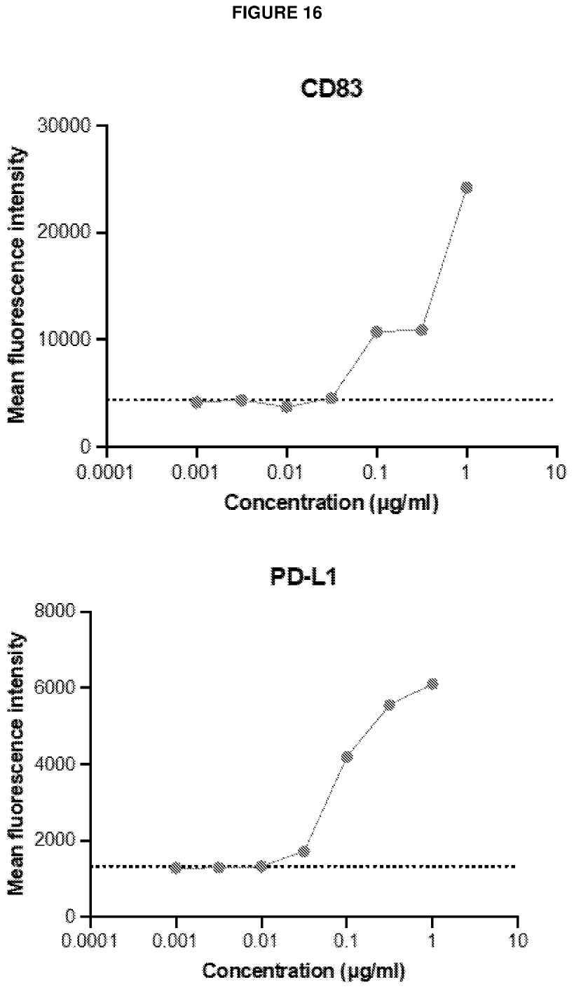

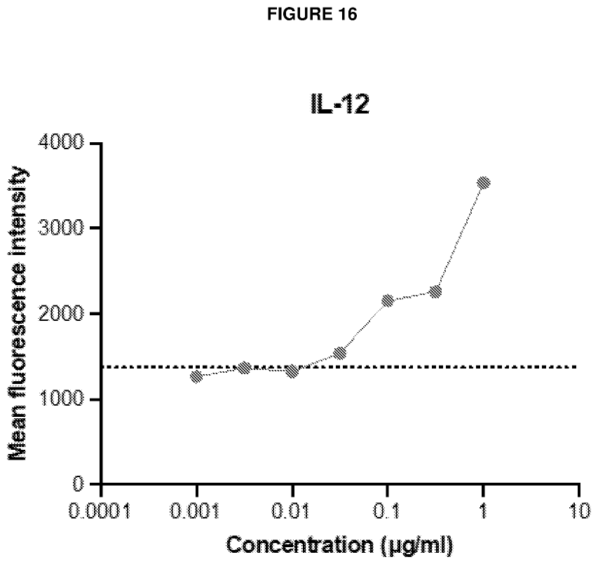

Results: Stimulation of human moDC for 48 hours with anti-CD40 antibodies SVX-3001, APX005M and CP-870,893 resulted in up-regulation of cell surface markers HLA-A,B,C (MHC class I), HLA-DR (MHC class II), CD80, CD83, CD86 and PD-L1 and the cytokine IL-12 in a dose-dependent manner. FIG. 16 shows the mean fluorescent intensity (MFI) for FACS staining of moDC stimulated with SVX-3001 for 48 hours at different concentrations. The MFI of unstimulated moDC is shown as a dotted line.

Conclusions: Anti-CD40 antibody SVX-3001 provides a dose-dependent agonist signal to human moDC that results in the up-regulation of cell surface cell surface markers HLA-A,B,C, HLA-DR, CD80, CD83, CD86 and PD-L1 and the cytokine IL-12.

### Example 11—Gene Synthesis of Codon Optimized Sequences for SVX-3001

DNA sequences encoding the heavy chain of SVX-3001 and the light chain of SVX-3001 were optimized for expression in Homo sapiens using GeneOptimizer™ by GeneArt, Thermo Fisher Scientific, resulting in gene sequences 20ACGJQC_Selvax01HC (SEQ ID NO 21, FIG. 17) and 20ACGJRC_Selvax01LC (SEQ ID NO 22, FIG. 18). In FIGS. 17 and 18, the Kozak sequence is shown in bold and coding sequence for the heavy and light chains is underlined.

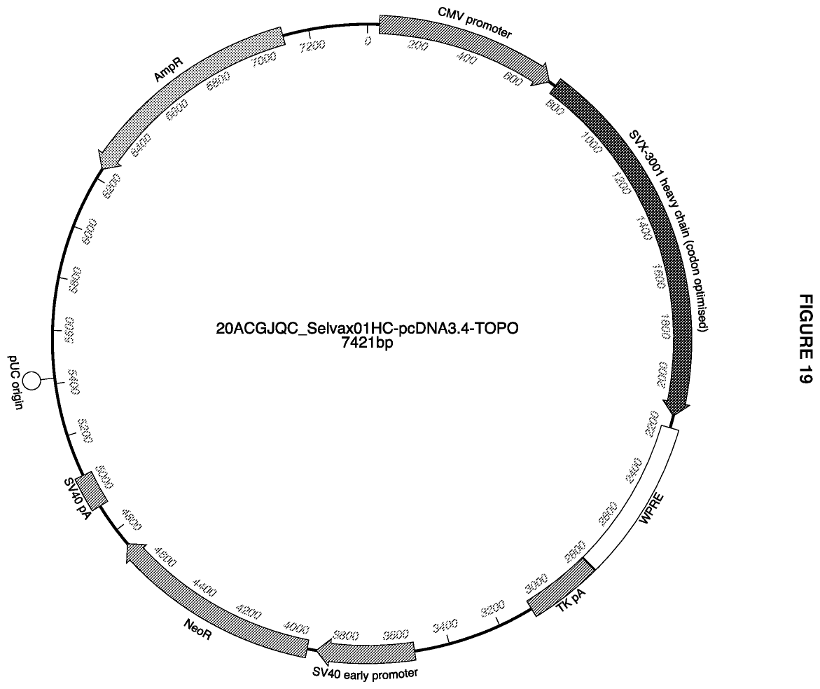

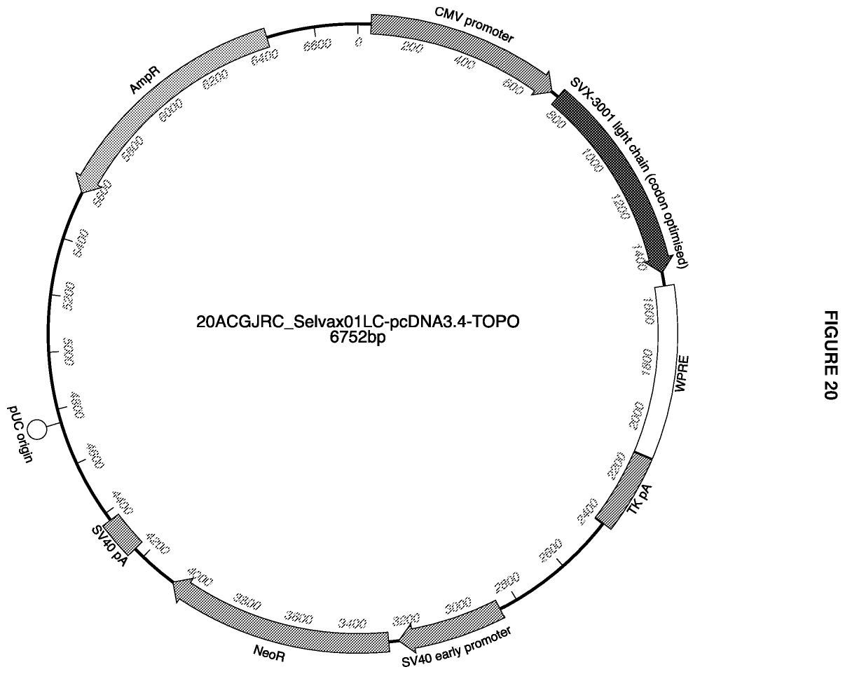

Gene sequences 20ACGJQC_Selvax01HC and 20ACGJRC_Selvax01LC were synthesised by GeneArt, Thermo Fisher Scientific and inserted into pcDNA3.4-TOPO to create plasmids 20ACGJQC_Selvax01HC-pcDNA3.4-TOPO (FIG. 19) and 20ACGJRC_Selvax01LC-pcDNA3.4-TOPO (FIG. 20).

1. An isolated agonist anti-CD40 antibody or fragment thereof which comprises (i) a V_{H} chain comprising three CDRs and (ii) a V_{L} chain comprising three CDRs, wherein one or more heavy chain complementary determining regions (CDRHs) is selected from the group consisting of:
(d) a CDRH1 sequence comprising SEQ ID NO:1;
(e) a CDRH2 sequence comprising SEQ ID NO:2; or
(f) a CDRH2 sequence comprising SEQ ID NO:2 that contains one or two amino acid substitutions, deletions or insertions.

2. The isolated agonist anti-CD40 antibody according to claim 1 additionally comprising:
(a) a CDRH3 sequence comprising SEQ ID NO:3;
(b) a CDRL1 sequence comprising SEQ ID NO:4;
(c) a CDRL2 sequence comprising SEQ ID NO:5; or
(d) a CDRL3 sequence comprising SEQ ID NO:6.

3. The isolated agonist anti-CD40 antibody according to claim 1 or 2 comprising:
A:
(a) a CDRH1 sequence comprising SEQ ID NO:1; or
(b) a CDRH2 sequence comprising SEQ ID NO:2; and
B:
(c) a CDRH3 sequence comprising SEQ ID NO:3;
(d) a CDRL1 sequence comprising SEQ ID NO:4;
(e) a CDRL2 sequence comprising SEQ ID NO:5; or
(f) a CDRL3 sequence comprising SEQ ID NO:6.

4. The isolated agonist anti-CD40 antibody according to claims 1 to 3 comprising:
(a) a CDRH1 sequence comprising SEQ ID NO:1;
(b) a CDRH2 sequence comprising SEQ ID NO:2;
(c) a CDRH3 sequence comprising SEQ ID NO:3;
(d) a CDRL1 sequence comprising SEQ ID NO:4;
(e) a CDRL2 sequence comprising SEQ ID NO:5; and
(f) a CDRL3 sequence comprising SEQ ID NO:6.

5. The isolated agonist anti-CD40 antibody according to any of the preceding claims comprising a heavy chain variable region of SEQ ID NO: 7.

6. The isolated agonist anti-CD40 antibody according to any of the preceding claims comprising a light chain variable region of SEQ ID NO:8.

7. An isolated agonist anti-CD40 antibody according to any of the preceding claims wherein the antibody comprises the heavy chain of SEQ ID NO: 19 or the light chain of SEQ ID NO:20.

8. The isolated agonist anti-CD40 antibody according to any of the preceding claims comprising a heavy chain variable region of SEQ ID NO: 7 and a light chain variable region of SEQ ID NO:8.

9. An isolated agonist anti-CD40 antibody according to anyone of the preceding claims wherein the isolated agonist anti-CD40 antibody is a humanized antibody directed against human CD40.

10. An isolated agonist anti-CD40 antibody according to claim 8 wherein the isolated agonist anti-CD40 antibody is a monoclonal antibody.

11. An isolated agonist anti-CD40 antibody according to any of the preceding claims wherein the antibody is an IgG antibody.

12. An isolated agonist anti-CD40 antibody according to any of the preceding claims wherein the antibody comprises the heavy chain of SEQ ID NO: 19 and the light chain of SEQ ID NO:20.

13. A pharmaceutical composition comprising at least one agonist anti-CD40 antibody according to any one of claims 1 to 12.

14. A pharmaceutical composition comprising at least one isolated agonist anti-CD40 antibody according to any one of claims 1 to 12 and a pharmaceutically acceptable excipient.

15. A pharmaceutical composition according to claim 13 or 14 wherein the composition further comprises an additional active agent.

16. A pharmaceutical composition according to claim 15 wherein the additional active agent is selected from the group consisting of a radioisotope, radionuclide, a toxin, or a therapeutic and a chemotherapeutic group.

17. A method of making the agonist anti-CD40 antibody according to any one of claims 1 to 12, comprising the step of preparing said agonist anti-CD40 antibody from a host cell that secretes said agonist anti-CD40 antibody.

18. A method for treating or preventing a condition associated with malignancy in a patient, comprising administering to a patient in need thereof an effective amount of at least one isolated agonist anti-CD40 antibody according to any one of claims 1 to 12.

19. A method for increasing antigen presentation by APCs (including macrophages, DCs, and B cells), in a subject comprising administering an effective amount of at least one isolated agonist anti-CD40 antibody according to any one of claims 1 to 12.

20. A method of activating antigen presenting cells in a subject comprising administering an effective amount of at least one isolated agonist anti-CD40 antibody according to any one of claims 1 to 12.

21. A method of enhancing the expression of MHC and/or immune costimulatory molecules, in a subject comprising administering an effective amount of at least one isolated agonist anti-CD40 antibody according to any one of claims 1 to 12.

22. A method of stimulating the production of pro-inflammatory cytokines, in a subject comprising administering an effective amount of at least agonist anti-CD40 antibody according to any one of claims 1 to 12.

23. A method of inducing T cell activation, in a subject comprising administering an effective amount of at least one agonist anti-CD40 antibody according to any one of claims 1 to 12.

24. A method to mimic the signal of CD40L and substitute for the function of CD4+ lymphocytes, in a subject comprising administering an effective amount of at least one agonist anti-CD40 antibody according to any one of claims 1 to 12.

25. A method for overcoming T cell tolerance in tumor-bearing subjects or evoking effective cytotoxic T cell responses or enhance the efficacy of anti-tumor vaccines, in a subject comprising administering an effective amount of at least one agonist anti-CD40 antibody according to any one of claims 1 to 12.

26. A method of promoting the secretion of autoantibodies directed against antigen expressed on tumour cells by B cells, in a subject comprising administering an effective amount of an agonist anti-CD40 antibody according to any one of claims 1 to 12.

27. A method of upregulating costimulatory markers and releasing IL-12 to activate CD8+ T cells and stimulating a specific cytotoxic T-cell response to cross-presented tumour antigens in a subject comprising administering an effective amount of an agonist anti-CD40 antibody according to any one of claims 1 to 12.

28. A pharmaceutical composition comprising an effective amount of at least one agonist anti-CD40 antibody according to any one of claims 1 to 12 together with at least a second immune enhancing agent.

29. A pharmaceutical composition according to claim 28 wherein the immune enhancing agent is selected from IL-2, TLR-7 agonists, or systemic cytotoxic chemotherapeutic agents.

30. A method for treating or preventing a condition associated with malignancy in a patient, comprising administering to a patient in need thereof an effective amount of at least one isolated agonist anti-CD40 antibody according to any one of claims 1 to 12 and an effective amount of at least a second immune enhancing agent.

31. A pharmaceutical composition comprising at least one agonist anti-CD40 antibody according to any one of claims 1 to 12 and IL-2.

32. A method for treating or preventing a condition associated with malignancy in a patient, comprising administering to a patient in need thereof an effective amount of each of, at least one agonist anti-CD40 antibody according to any one of claims 1 to 12 and IL-2.

33. A method for increasing antigen presentation by APCs (including macrophages, DCs, and B cells), in a subject comprising administering an effective amount of an agonist anti-CD40 antibody according to any one of claims 1 to 12 and IL-2.

34. A method of activating antigen presenting cells in a subject comprising administering an effective amount of at least one isolated agonist anti-CD40 antibody according to any one of claims 1 to 12 and IL-2.

35. A method of promoting the secretion of autoantibodies directed against antigen expressed on tumour cells by B cells, in a subject comprising administering an effective amount of an agonist anti-CD40 antibody according to any one of claims 1 to 12 and IL-2.

36. A method of upregulating costimulatory markers and releasing IL-12 to activate CD8+ T cells and stimulating a specific cytotoxic T-cell response to cross-presented tumour antigens in a subject comprising administering an effective amount of an agonist anti-CD40 antibody according to any one of claims 1 to 12 and IL-2.

37. A method for increasing antigen presentation by APCs (including macrophages, DCs, and B cells), in a subject comprising administering an effective amount of each of, at least one agonist anti-CD40 antibody according to any one of claims 1 to 12 and IL-2.

38. A method of enhancing the expression of MHC and/or immune costimulatory molecules, in a subject comprising administering an effective amount of each of, at least one agonist anti-CD40 antibody according to any one of claims 1 to 12 and IL-2.

39. A method of stimulating the production of pro-inflammatory cytokines (such as IL-12), in a subject comprising administering an effective amount of each of, at least one agonist anti-CD40 antibody according to any one of claims 1 to 12 and IL-2.

40. A method of inducing T cell activation, in a subject comprising administering an effective amount of each of, at least one agonist anti-CD40 antibody according to any one of claims 1 to 12 and IL-2.

41. A method to mimic the signal of CD40L and substitute for the function of CD4+ lymphocytes, in a subject comprising administering an effective amount of each of, at least one agonist anti-CD40 antibody according to any one of claims 1 to 12 and IL-2.

42. A method for overcoming T cell tolerance in tumor-bearing subjects or evoking effective cytotoxic T cell responses or enhance the efficacy of anti-tumor vaccines, in a subject comprising administering an effective amount of each of, at least one agonist anti-CD40 antibody according to any one of claims 1 to 12 and IL-2.

43. A nucleic acid molecule encoding an agonist anti-CD40 antibody according to anyone of claims 1 to 12.

44. A nucleic acid molecule encoding the heavy chain of an agonist anti-CD40 antibody comprising SEQ ID NO 9.

45. A nucleic acid molecule according to claim 43 where the molecule has at least 80, 80-85, 85-90, 90-95, 95-97, 97-99 or greater identity to SEQ ID NO 9.

46. A nucleic acid molecule according to any of claims 43 to 45 additionally comprising the light chain of an agonist anti-CD40 antibody comprising SEQ ID NO 10.

47. A nucleic acid molecule encoding the heavy chain of an agonist anti-CD40 antibody comprising SEQ ID NO 21.

48. A nucleic acid molecule according to claim 43 where the molecule has at least 80, 80-85, 85-90, 90-95, 95-97, 97-99 or greater identity to SEQ ID NO 21.

49. A nucleic acid molecule according to any of claims 46 to 48 additionally comprising the light chain of an agonist anti-CD40 antibody comprising SEQ ID NO 22.
